(19) United States
(12) Patent Application Publication
(10) Pub. No.: US 2011/0201628 A1
(43) Pub. Date: Aug. 18, 2011

(54) HETEROCYCLIC JAK KINASE INHIBITORS

(75) Inventors: Claudio Edmundo Chuaqui, Waltham, MA (US); Shan Huang, Waltham, MA (US); Stephanos Ioannidis, Waltham, MA (US); Jie Shi, Waltham, MA (US); Mei Su, Waltham, MA (US); Qibin Su, Waltham, MA (US)

(73) Assignee: AstraZeneca AB, Sodertalje (SE)

(21) Appl. No.: 13/119,028

(22) PCT Filed: Sep. 29, 2009
(86) PCT No.: PCT/GB2009/051273

§ 371 Date: Apr. 26, 2011

Related U.S. Application Data
(60) Provisional application No. 61/101,307, filed on Sep. 30, 2008.

(51) Int. Cl. A61K 31/519; C07D 491/04; C07D 487/04; C07D 471/04; C07D 239/70; C07D 239/72; A61K 31/517; A61P 35/00

(52) U.S. Cl. 514/260.1; 544/278; 544/280; 544/255; 544/253; 514/258.1; 514/262.1; 514/264.1; 514/265.1; 514/266.23

(57) ABSTRACT

The present invention relates to compounds of Formula (I) and to their salts, pharmaceutical compositions, methods of use, and methods for their preparation. These compounds provide a treatment for myeloproliferative disorders and cancer.

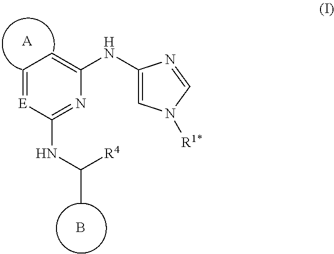

### FIELD OF THE INVENTION

The present invention relates to novel compounds, their pharmaceutical compositions and methods of use. In addition, the present invention relates to therapeutic methods for the treatment and prevention of cancers and to the use of these compounds in the manufacture of medicaments for the treatment and prevention of myeloproliferative disorders and cancers.

### BACKGROUND OF THE INVENTION

The JAK (Janus-associated kinase)/STAT (signal transducers and activators of transcription) signaling pathway is involved in a variety of hyperproliferative and cancer related processes including cell-cycle progression, apoptosis, angiogenesis, invasion, metastasis and evasion of the immune system (Haura et al., Nature Clinical Practice Oncology, 2005, 2(6), 315-324; Verna et al., Cancer and Metastasis Reviews, 2003, 22, 423-434).

The JAK family consists of four non-receptor tyrosine kinases Tyk2, JAK1, JAK2, and JAK3, which play a critical role in cytokine- and growth factor mediated signal transduction. Cytokine and/or growth factor binding to cell-surface receptor(s), promotes receptor dimerization and facilitates activation of receptor-associated JAK by autophosphorylation. Activated JAK phosphorylates the receptor, creating docking sites for SH2 domain-containing signaling proteins, in particular the STAT family of proteins (STAT1, 2, 3, 4, 5a, 5b and 6). Receptor-bound STATs are themselves phosphorylated by JAKs, promoting their dissociation from the receptor, and subsequent dimerization and translocation to the nucleus. Once in the nucleus, the STATs bind DNA and cooperate with other transcription factors to regulate expression of a number of genes including, but not limited to, genes encoding apoptosis inhibitors (e.g. Bcl-XL, Mcl-1) and cell cycle regulators (e.g. Cyclin D1/D2, c-myc) (Haura et al., Nature Clinical Practice Oncology, 2005, 2(6), 315-324; Verna et al., Cancer and Metastasis Reviews, 2003, 22, 423-434).

Over the past decade, a considerable amount of scientific literature linking constitutive JAK and/or STAT signaling with hyperproliferative disorders and cancer has been published. Constitutive activation of the STAT family, in particular STAT3 and STAT5, has been detected in a wide range of cancers and hyperproliferative disorders (Haura et al., Nature Clinical Practice Oncology, 2005, 2(6), 315-324). Furthermore, aberrant activation of the JAK/STAT pathway provides an important proliferative and/or anti-apoptotic drive downstream of many kinases (e.g. Flt3, EGFR) whose constitutive activation have been implicated as key drivers in a variety of cancers and hyperproliferative disorders (Tibes et al., Annu Rev Pharmacol Toxicol 2550, 45, 357-384; Choudhary et al., International Journal of Hematology 2005, 82(2), 93-99; Sordella et al., Science 2004, 305, 1163-1167). In addition, impairment of negative regulatory proteins, such as the suppressors of cytokine signaling (SOCS) proteins, can also influence the activation status of the JAK/STAT signaling pathway in disease (J C Tan and Rabkin R, Pediatric Nephrology 2005, 20, 567-575).

Several mutated forms of JAK2 have been identified in a variety of disease settings. For example, translocations resulting in the fusion of the JAK2 kinase domain with an oligomerization domain, TEL-JAK2, Bcr-JAK2 and PCM1-JAK2, have been implicated in the pathogenesis of various hematologic malignancies (S D Turner and Alesander D R, Leukemia, 2006, 20, 572-582). More recently, a unique acquired mutation encoding a valine-to-phenylalanine (V617F) substitution in JAK2 was detected in a significant number of polycythemia vera, essential thrombocythemia and idiopathic myelofibrosis patients and to a lesser extent in several other diseases. The mutant JAK2 protein is able to activate downstream signaling in the absence of cytokine stimulation, resulting in autonomous growth and/or hypersensitivity to cytokines and is believed to play a role in driving these diseases (M J Percy and McMullin M F, Hematological Oncology 2005, 23(3-4), 91-93).

### SUMMARY OF THE INVENTION

The present invention relates to compounds of Formula (I):

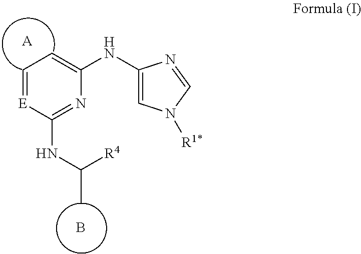

and pharmaceutically acceptable salts thereof.

It is expected that typical compounds of Formula (I) possess beneficial efficacious, metabolic, pharmacokinetic, and/or pharmacodynamic properties.

The compounds of Formula (I) are believed to possess JAK kinase inhibitory activity and are accordingly useful for their anti-proliferation and/or pro-apoptotic activity and in methods of treatment of the human or animal body. The invention also relates to processes for the manufacture of said compound, or pharmaceutically acceptable salts thereof, to pharmaceutical compositions containing it and to its use in the manufacture of medicaments for use in the production of an anti-proliferation and/or pro-apoptotic effect in warm-blooded animals such as man. Also in accordance with the present invention the applicants provide methods of using said compound, or pharmaceutically acceptable salts thereof, in the treatment of myeloproliferative disorders, myelodysplastic syndrome and cancer.

The properties of the compounds of Formula (I) are expected to be of value in the treatment of myeloproliferative disorders, myelodysplastic syndrome, and cancer by inhibiting the tyrosine kinases, particularly the JAK family and more particularly JAK2. Methods of treatment target tyrosine kinase activity, particularly the JAK family activity and more particularly JAK2 activity, which is involved in a variety of myeloproliferative disorders, myelodysplastic syndrome and cancer related processes. Thus, inhibitors of tyrosine kinases, particularly the JAK family and more particularly JAK2, are expected to be active against myeloproliferative disorders such as chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and neoplastic disease such as carcinoma of the breast, ovary, lung, colon, prostate or other tissues, as well as leukemias, myelomas and lymphomas, tumors of the central and peripheral nervous system, and other tumor types such as melanoma, fibrosarcoma and osteosarcoma. Tyrosine kinase inhibitors, particularly the JAK family inhibitors and more particularly JAK2 inhibitors are also expected to be useful for the treatment other proliferative diseases including but not limited to autoimmune, inflammatory, neurological, and cardiovascular diseases.

Furthermore, the compounds of Formula (I), or pharmaceutically acceptable salts thereof, are expected to be of value in the treatment or prophylaxis of against myeloproliferative disorders selected from chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia; particularly myeloma, leukemia, ovarian cancer, breast cancer and prostate cancer.

### DETAILED DESCRIPTION OF THE INVENTION

The present invention relates to compounds of Formula (I):

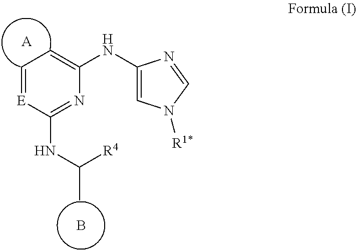

and pharmaceutically acceptable salts thereof, wherein
Ring A is selected from fused 5- or 6-membered heterocycle and fused 5- or 6-membered carbocycle, wherein said fused 5- or 6-membered heterocycle and fused 5- or 6-membered carbocycle are optionally substituted on carbon with one or more R², and wherein if said 5- or 6-membered fused heterocycle contains an —NH— moiety, that —NH— moiety is optionally substituted with R²*;
Ring B is 5- or 6-membered heteroaryl, wherein said 5- or 6-membered heteroaryl is optionally substituted on carbon with one or more R⁵, and wherein if said 5- or 6-membered heteroaryl contains an —NH— moiety, that —NH— moiety is optionally substituted with R⁵*;
E is selected from N and C—R³,
R¹* is selected from H, —CN, C₁₋₆alkyl, carbocyclyl, heterocyclyl, —OR^{1a}, —N(R^{1a})₂, —C(O)H, —C(O)R^{1b}, —C(O)₂R^{1a}, —C(O)N(R^{1a})₂, —S(O)R^{1b}, —S(O)₂R^{1b}, —S(O)₂N(R^{1a})₂, —C(R^{1a})═N(R^{1a}), and —C(R^{1a})═N(OR^{1a}), wherein said C₁₋₆alkyl, carbocyclyl, and heterocyclyl are optionally substituted on carbon with one or more R¹⁰, and wherein if said heterocyclyl contains an —NH— moiety, that —NH— moiety is optionally substituted with R¹⁰*;
R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein any —NH— moiety of said heterocyclyl is optionally substituted with R¹⁰*;
R² in each occurrence is independently selected from halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —N(R^{2a})OR^{2a}, —ON(R^{2a})₂, —C(O)H, —C(O)R^{2b}, —C(O)₂R^{2a}, —C(O)N(R^{2a})₂, —C(O)N(R^{2a})(OR^{2a}) —OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —C(R^{2a})═N(R^{2a}), and —C(R^{2a})═N(OR^{2a}), wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein any —NH— moiety of said heterocyclyl is optionally substituted with R²⁰*;
R²* in each occurrence is independently selected from C₁₋₆alkyl, carbocyclyl, heterocyclyl, —C(O)H, —C(O)R^{2b}, —C(O)₂R^{2a}, —C(O)N(R^{2a})₂, —S(O)R^{2b}, —S(O)₂R^{2b}, —S(O)₂N(R^{2a})₂, —C(R^{2a})═N(R^{2a}), and —C(R^{2a})═N(OR^{2a}), wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein if said heterocyclyl contains an —NH— moiety, that —NH— moiety is optionally substituted with R²⁰*;
R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —N(R^{3a})(OR^{3a}), —O—N(R^{3a})₂, —C(O)H, —C(O)R^{3b}, —C(O)₂R^{3a}, —C(O)N(R^{3a})₂, —C(O)N(R^{3a})(OR^{3a}), —OC(O)N(R^{3a})₂, —N(R^{3a})C(O)₂R³, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —C(R^{3a})═N(R^{3a}), and —C(R^{3a})═N(OR^{3a}), wherein said C₁₋₆alkyl, C₂₋₆alkenyl, C₂₋₆alkynyl, carbocyclyl, and heterocyclyl are optionally substituted on carbon with one or more R³⁰, and wherein if said heterocyclyl contains an —NH— moiety, that —NH— moiety is optionally substituted with R³⁰*;
R^{3a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein if said heterocyclyl contains an —NH— moiety, that —NH— moiety is optionally substituted with R³⁰*;
R^{3b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein if said heterocyclyl contains an —NH— moiety, that —NH— moiety is optionally substituted with R³⁰*;
R⁴ is selected from H, halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —N(R^{4a})(OR^{4a}), —O—N(R^{4a})₂, —C(O)H, —C(O)R^{4b}, —C(O)₂R^{4a}, —C(O)N(R^{4a})₂, —C(O)N(R^{4a})(OR^{4a})—OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S(O)₂R^{4b}, —C(R^{4a})═N(R^{4a}), and —C(R^{4a})═N(OR^{4a}), wherein said C₁₋₆alkyl, C₂₋₆alkenyl, C₂₋₆alkynyl, carbocyclyl, and heterocyclyl are optionally substituted on carbon with one or more R⁴⁰, and wherein if said heterocyclyl contains an —NH— moiety, that —NH— moiety is optionally substituted with R⁴⁰*;
R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein if said heterocyclyl contains an —NH— moiety, that —NH— moiety is optionally substituted with R⁴⁰*;
R^{4b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein if said heterocyclyl contains an —NH— moiety, that —NH— moiety is optionally substituted with R⁴⁰*;
R⁵ in each occurrence is independently selected from H, halo, —CN, C₁₋₆alkyl, C₂₋₆alkenyl, C₂₋₆alkynyl, carbocyclyl, heterocyclyl, —OR^{5a}, —SR^{5a}, —N(R^{5a})₂, —N(R^{5a})C(O)R^{5b}, —N(R^{5a})N(R^{5a})₂, —NO₂, —N(R^{5a})(OR^{5a}), —O—N(R^{5a})₂, —C(O)H, —C(O)R^{5b}, —C(O)₂R^{5a}, —C(O)N(R^{5a})₂, —C(O)N(R^{5a})(OR^{5a})—OC(O)N(R^{5a})₂, —N(R^{5a})C(O)₂R^{5a}, —N(R^{5a})C(O)N(R^{5a})₂, —OC(O)R^{5b}, —S(O)R^{5b}, —S(O)₂R^{5b}, —S(O)₂N(R^{5a})₂, —N(R^{5a})S(O)₂R^{5b}, —C(R^{5a})═N(R^{5a}), and —C(R^{5a})═N(OR^{5a}), wherein said C₁₋₆alkyl, C₂₋₆alkenyl, C₂₋₆alkynyl, carbocyclyl, and heterocyclyl in each occurrence are optionally and independently substituted on carbon with one or more R⁵⁰, and wherein if said heterocyclyl contains an —NH— moiety, that —NH— moiety is optionally substituted with R⁵⁰*;
R⁵* in each occurrence is independently selected from H, —CN, C₁₋₆alkyl, carbocyclyl, heterocyclyl, —OR^{5a}, —N(R^{5a})₂, —C(O)H, —C(O)R^{5b}, —C(O)₂R^{5a}, —C(O)N(R^{5a})₂, —S(O)R^{5b}, —S(O)₂R^{5b}, —S(O)₂N(R^{5a})₂, —C(R^{5a})═N(R^{5a}), and —C(R^{5a})═N(OR^{5a}), wherein said C₁₋₆alkyl, carbocyclyl, and heterocyclyl in each occurrence are optionally and independently substituted on carbon with one or more R⁵⁰, and wherein if said heterocyclyl contains an —NH— moiety, that —NH— moiety is optionally substituted with R⁵⁰*;
R^{5a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁵⁰, and wherein if said heterocyclyl contains an —NH— moiety, that —NH— moiety is optionally substituted with R⁵⁰*;
R^{5b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁵⁰, and wherein if said heterocyclyl contains an —NH— moiety, that —NH— moiety is optionally substituted with R⁵⁰*;
R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})C(O)R^{10b}, —N(R^{10a})N(R^{10a})₂, —NO₂, —N(R^{10a})(OR^{10a}), —O—N(R^{10a})₂, —C(O)H, —C(O)R^{10b}, —C(O)₂R^{10a}, —C(O)N(R^{10a})₂, —C(O)N(R^{10a})(OR^{10a}), —OC(O)N(R^{10a})₂, —N(R^{10a})C(O)₂R^{10a}, —N(R^{10a})C(O)N(R^{10a})₂, —OC(O)R^{10b}, —S(O)R^{10b}, —S(O)₂R^{10b}, —S(O)₂N(R^{10a})₂—N(R^{10a})₂, —N(R^{10a})S(O)₂R^{10b}, —C(R^{10a})═N(R^{10a}), and —C(R^{10a})═N(OR^{10a});
R¹⁰* in each occurrence is independently selected from C₁₋₆alkyl, carbocyclyl, heterocyclyl, —C(O)H, —C(O)R^{10b}, —C(O)₂R^{10a}, —C(O)N(R^{10a})₂, —S(O)R^{10b}, —S(O)₂R^{10b}, —S(O)₂N(R^{10a})₂, —C(R^{10a})═N(R^{10a}), and —C(R^{10a})═N(OR^{10a});
R^{10a} in each occurrence is independently selected from H, C₁₋₆alkyl, carbocyclyl, and heterocyclyl;

R^{10b} in each occurrence is independently selected from C₁₋₆alkyl, C₂₋₆alkenyl, C₂₋₆alkynyl, carbocyclyl, and heterocyclyl;

R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})C(O)R^{20b}, —N(R^{20a})N(R^{20a})₂, —NO₂, —N(R^{20a})—OR^{20a}, —O—N(R^{20a})₂, —C(O)H, —C(O)R^{20b}, —C(O)₂R^{20a}, —C(O)N(R^{20a})₂, —C(O)N(R^{20a})(OR^{20a}), —OC(O)N(R^{20a})₂, —N(R^{20a})C(O)₂R^{20a}, —N(R^{20a})C(O)N(R^{20a})₂, OC(O)R^{20b}, —S(O)R^{20b}, —S(O)₂R^{20b}, —S(O)₂N(R^{20a})₂, —N(R^{20a})S(O)₂R^{20b}, —C(R^{20a})═N(R^{20a}), and —C(R^{20a})═N(OR^{20a}), wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R²⁰* in each occurrence is independently selected from —CN, C₁₋₆alkyl, carbocyclyl, heterocyclyl, —OR^{20a}, —N(R^{20a})₂, —C(O)H, —C(O)R^{20b}, —C(O)₂R^{20a}, —C(O)N(R^{20a})₂, —S(O)R^{20b}, —S(O)₂R^{20b}, —S(O)₂N(R^{20a})₂, —C(R^{20a})═N(R^{20a}) and —C(R^{20a})═N(OR^{20a}), wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —N(R^{30a})(OR^{30a}), —O—N(R^{30a})₂, —C(O)H, —C(O)R^{30b}, —C(O)₂R^{30a}, —C(O)N(R^{30a})₂, —C(O)N(R^{30a})(OR^{30a}), —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —C(R^{30a})═N(R^{30a}), and —C(R^{30a})═N(OR^{30a});
R³⁰* in each occurrence is independently selected from —CN, C₁₋₆alkyl, carbocyclyl, heterocyclyl, —OR^{30a}, —N(R^{30a})₂, —C(O)H, —C(O)R^{30b}, —C(O)₂R^{30a}, —C(O)N(R^{30a})₂, —S(O)R^{30b}, —S(O)₂R^{30b}, —S(O)₂N(R^{30a})₂, —C(R^{30a})═N(R^{30a}), and —C(R^{30a})═N(OR^{30a});
R^{30a} in each occurrence is independently selected from H, C₁₋₆alkyl, carbocyclyl, and heterocyclyl;
R^{30b} in each occurrence is independently selected from C₁₋₆alkyl, C₂₋₆alkenyl, C₂₋₆alkynyl, carbocyclyl, and heterocyclyl;
R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})C(O)R^{40b}, —N(R^{40a})N(R^{40a})₂, —NO₂, —N(R^{40a})(OR^{40a}), —O—N(R^{40a})₂, —C(O)H, —C(O)R^{40b}, —C(O)₂R^{40a}, —C(O)N(R^{40a})₂, —C(O)N(R^{40a})(OR^{40a}), —OC(O)N(R^{40a})₂, —N(R^{40a})C(O)₂R^{40a}, —N(R^{40a})C(O)N(R^{40a})₂, —OC(O)R^{40b}, —S(O)R^{40b}, —S(O)₂R^{40b}, —S(O)₂N(R^{40a})₂, —N(R^{40a})S(O)₂R^{40b}, —C(R^{40a})═N(R^{40a}), and —C(R^{40a})═N(OR^{40a});
R⁴⁰* in each occurrence is independently selected from —CN, C₁₋₆alkyl, carbocyclyl, heterocyclyl, —OR^{40a}, —N(R^{40a})₂, —C(O)H, —C(O)R^{40b}, —C(O)₂R^{40a}, —C(O)N(R^{40a})₂, —S(O)R^{40b}, —S(O)₂R^{40b}, —S(O)₂N(R^{40a})₂, —C(R^{40a})═N(R^{40a}), and —C(R^{40a})═N(OR^{40a});
R^{40a} in each occurrence is independently selected from H, C₁₋₆alkyl, carbocyclyl, and heterocyclyl;
R^{40b} in each occurrence is independently selected from C₁₋₆alkyl, C₂₋₆alkenyl, C₂₋₆alkynyl, carbocyclyl, and heterocyclyl;
R⁵⁰ in each occurrence is independently selected from halo, —CN, C₁₋₆alkyl, C₂₋₆alkenyl, C₂₋₆alkynyl, carbocyclyl, heterocyclyl, —OR^{50a}, —SR^{50a}, —N(R^{50a})₂, —N(R^{50a})C(O)R^{50b}, —N(R^{50a})N(R^{50a})₂, —NO₂, —N(R^{50a})(OR^{50a}), —O—N(R^{50a})₂, —C(O)H, —C(O)R^{50b}, —C(O)₂R^{50a}, —C(O)N(R^{50a})₂, —C(O)N(R^{50a})(OR^{50a}), —OC(O)N(R^{50a})₂, —N(R^{50a})C(O)₂R^{50a}, —N(R^{50a})C(O)N(R^{50a})₂, —OC(O)R^{50b}, —S(O)R^{50b}, —S(O)₂R^{50b}, —S(O)₂N(R^{50a})₂, —N(R^{50a})S(O)₂R^{50b}, —C(R^{50a})═N(R^{50a}), and —C(R^{50a})═N(OR^{50a});
R⁵⁰* in each occurrence is independently selected from —CN, C₁₋₆alkyl, carbocyclyl, heterocyclyl, —OR^{50a}, —N(R^{50a})₂, —C(O)H, —C(O)R^{50b}, —C(O)₂R^{50a}, —C(O)N(R^{50a})₂, —S(O)R^{50b}, —S(O)₂R^{50b}, —S(O)₂N(R^{50a})₂, —C(R^{50a})═N(R^{50a}), and —C(R^{50a})═N(OR^{50a});
R^{50a} in each occurrence is independently selected from H, C₁₋₆alkyl, carbocyclyl, and heterocyclyl;
R^{50b} in each occurrence is independently selected from C₁₋₆alkyl, C₂₋₆alkenyl, C₂₋₆alkynyl, carbocyclyl, and heterocyclyl;
R^{b} in each occurrence is independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂, —N(R^{m})C(O)Rⁿ, —N(R^{m})N(R^{m})₂, —NO₂, —N(R^{m})—OR^{m}, —O—N(R^{m})₂, —C(O)H, —C(O)Rⁿ, —C(O)₂R^{m}, —C(O)N(R^{m})₂, —C(O)N(R^{m})(OR^{m}), —OC(O)N(R^{m})₂, —N(R^{m})C(O)₂R^{m}, —N(R^{m})C(O)N(R^{m})₂, —OC(O)Rⁿ, —S(O)Rⁿ, —S(O)₂Rⁿ, —S(O)₂N(R^{m})₂, —N(R^{m})S(O)₂Rⁿ, —C(R^{m})═N(R^{m}), and —C(R^{m})═N(OR^{m});
R^{b}* in each occurrence is independently selected from —CN, C₁₋₆alkyl, carbocyclyl, heterocyclyl, —OR^{m}, —N(R^{m})₂, —C(O)H, —C(O)Rⁿ, —C(O)₂R^{m}, —C(O)N(R^{m})₂, —S(O)Rⁿ, —S(O)₂Rⁿ, —S(O)₂N(R^{m})₂, —C(R^{m})═N(R^{m}), and —C(R^{m})═N(OR^{m});
R^{m} in each occurrence is independently selected from H, C₁₋₆alkyl, carbocyclyl, and heterocyclyl; and
Rⁿ in each occurrence is independently selected from C₁₋₆alkyl, C₂₋₆alkenyl, C₂₋₆alkynyl, carbocyclyl, and heterocyclyl.

In this specification the prefix C_{x-y} as used in terms such as C_{x-y}alkyl and the like (where x and y are integers) indicates the numerical range of carbon atoms that are present in the group; for example, C₁₋₄alkyl includes C₁alkyl (methyl), C₂alkyl (ethyl), C₃alkyl (propyl and isopropyl) and C₄alkyl (butyl, 1-methylpropyl, 2-methylpropyl, and t-butyl).

Alkyl—As used herein the term “alkyl” refers to both straight and branched chain saturated hydrocarbon radicals having the specified number of carbon atoms. References to individual alkyl groups such as “propyl” are specific for the straight chain version only and references to individual branched chain alkyl groups such as ‘isopropyl’ are specific for the branched chain version only.

Alkenyl—As used herein, the term “alkenyl” refers to both straight and branched chain hydrocarbon radicals having the specified number of carbon atoms and containing at least one carbon-carbon double bond. For example, “C₂₋₆alkenyl” includes, but is not limited to, groups such as C₂₋₅alkenyl, C₂₋₄alkenyl, ethenyl, 2-propenyl, 2-methyl-2-propenyl, 3-butenyl, 4-pentenyl, and 5-hexenyl.

Alkynyl—As used herein, the term “alkynyl” refers to both straight and branched chain hydrocarbon radicals having the specified number of carbon atoms and containing at least one carbon-carbon triple bond. For example, “C₂₋₆alkynyl” includes, but is not limited to, groups such as C₂₋₅alkynyl, C₂₋₄alkynyl, ethynyl, 2-propynyl, 2-methyl-2-propynyl, 3-butynyl, 4-pentynyl, and 5-hexynyl.

Halo—As used herein, the term “halo” refers to fluoro, chloro, bromo and iodo. In one aspect, the term “halo” may refer to fluoro, chloro, and bromo. In another aspect, the term “halo” may refer to fluoro and chloro. In still another aspect, the term “halo” may refer to fluoro.

Carbocyclyl—As used herein, the term “carbocyclyl” refers to a saturated, partially saturated, or unsaturated, mono or bicyclic carbon ring that contains 3 to 12 ring atoms, of which one or more —CH₂— groups may be optionally replaced with a corresponding number of —C(O)— groups. Illustrative examples of “carbocyclyl” include, but are not limited to, adamantyl, cyclopropyl, cyclobutyl, cyclopentyl, cyclopentenyl, cyclohexyl, cyclohexenyl, indanyl, naphthyl, oxocyclopentyl, 1-oxoindanyl, phenyl, and tetralinyl.

3- to 6-Membered Carbocyclyl—In one aspect, “carbocyclyl” may be “3- to 6-membered carbocyclyl.” As used herein, the term “3- to 6-membered carbocyclyl” refers to a saturated, partially saturated, or unsaturated monocyclic carbon ring containing 3 to 6 ring atoms, of which one or more —CH₂— groups may be optionally replaced with a corresponding number of —C(O)— groups. Illustrative examples of “3- to 6-membered carbocyclyl” include cyclopropyl, cyclobutyl, cyclopentyl, oxocyclopentyl, cyclopentenyl, cyclohexyl, and phenyl.

3- to 5-Membered Carbocyclyl—In one aspect, “carbocyclyl” and “3- to 6-membered carbocyclyl” may be “3- to 5-membered carbocyclyl.” The term “3- to 5-membered carbocyclyl” refers to a saturated or partially saturated monocyclic carbon ring containing 3 to 5 ring atoms, of which one or more —CH₂— groups may be optionally replaced with a corresponding number of —C(O)— groups. Illustrative examples of “3- to 5-membered carbocyclyl” include cyclopropyl, cyclobutyl, cyclopentyl, oxocyclopentyl, and cyclopentenyl. In one aspect, “3- to 5-membered carbocyclyl” may be cyclopropyl.

Fused 5- or 6-Membered Carbocycle—For the purposes of Ring A, the term “fused 5- or 6-membered carbocycle” is intended to refer to a monocyclic carbon ring containing 5 or 6 ring atoms of which one or more —CH₂— groups may be optionally replaced with a corresponding number of —C(O)— groups. The fused 5- or 6-membered carbocycle shares two adjacent carbon atoms with the ring (pyridine when E is carbon, and pyrimidine when E is nitrogen) to which it is fused, forming a bicyclic ring system. Illustrative examples of the term “fused 5- or 6-membered carbocycle” include fused cyclopentane, fused cyclohexane, fused benzene, and fused oxocyclopentane. In aspect, “fused 5- or 6-membered carbocycle” may refer to fused cyclopentane. In another aspect, “fused 5- or 6-membered carbocycle” may refer to fused benzene.

For example, an embodiment of Formula (I) in which Ring A is unsubstituted fused cyclopentane would have the following structure:

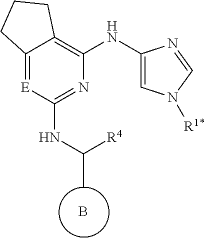

Fused 5-Membered Carbocycle—In one aspect, “fused 5- or 6-membered carbocycle” may be “fused 5-membered carbocycle.” The term “fused 5-membered carbocycle” is intended to refer to a monocyclic carbon ring containing 5 ring atoms of which one or more —CH₂— groups may be optionally replaced with a corresponding number of —C(O)— groups. The fused 5-membered carbocycle shares two adjacent carbon atoms with the ring (pyridine when E is carbon, and pyrimidine when E is nitrogen) to which it is fused, forming a bicyclic ring system. Illustrative examples of the term “fused 5-membered carbocycle” include fused cyclopentane and fused oxocyclopentane.

Heterocyclyl—As used herein, the term “heterocyclyl” refers to a saturated, partially saturated, or unsaturated, mono or bicyclic ring containing 4 to 12 ring atoms of which at least one ring atom is selected from nitrogen, sulfur, and oxygen, and which may, unless otherwise specified, be carbon or nitrogen linked, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of the term “heterocyclyl” include, but are not limited to, 1,3-benzodioxolyl, 3,5-dioxopiperidinyl, furanyl, imidazolyl, indolyl, isoquinolinyl, isothiazolyl, isoxazolyl, morpholinyl, 2-oxa-5-azabicyclo[2.2.1]hept-5-yl, oxazolyl, 2-oxopyrrolidinyl, 2-oxo-1,3-thiazolidinyl, piperazinyl, piperidyl, 2H-pyranyl, pyrazolyl, pyridinyl, pyrrolyl, pyrrolidinyl, pyrrolidinyl, pyrimidinyl, pyrazinyl, pyrazolyl, pyridazinyl, quinolyl, tetrahydrofuranyl, tetrahydropyranyl, thiazolyl, thiadiazolyl, thiazolidinyl, thiomorpholinyl, thiophenyl, pyridine-N-oxidyl and quinoline-N-oxidyl.

4- to 6-Membered Heterocyclyl—The term “4- to 6-membered heterocyclyl” refers to a saturated, partially saturated, or unsaturated, monocyclic ring containing 4 to 6 ring atoms, of which at least one ring atom is selected from nitrogen, sulfur, and oxygen, and of which a —CH₂— group may be optionally replaced by a —C(O)— group. Unless otherwise specified, “4- to 6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “4- to 6-membered heterocyclyl” include azetidin-1-yl, dioxidotetrahydrothiophenyl, 2,4-dioxoimidazolidinyl, 3,5-dioxopiperidinyl, furanyl, imidazolyl, isothiazolyl, isoxazolyl, morpholinyl, oxazolyl, oxetanyl, oxoimidazolidinyl, 3-oxo-1-piperazinyl, 2-oxopyrrolidinyl, 2-oxotetrahydrofuranyl, oxo-1,3-thiazolidinyl, piperazinyl, piperidyl, 2H-pyranyl, pyrazolyl, pyridinyl, pyrrolyl, pyrrolidinyl, pyrimidinyl, pyrazinyl, pyrazolyl, pyridazinyl, tetrahydrofuranyl, tetrahydropyranyl, thiazolyl, 1,3,4-thiadiazolyl, thiazolidinyl, thiomorpholinyl, thiophenyl, 4H-1,2,4-triazolyl, and pyridine-N-oxidyl.

5- or 6-Membered Heterocyclyl—In one aspect, “heterocyclyl” and “4- to 6-membered heterocyclyl” may be “5- or 6-membered heterocyclyl.” The term “5- or 6-membered heterocyclyl” refers to a saturated, partially saturated, or unsaturated, monocyclic ring containing 5 or 6 ring atoms, of which at least one ring atom is selected from nitrogen, sulfur, and oxygen, and of which a —CH₂— group may be optionally replaced by a —C(O)— group. Unless otherwise specified, “5- or 6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heterocyclyl” include dioxidotetrahydrothiophenyl, 2,4-dioxoimidazolidinyl, 3,5-dioxopiperidinyl, furanyl, imidazolyl, isothiazolyl, isoxazolyl, morpholinyl, oxazolyl, oxoimidazolidinyl, 3-oxo-1-piperazinyl, 2-oxopyrrolidinyl, 2-oxotetrahydrofuranyl, oxo-1,3-thiazolidinyl, piperazinyl, piperidyl, 2H-pyranyl, pyrazolyl, pyridinyl, pyrrolyl, pyrrolidinyl, pyrimidinyl, pyrazinyl, pyrazolyl, pyridazinyl, tetrahydrofuranyl, tetrahydropyranyl, thiazolyl, 1,3,4-thiadiazolyl, thiazolidinyl, thiomorpholinyl, thiophenyl, 4H-1,2,4-triazolyl, and pyridine-N-oxidyl.

6-Membered Heterocyclyl—In one aspect, “heterocyclyl,” “4- to 6-membered heterocyclyl,” and “5- or 6-membered heterocyclyl” may be “6-membered heterocycyl.” As used herein, the term “6-membered heterocyclyl” refers to a saturated, partially saturated, or unsaturated, monocyclic ring containing 6 ring atoms, of which at least one ring atom is selected from nitrogen, sulfur, and oxygen, and of which a —CH₂— group may be optionally replaced by a —C(O)— group. Unless otherwise specified, “6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “6-membered heterocyclyl” include, but are not limited to, 3,5-dioxopiperidinyl, morpholinyl, piperazinyl, piperidinyl, 2H-pyranyl, pyrazinyl, pyridazinyl, pyridinyl, and pyrimidinyl.

5- or 6-Membered Heteroaryl—In one aspect, “heterocyclyl,” “4- to 6-membered heterocyclyl,” and “5- or 6-membered heterocyclyl” may be “5- or 6-membered heteroaryl.” As used herein, the term “5- or 6-membered heteroaryl” is intended to refer to a monocyclic, aromatic heterocyclyl ring containing 5 or 6 ring atoms, of which at least one ring atom is selected from nitrogen, sulfur, and oxygen. Unless otherwise specified, “6-membered heteroaryl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heteroaryl” include furanyl, imidazolyl, isothiazolyl, isoxazole, oxazolyl, pyrazinyl, pyrazolyl, pyridazinyl, pyrimidinyl, pyridinyl, pyrrolyl, 1,3,4-thiadiazolyl, thiazolyl, thiophenyl, and 4H-1,2,4-triazolyl.

6-Membered Heteroaryl—In one aspect, “heterocyclyl”, “4- to 6-membered heterocyclyl,” “5- or 6-membered heterocyclyl,” “6-membered heterocyclyl,” and “5- or 6-membered heteroaryl” may be “6-membered heteroaryl.” As used herein, the term “6-membered heteroaryl” is intended to refer to a monocyclic, aromatic heterocyclyl ring containing 6 ring atoms. Unless otherwise specified, “6-membered heteroaryl” groups may be carbon or nitrogen linked. Ring nitrogen atoms may be optionally oxidized to form an N-oxide. Illustrative examples of the term “6-membered heteroaryl” include, but are not limited to, pyrazinyl, pyridazinyl, pyrimidinyl, and pyridinyl.

Fused 5- or 6-Membered Heterocycle—For the purposes of Ring A, the term “fused 5- or 6-membered heterocycle” is intended to refer to a monocyclic ring containing 5 or 6 ring atoms of which at least one ring atom is selected from nitrogen, sulfur, and oxygen. The 5- or 6-membered heterocycle shares two carbon atoms with the ring (pyridine when E is carbon, and pyrimidine when E is nitrogen) to which it is fused, forming a bicyclic ring system. Ring sulfur atoms may be optionally oxidized to form S-oxides. Ring nitrogen atoms may be optionally oxidized to form N-oxides. Illustrative examples of the term “fused 5- or 6-membered heterocycle” include fused furan, fused imidazole, fused isothiazole, fused isoxazole, fused morpholine, fused oxadiazole, fused oxazole, 2-oxopyrrolidine, fused piperazine, fused piperidine, fused pyran, fused pyrazine, fused pyrazole, fused pyridazine, fused pyridine, fused pyrimidine, fused pyrrole, fused pyrrolidine, fused tetrahydrofuran, fused tetrahydropyran, fused thiazole, fused thiophene, fused thiadiazole, and fused triazole.

For example, an embodiment of Formula (I) in which Ring A is unsubstituted fused pyrrole would encompass the following structures:

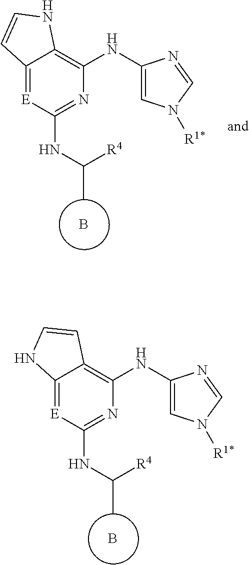

Fused 5-Membered Heterocycle—In one aspect “fused 5- or 6-membered heterocycle” may be “fused 5-membered heterocycle.” The term “fused 5-membered heterocycle” is intended to refer to a monocyclic ring containing 5 ring atoms of which at least one ring atom is selected from nitrogen, sulfur, and oxygen. The 5-membered heterocycle shares two carbon atoms with the ring (pyridine when E is carbon, and pyrimidine when E is nitrogen) to which it is fused, forming a bicyclic ring system. Ring sulfur atoms may be optionally oxidized to form S-oxides. Ring nitrogen atoms may be optionally oxidized to form N-oxides. Illustrative examples of the term “fused 5-membered heterocycle” include fused furan, fused imidazole, fused isothiazole, fused isoxazole, fused oxadiazole, fused oxazole, 2-oxopyrrolidine, fused pyrazole, fused pyrrole, fused pyrrolidine, fused tetrahydrofuran, fused thiazole, fused thiophene, fused thiadiazole, and fused triazole.

Fused 6-Membered Heterocycle—In one aspect “fused 5- or 6-membered heterocycle” may be “fused 6-membered heterocycle.” The term “fused 6-membered heterocycle” is intended to refer to a monocyclic ring containing 6 ring atoms of which at least one ring atom is selected from nitrogen, sulfur, and oxygen. The 6-membered heterocycle shares two carbon atoms with the ring (pyridine when E is carbon, and pyrimidine when E is nitrogen) to which it is fused, forming a bicyclic ring system. Ring sulfur atoms may be optionally oxidized to form S-oxides. Ring nitrogen atoms may be optionally oxidized to form N-oxides. Illustrative examples of the term “fused 5-membered heterocycle” include fused pyrazine and fused pyridine.

Where a particular R group (e.g. R^{1a}, R¹⁰, etc.) is present in a compound of Formula (I) more than once, it is intended that each selection for that R group is independent at each occurrence of any selection at any other occurrence. For example, the —N(R)₂ group is intended to encompass: 1) those —N(R)₂ groups in which both R substituents are the same, such as those in which both R substituents are, for example, C₁₋₆alkyl; and 2) those —N(R)₂ groups in which each R substituent is different, such as those in which one R substituent is, for example, H, and the other R substituent is, for example, carbocyclyl.

Unless specifically stated, the bonding atom of a group may be any suitable atom of that group; for example, propyl includes prop-1-yl and prop-2-yl.

Effective Amount—As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

In particular, an effective amount of a compound of Formula (I) for use in the treatment of cancer is an amount sufficient to symptomatically relieve in a warm-blooded animal such as man, the symptoms of cancer and myeloproliferative diseases, to slow the progression of cancer and myeloproliferative diseases, or to reduce in patients with symptoms of cancer and myeloproliferative diseases the risk of getting worse.

Leaving Group—As used herein, the phrase “leaving group” is intended to refer to groups readily displaceable by a nucleophile such as an amine nucleophile, and alcohol nucleophile, or a thiol nucleophile. Examples of suitable leaving groups include halo, such as chloro and bromo, and sulfonyloxy group, such as methanesulfonyloxy and toluene-4-sulfonyloxy.

Optionally substituted—As used herein, the phrase “optionally substituted,” indicates that substitution is optional and therefore it is possible for the designated group to be either substituted or unsubstituted. In the event a substitution is desired, any number of hydrogens on the designated group may be replaced with a selection from the indicated substituents, provided that the normal valency of the atoms on a particular substituent is not exceeded, and that the substitution results in a stable compound.

In one aspect, when a particular group is designated as being optionally substituted with “one or more” substituents, the particular may be unsubstituted. In another aspect, the particular group may bear one substituent. In another aspect, the particular substituent may bear two substituents. In still another aspect, the particular group may bear three substituents. In yet another aspect, the particular group may bear four substituents. In a further aspect, the particular group may bear one or two substituents. In still a further aspect, the particular group may be unsubstituted, or may bear one or two substituents.

Pharmaceutically Acceptable—As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Protecting Group—As used herein, the term “protecting group” is intended to refer to those groups used to prevent selected reactive groups (such as carboxy, amino, hydroxy, and mercapto groups) from undergoing undesired reactions.

Illustrative examples of suitable protecting groups for a hydroxy group include, but are not limited to, an acyl group; alkanoyl groups such as acetyl; aroyl groups, such as benzoyl; silyl groups, such as trimethylsilyl; and arylmethyl groups, such as benzyl. The deprotection conditions for the above hydroxy protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

Illustrative examples of suitable protecting groups for an amino group include, but are not limited to, acyl groups; alkanoyl groups such as acetyl; alkoxycarbonyl groups, such as methoxycarbonyl, ethoxycarbonyl, and t-butoxycarbonyl; arylmethoxycarbonyl groups, such as benzyloxycarbonyl; and aroyl groups, such benzoyl. The deprotection conditions for the above amino protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron trichloride). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or work-up.

With reference to substituent R¹ for illustrative purposes, the following substituent definitions have the indicated structures:

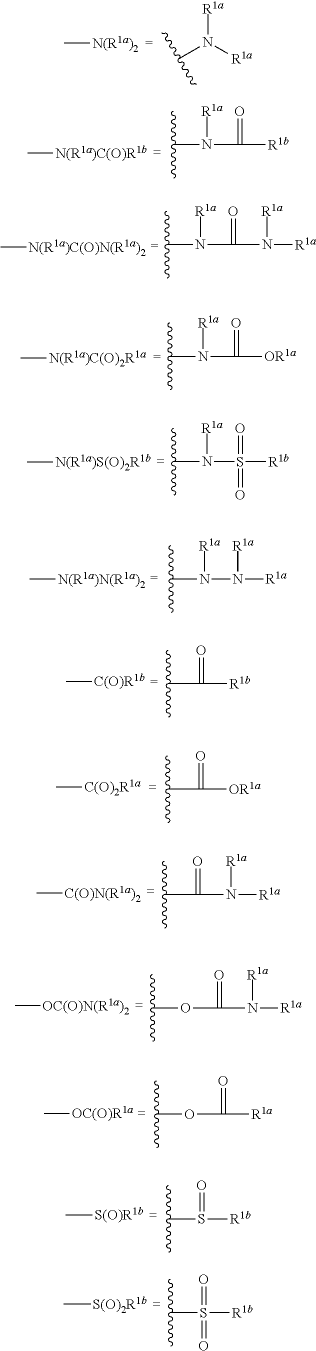

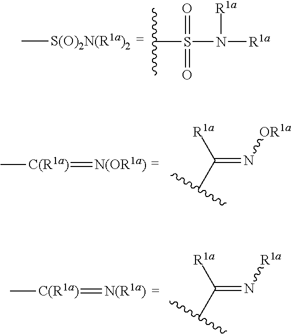

The compounds discussed herein in many instances were named and/or checked with ACD/Name (ACD/Labs Release: 10.00, Product Version 10.04 (Build 18136, 22 Mar. 2007) by ACD/Labs®.

Compounds of Formula (I) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethyl-sulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N-methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Compounds of Formula (I) have one or more chiral centers and/or geometric isomeric centers (E- and Z-isomers), and it is to be understood that the invention encompasses all such optical, diastereoisomers and geometric isomers. The invention further relates to any and all tautomeric forms of the compounds of Formula (I).

It is also to be understood that certain compounds of Formula (I) can exist in solvated as well as unsolvated forms such as, for example, hydrated forms. It is to be understood that the invention encompasses all such solvated forms.

Additional embodiments of the invention are as follows. These additional embodiments relate to compounds of Formula (I) and pharmaceutically acceptable salts thereof. Such specific substituents may be used, where appropriate, with any of the definitions, claims or embodiments defined hereinbefore or hereinafter.

### Ring A

In one aspect, Ring A is selected from fused 5- or 6-membered heterocycle and fused 5- or 6-membered carbocycle, wherein said fused 5- or 6-membered heterocycle and fused 5- or 6-membered carbocycle are optionally substituted on carbon with one or more R², and wherein any —NH— moiety of said fused 5- or 6-membered heterocycle is optionally substituted with R²*;

R² in each occurrence is independently selected from halo, C₁₋₆alkyl, 5- or 6-membered heterocyclyl, —OR^{2a}, and —N(R^{2a})₂, wherein said C₁₋₆alkyl is optionally substituted with one or more R²⁰;
R²* in each occurrence is independently selected from C₁₋₆alkyl and 3- to 5-membered carbocyclyl, wherein said C₁₋₆alkyl is optionally substituted with one or more R²⁰;
R^{2a} in each occurrence is independently selected from H, C₁₋₆alkyl, and 3- to 5-membered carbocyclyl; and
R^{2b} in each occurrence is independently selected from halo and —OH.

In another aspect, Ring A is fused 5- or 6-membered heterocycle, wherein said fused 5- or 6-membered heterocycle is optionally substituted on carbon with one or more R², and wherein any —NH— moiety of said fused 5- or 6-membered heterocycle is optionally substituted with R²*;

R² is selected from halo, C₁₋₆alkyl, 5- or 6-membered heterocyclyl, and —N(R^{2a})₂, wherein said C₁₋₆alkyl is optionally substituted with one or more R²⁰;
R²* is selected from C₁₋₆alkyl and 3- to 5-membered carbocyclyl, wherein said C₁₋₆alkyl is optionally substituted with one or more R²⁰;
R^{2a} in each occurrence is independently selected from H and 3- to 5-membered carbocyclyl; and
R²⁰ in each occurrence is independently selected from halo and —OH.

In still another aspect, Ring A is selected from fused 5-membered heterocycle and fused 5-membered carbocycle, wherein said fused 5-membered heterocycle and fused 5-membered carbocycle are optionally substituted on carbon with one or more R², and wherein any —NH— moiety of said fused 5-membered heterocycle is optionally substituted with R²*;

R² is C₁₋₆alkyl;

### R²* is —S(O)₂R^{2b};

R^{2b} is phenyl, wherein said phenyl is optionally substituted with one or more R²⁰; and
R²⁰ is C₁₋₆alkyl.

In yet another aspect, Ring A is fused 6-membered heterocycle, wherein said fused 6-membered heterocycle is optionally substituted on carbon with one or more R²;

R² in each occurrence is independently selected from halo, C₁₋₆alkyl, 5- or 6-membered heterocyclyl, —OH, and —N(R^{2a})₂, wherein said C₁₋₆alkyl in each occurrence is optionally and independently substituted with one or more R²⁰;
R^{2a} in each occurrence is independently selected from H and 3- to 5-membered carbocyclyl; and
R²⁰ is halo.

In a further aspect, Ring A is fused 5-membered heterocycle, wherein said fused 5-membered heterocycle is optionally substituted on carbon with one or more R², and wherein any —NH—moiety of said fused 5-membered heterocycle is optionally substituted with R²*;

R² is C₁₋₆alkyl, wherein said C₁₋₆alkyl is optionally substituted with halo;
R²* in each occurrence is independently selected from C₁₋₆alkyl and 3- to 5-membered carbocyclyl, wherein said C₁₋₆alkyl is optionally substituted on carbon with one or more R²⁰;
R²⁰ in each occurrence is independently selected from halo and —OH.

In still a further aspect, Ring A is fused 5-membered heterocycle, wherein said fused 5-membered heterocycle is optionally substituted on carbon with one or more R², and wherein any —NH— moiety of said fused 5-membered heterocycle is optionally substituted with R²*;

R² is C₁₋₆alkyl;

### R²* is —S(O)₂R^{2b};

R^{2b} is phenyl, wherein said phenyl is optionally substituted with one or more R²⁰; and
R²⁰ is C₁₋₆alkyl.

In yet a further aspect Ring A is fused 5- or 6-membered carbocycle, wherein said fused 5- or 6-membered carbocycle is optionally substituted with one or more R²;

### R² is —OR^{2a};

R^{2a} is C₁₋₆alkyl.

In one aspect, Ring A is selected from fused pyrazole, fused pyridine, fused pyrrole, fused thiazole, and fused thiophene, wherein said fused pyrazole, fused pyridine, fused pyrrole, fused thiazole, and fused thiophene are optionally substituted on carbon with one or more R²; and wherein the —NH— moiety of said fused pyrrole and fused pyrazole is optionally substituted with R²*;

R² in each occurrence is independently selected from halo, C₁₋₆alkyl, morpholin-4-yl, —OH, and —N(R^{2a})₂, wherein said C₁₋₆alkyl in each occurrence is optionally substituted with halo;
R²* is selected from C₁₋₆alkyl and 3- to 5-membered carbocyclyl, wherein said C₁₋₆alkyl is optionally substituted with one or more R²⁰;
R^{2a} in each occurrence is independently selected from H and 3- to 5-membered carbocyclyl;
R²⁰ in each occurrence is independently selected from halo and —OH.

In another aspect, Ring A is selected from fused cyclopentane, fused pyrrole, fused thiazole, and fused thiophene, wherein said fused cyclopentane, fused pyrrole, fused thiazole, and fused thiophene are optionally substituted on carbon with one or more R², and wherein the —NH— moiety of said fused pyrrole is optionally substituted with R²*;

R² is C₁₋₆alkyl;

### R²* is —S(O)₂R^{2b};

R^{2b} is phenyl, wherein said phenyl is optionally substituted with one or more R²⁰; and
R²⁰ is C₁₋₆alkyl.

In still another aspect, Ring A is selected from fused cyclopentane, fused pyrrole, fused thiazole, and fused thiophene, wherein said fused cyclopentane, fused pyrrole, fused thiazole, and fused thiophene are optionally substituted on carbon with one or more R², and wherein the —NH— moiety of said fused pyrrole is optionally substituted with R²*;

R² is methyl;

### R²* is —S(O)₂R^{2b};

R^{2b} is phenyl, wherein said phenyl is optionally substituted with one or more R²⁰; and
R²⁰ is methyl.
Ring A, Together with the Pyrimidine to which it is Fused, and E

In one aspect, Ring A, together with the pyrimidine to which it is fused, forms a member selected from 6,7-dihydro-5H-cyclopenta[d]pyrimidine, 5H-pyrrolo[3,2-d]pyrimidine, 7H-pyrrolo[2,3-d]pyrimidine, [1,3]thiazolo[5,4-d]pyrimidine, thieno[2,3-d]pyrimidine, and thieno[3,2-d]pyrimidine, wherein said 6,7-dihydro-5H-cyclopenta[d]pyrimidine, 5H-pyrrolo[3,2-d]pyrimidine, 7H-pyrrolo[2,3-d]pyrimidine, [1,3]thiazolo[5,4-d]pyrimidine, thieno[2,3-d]pyrimidine, thieno[3,2-d]pyrimidine are optionally substituted on carbon with one or more R², and wherein any —NH— moiety of said 5H-pyrrolo[3,2-d]pyrimidine, and 7H-pyrrolo[2,3-d]pyrimidine is optionally substituted with R²*;

### E is N;

R² is C₁₋₆alkyl;

### R²* is —S(O)₂R^{2b};

R^{2b} is phenyl, wherein said phenyl is optionally substituted with one or more R²⁰;
R²⁰ is C₁₋₆alkyl.

In another aspect, Ring A, together with the pyrimidine to which it is fused, forms a member selected from 6,7-dihydro-5H-cyclopenta[d]pyrimidine, 5H-pyrrolo[3,2-d]pyrimidine, 7H-pyrrolo[2,3-d]pyrimidine, [1,3]thiazolo[5,4-d]pyrimidine, thieno[2,3-d]pyrimidine, and thieno[3,2-d]pyrimidine, wherein said 6,7-dihydro-5H-cyclopenta[d]pyrimidine, 5H-pyrrolo[3,2-d]pyrimidine, 7H-pyrrolo[2,3-d]pyrimidine, [1,3]thiazolo[5,4-d]pyrimidine, thieno[2,3-d]pyrimidine, thieno[3,2-d]pyrimidine are optionally substituted on carbon with one or more R², and wherein any —NH— moiety of said 5H-pyrrolo[3,2-d]pyrimidine, and 7H-pyrrolo[2,3-d]pyrimidine is optionally substituted with R²*;

### E is N;

R² is methyl;

### R²* is —S(O)₂R^{2b};

R^{2b} is phenyl, wherein said phenyl is optionally substituted with one or more R²⁰;
R²⁰ is methyl.

In still another aspect, Ring A, together with the pyrimidine to which it is fused, forms a member selected from 7-cyclopropyl-7H-pyrrolo[2,3-d]pyrimidine, 6,7-dihydro-5H-cyclopenta[d]pyrimidine, 1-ethyl-1H-pyrazolo[3,4-d]pyrimidine, 7-methoxyquinazoline, 9-methyl-9H-purine, 6-methyl-7H-pyrrolo[2,3-d]pyrimidine, 7-methylthieno[3,2-d]pyrimidine, 2-(1H-pyrazolo[3,4-d]pyrimidin-1-yl)ethanol, pyrido[2,3-d]pyrimidine, pyrido[3,4-d]pyrimidine, 5H-pyrrolo[3,2-d]pyrimidine, 7H-pyrrolo[2,3-d]pyrimidine, thieno[2,3-d]pyrimidine, and 6-(trifluoromethyl)-7H-pyrrolo[2,3-d]pyrimidine; and

### E is N.

In yet another aspect, Ring A, together with the pyrimidine to which it is fused, forms a member selected from 7-cyclopropyl-7H-pyrrolo[2,3-d]pyrimidine, 1-ethyl-1H-pyrazolo[3,4-d]pyrimidine, 9-methyl-9H-purine, 6-methyl-7H-pyrrolo[2,3-d]pyrimidine, 7-methylthieno[3,2-d]pyrimidine, 2-(1H-pyrazolo[3,4-d]pyrimidin-1-yl)ethanol, 5H-pyrrolo[3,2-d]pyrimidine, 7H-pyrrolo[2,3-d]pyrimidine, thieno[2,3-d]pyrimidine, and 6-(trifluoromethyl)-7H-pyrrolo[2,3-d]pyrimidine; and

### E is N.

In a further aspect, Ring A, together with pyrimidine to which it is fused, forms a member selected from 6,7-dihydro-5H-cyclopenta[d]pyrimidine, 5-[(4-methylphenyl)sulfonyl]-5H-pyrrolo[3,2-d]pyrimidine, 7-[(4-methylphenyl)sulfonyl]-7H-pyrrolo[2,3-d]pyrimidine, 2-methyl[1,3]thiazolo[5,4-d]pyrimidine, 7-methylthieno[3,2-d]pyrimidine, 5H-pyrrolo[3,2-d]pyrimidine, 7H-pyrrolo[2,3-d]pyrimidine, and thieno[2,3-d]pyrimidine; and

### E is N.

In still a further aspect, Ring A, together with the pyrimidine to which it is fused, forms a member selected from 9-methyl-9H-purine and 7H-pyrrolo[2,3-d]pyrimidine; and

### E is N.

### Ring B

In one aspect, Ring B is 6-membered heteroaryl, wherein said 6-membered heteroaryl is optionally substituted with one or more R⁵; and

R⁵ is halo.

In another aspect, Ring B is 6-membered heteroaryl, wherein said 6-membered heteroaryl is substituted with at least one R⁵; and

R⁵ is halo.

In still another aspect, Ring B is selected from pyridinyl and pyrimidinyl, wherein said pyridinyl and pyrimidinyl are optionally substituted with one or more R⁵; and

R⁵ is halo.

In yet another aspect, Ring B is pyrimidinyl, wherein said pyrimidinyl is optionally substituted with one or more R⁵; and

R⁵ is halo.

In a further aspect, Ring B is pyrimidinyl, wherein said pyrimidinyl is substituted with at least one R⁵; and

R⁵ is halo.

In still a further aspect, Ring B is pyrimidin-2-yl, wherein said pyrimidin-2-yl is optionally substituted with one or more R⁵; and

R⁵ is fluoro.

In yet a further aspect, Ring B is pyrimidin-2-yl, wherein said pyrimidin-2-yl is substituted with at least one R⁵; and

R⁵ is fluoro.

In one aspect, Ring B is selected from 3,5-difluoropyridin-2-yl and 5-fluoropyrimidin-2-yl.

In another aspect, Ring B is 5-fluoropyrimidin-2-yl.

### E

In one aspect, E is N.

### R¹*

In one aspect, R¹* is C₁₋₆alkyl.

In another aspect, R¹* is methyl.

### R⁴

In one aspect, R⁴ is C₁₋₆alkyl, wherein said C₁₋₆alkyl is optionally substituted with one or more R⁴⁰;

### R⁴⁰ is —OR^{40a}; and

R^{40a} is C₁₋₆alkyl.

In another aspect, R⁴ is C₁₋₆alkyl.

In still another aspect, R⁴ is selected from methyl and methoxymethyl.

In yet another aspect, R⁴ is methyl.

### Ring A, Ring B, E, R¹*, and R⁴

In one aspect, Ring A is selected from fused 5- or 6-membered heterocycle and fused 5- or 6-membered carbocycle, wherein said fused 5- or 6-membered heterocycle and fused 5- or 6-membered carbocycle are optionally substituted on carbon with one or more R², and wherein any —NH— moiety of said fused 5- or 6-membered heterocycle is optionally substituted with R²*;

Ring B is 6-membered heteroaryl, wherein said 6-membered heteroaryl is optionally substituted with one or more R⁵;

### E is N;

R¹* is C₁₋₆alkyl;
R² in each occurrence is independently selected from halo, C₁₋₆alkyl, 5- or 6-membered heterocyclyl, —OR^{2a}, and —N(R^{2a})₂, wherein said C₁₋₆alkyl is optionally substituted with one or more R²⁰;
R²* in each occurrence is independently selected from C₁₋₆alkyl and 3- to 5-membered carbocyclyl, wherein said C₁₋₆alkyl is optionally substituted with one or more R²⁰;
R^{2a} in each occurrence is independently selected from H, C₁₋₆alkyl, and 3- to 5-membered carbocyclyl;
R⁴ is C₁₋₆alkyl, wherein said C₁₋₆alkyl is optionally substituted with one or more R⁴⁰;
R⁵ is halo;
R²⁰ in each occurrence is independently selected from halo and —OH;

### R⁴⁰ is —OR^{40a}; and

R^{40a} is C₁₋₆alkyl.

In another aspect, Ring A is selected from fused 5-membered carbocycle and fused 5-membered heterocycle, wherein said fused 5-membered carbocycle and fused 5-membered heterocycle are optionally substituted on carbon with one or more R², and wherein any —NH— moiety of said fused 5-membered heterocycle is optionally substituted with R²*;

Ring B is 6-membered heteroaryl, wherein said 6-membered heteroaryl is optionally substituted with one or more R⁵;

### E is N;

R¹* is C₁₋₆alkyl;
R² is C₁₋₆alkyl;

### R²* is —S(O)₂R^{2b};

R^{2b} is phenyl, wherein said phenyl is optionally substituted with one or more R²⁰;
R⁴ is C₁₋₆alkyl;
R⁵ is halo; and
R²⁰ is C₁₋₆alkyl.

In still another aspect, Ring A is selected from fused pyrazole, fused pyridine, fused pyrrole, fused thiazole, and fused thiophene, wherein said fused pyrazole, fused pyridine, fused pyrrole, fused thiazole, and fused thiophene are optionally substituted on carbon with one or more R²; and wherein the —NH— moiety of said fused pyrrole and fused pyrazole is optionally substituted with R²*;

Ring B is selected from pyridinyl and pyrimidinyl, wherein said pyridinyl and pyrimidinyl are optionally substituted with one or more R⁵;

### E is N;

R¹* is methyl;
R² in each occurrence is independently selected from halo, C₁₋₆alkyl, morpholin-4-yl, —OH, and —N(R^{2a})₂, wherein said C₁₋₆alkyl in each occurrence is optionally substituted with halo;
R²* is selected from C₁₋₆alkyl and 3- to 5-membered carbocyclyl, wherein said C₁₋₆alkyl is optionally substituted with one or more R²⁰;
R^{2a} in each occurrence is independently selected from H and 3- to 5-membered carbocyclyl;
R⁴* is C₁₋₆alkyl, wherein said C₁₋₆alkyl is optionally substituted with one or more R⁴⁰;
R⁵ is halo;
R⁴⁰ is C₁₋₆alkyl; and
R²⁰ in each occurrence is independently selected from halo and —OH.

In yet another aspect, Ring A is selected from fused cyclopentane, fused pyrrole, fused thiazole, and fused thiophene, wherein said fused cyclopentane, fused pyrrole, fused thiazole, and fused thiophene are optionally substituted on carbon with one or more R², and wherein the —NH— moiety of said fused pyrrole is optionally substituted with R²*;

Ring B is pyrimidinyl, wherein said pyrimidinyl is optionally substituted with one or more R⁵;

### E is N;

R¹* is C₁₋₆alkyl;
R² is C₁₋₆alkyl;

### R²* is —S(O)₂R^{2b};

R^{2b} is phenyl, wherein said phenyl is optionally substituted with one or more R²⁰;
R⁴ is C₁₋₆alkyl;
R⁵ is halo; and
R²⁰ is C₁₋₆alkyl.

In a further another aspect, Ring A, together with the pyrimidine to which it is fused forms a member selected from 6,7-dihydro-5H-cyclopenta[d]pyrimidine, 5H-pyrrolo[3,2-d]pyrimidine, 7H-pyrrolo[2,3-d]pyrimidine, [1,3]thiazolo[5,4-d]pyrimidine, thieno[2,3-d]pyrimidine, and thieno[3,2-d]pyrimidine, wherein said 6,7-dihydro-5H-cyclopenta[d]pyrimidine, 5H-pyrrolo[3,2-d]pyrimidine, 7H-pyrrolo[2,3-d]pyrimidine, [1,3]thiazolo[5,4-d]pyrimidine, thieno[2,3-d]pyrimidine, thieno[3,2-d]pyrimidine are optionally substituted on carbon with one or more R², and wherein any —NH— moiety of said 5H-pyrrolo[3,2-d]pyrimidine, and 7H-pyrrolo[2,3-d]pyrimidine is optionally substituted with R²*;

Ring B is pyrimidin-2-yl, wherein said pyrimidin-2-yl is optionally substituted with one or more R⁵;

### E is N;

R² is methyl;

### R²* is —S(O)₂R^{2b};

R^{2b} is phenyl, wherein said phenyl is optionally substituted with one or more R²⁰;
R⁴ is methyl;
R⁵ is fluoro;
R²⁰ is methyl.

In still a further aspect, Ring A, together with pyrimidine to which it is fused forms a member selected from 6,7-dihydro-5H-cyclopenta[d]pyrimidine, 5-[(4-methylphenyl)sulfonyl]-5H-pyrrolo[3,2-d]pyrimidine, 7-[(4-methylphenyl)sulfonyl]-7H-pyrrolo[2,3-d]pyrimidine, 2-methyl[1,3]thiazolo[5,4-d]pyrimidine, 7-methylthieno[3,2-d]pyrimidine, 5H-pyrrolo[3,2-d]pyrimidine, 7H-pyrrolo[2,3-d]pyrimidine, and thieno[2,3-d]pyrimidine;

Ring B is 5-fluoropyrimidin-2-yl;

### E is N;

R¹* is methyl; and
R⁴ is methyl.

In yet a further aspect, Ring A, together with the pyrimidine to which it is fused, forms a member selected from 7-cyclopropyl-7H-pyrrolo[2,3-d]pyrimidine, 6,7-dihydro-5H-cyclopenta[d]pyrimidine, 1-ethyl-1H-pyrazolo[3,4-d]pyrimidine, 7-methoxyquinazoline, 9-methyl-9H-purine, 6-methyl-7H-pyrrolo[2,3-d]pyrimidine, 7-methylthieno[3,2-d]pyrimidine, 2-(1H-pyrazolo[3,4-d]pyrimidin-1-yl)ethanol, pyrido[2,3-d]pyrimidine, pyrido[3,4-d]pyrimidine, 5H-pyrrolo[3,2-d]pyrimidine, 7H-pyrrolo[2,3-d]pyrimidine, thieno[2,3-d]pyrimidine, and 6-(trifluoromethyl)-7H-pyrrolo[2,3-d]pyrimidine; and

Ring B is selected from 3,5-difluoropyridin-2-yl and 5-fluoropyrimidin-2-yl;

### E is N;

R¹* is methyl; and
R⁴ is selected from methyl and methoxymethyl.

In one aspect, the compound of Formula (I) is a compound of Formula (Ia):

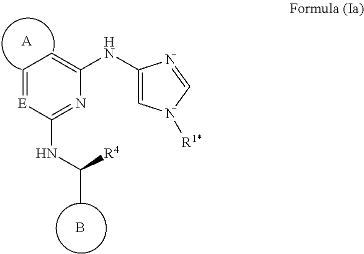

or a pharmaceutically acceptable salt thereof, wherein Ring A, Ring B, E, R¹*, and R⁴ are as defined hereinabove.

In another aspect, the compound of Formula (I) is a compound of Formula (Ia):

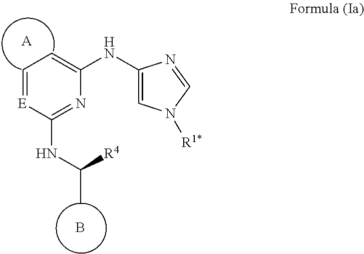

or a pharmaceutically acceptable salt thereof, wherein:
Ring A is selected from fused 5- or 6-membered heterocycle and fused 5- or 6-membered carbocycle, wherein said fused 5- or 6-membered heterocycle and fused 5- or 6-membered carbocycle are optionally substituted on carbon with one or more R², and wherein any —NH— moiety of said fused 5- or 6-membered heterocycle is optionally substituted with R²*;
Ring B is 6-membered heteroaryl, wherein said 6-membered heteroaryl is optionally substituted with one or more R⁵;

### E is N;

R¹* is C₁₋₆alkyl;
R² in each occurrence is independently selected from halo, C₁₋₆alkyl, 5- or 6-membered heterocyclyl, —OR^{2a}, and —N(R^{2a})₂, wherein said C₁₋₆alkyl is optionally substituted with one or more R²⁰;
R²* in each occurrence is independently selected from C₁₋₆alkyl and 3- to 5-membered carbocyclyl, wherein said C₁₋₆alkyl is optionally substituted with one or more R²⁰;
R^{2a} in each occurrence is independently selected from H, C₁₋₆alkyl, and 3- to 5-membered carbocyclyl;
R⁴ is C₁₋₆alkyl, wherein said C₁₋₆alkyl is optionally substituted with one or more R⁴⁰;
R⁵ is halo;
R²⁰ in each occurrence is independently selected from halo and —OH;

### R⁴⁰ is —OR^{40a}; and

R^{40a} is C₁₋₆alkyl.

In one aspect, there is provided a compound selected from:
N²-[(1S)-1-(5-fluoropyrimidin-2-yl)ethyl]-N⁴-(1-methyl-1H-imidazol-4-yl)thieno[2,3-d]pyrimidine-2,4-diamine;
N²-[(1R)-1-(5-fluoropyrimidin-2-yl)ethyl]-N⁴-(1-methyl-1H-imidazol-4-yl)thieno[2,3-d]pyrimidine-2,4-diamine;
N²-[(1S)-1-(5-fluoropyrimidin-2-yl)ethyl]-7-methyl-N⁴-(1-methyl-1H-imidazol-4-yl)thieno[3,2-d]pyrimidine-2,4-diamine;
N²-[(1R)-1-(5-fluoropyrimidin-2-yl)ethyl]-7-methyl-N⁴-(1-methyl-1H-imidazol-4-yl)thieno[3,2-d]pyrimidine-2,4-diamine;
N²-[(1S)-1-(5-fluoropyrimidin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine;
N²-[(1R)-1-(5-fluoropyrimidin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine;
N²-[(1S)-1-(5-fluoropyrimidin-2-yl)ethyl]-N⁴-(1-methyl-1H-imidazol-4-yl)-7H-pyrrolo[2,3-d]pyrimidine-2,4-diamine;
N²-[(1R)-1-(5-fluoropyrimidin-2-yl)ethyl]-N⁴-(1-methyl-1H-imidazol-4-yl)-7H-pyrrolo[2,3-d]pyrimidine-2,4-diamine;
N²-[(1S)-1-(5-fluoropyrimidin-2-yl)ethyl]-N⁴-(1-methyl-1H-imidazol-4-yl)-5-[(4-methylphenyl)sulfonyl]-5H-pyrrolo[3,2-d]pyrimidine-2,4-diamine;
N²-[(1R)-1-(5-fluoropyrimidin-2-yl)ethyl]-N⁴-(1-methyl-1H-imidazol-4-yl)-5-[(4-methylphenyl)sulfonyl]-5H-pyrrolo[3,2-d]pyrimidine-2,4-diamine;
N²-[(1S)-1-(5-fluoropyrimidin-2-yl)ethyl]-N⁴-(1-methyl-1H-imidazol-4-yl)-5H-pyrrolo[3,2-d]pyrimidine-2,4-diamine;
N²-[(1R)-1-(5-fluoropyrimidin-2-yl)ethyl]-N⁴-(1-methyl-1H-imidazol-4-yl)-5H-pyrrolo[3,2-d]pyrimidine-2,4-diamine;
N⁵-[(1S)-1-(5-fluoropyrimidin-2-yl)ethyl]-2-methyl-N⁷-(1-methyl-1H-imidazol-4-yl) [1,3]thiazolo[5,4-d]pyrimidine-5,7-diamine;
N⁵-[(1R)-1-(5-fluoropyrimidin-2-yl)ethyl]-2-methyl-N⁷-(1-methyl-1H-imidazol-4-yl)[1,3]thiazolo[5,4-d]pyrimidine-5,7-diamine;
N²-[(1S)-1-(5-fluoropyrimidin-2-yl)ethyl]-N⁴-(1-methyl-1H-imidazol-4-yl)-6,7-dihydro-5H-cyclopenta[c]pyrimidine-2,4-diamine;
N²-[(1R)-1-(5-fluoropyrimidin-2-yl)ethyl]-N⁴-(1-methyl-1H-imidazol-4-yl)-6,7-dihydro-5H-cyclopenta[c]pyrimidine-2,4-diamine;
1-ethyl-N⁶-[(1S)-1-(5-fluoropyrimidin-2-yl)ethyl]-N⁴-(1-methyl-1H-imidazol-4-yl)-1H-pyrazolo[3,4-d]pyrimidine-4,6-diamine;
1-ethyl-N⁶-[(1R)-1-(5-fluoropyrimidin-2-yl)ethyl]-N⁴-(1-methyl-1H-imidazol-4-yl)-1H-pyrazolo[3,4-d]pyrimidine-4,6-diamine;
N²-[(1S)-1-(5-fluoropyrimidin-2-yl)ethyl]-N⁴-(1-methyl-1H-imidazol-4-yl)pteridine-2,4-diamine;
N²-[(1R)-1-(5-fluoropyrimidin-2-yl)ethyl]-N⁴-(1-methyl-1H-imidazol-4-yl)pteridine-2,4-diamine;
N⁶-[(1S)-1-(5-fluoropyrimidin-2-yl)ethyl]-1-methyl-N⁴-(1-methyl-1H-imidazol-4-yl)-1H-pyrazolo[3,4-d]pyrimidine-4,6-diamine;
N⁶-[(1R)-1-(5-fluoropyrimidin-2-yl)ethyl]-1-methyl-N⁴-(1-methyl-1H-imidazol-4-yl)-1H-pyrazolo[3,4-d]pyrimidine-4,6-diamine;
N²-[(1S)-1-(3,5-difluoropyridin-2-yl)ethyl]-N⁴-(1-methyl-1H-imidazol-4-yl)pyrido[2,3-d]pyrimidine-2,4-diamine;
N²-[(1R)-1-(3,5-difluoropyridin-2-yl)ethyl]-N⁴-(1-methyl-1H-imidazol-4-yl)pyrido[2,3-d]pyrimidine-2,4-diamine;
N⁶-[(1S)-1-(3,5-difluoropyridin-2-yl)-2-methoxyethyl]-1-methyl-N⁴-(1-methyl-1H-imidazol-4-yl)-1H-pyrazolo[3,4-d]pyrimidine-4,6-diamine;
N⁶-[(1R)-1-(3,5-difluoropyridin-2-yl)-2-methoxyethyl]-1-methyl-N⁴-(1-methyl-1H-imidazol-4-yl)-1H-pyrazolo[3,4-d]pyrimidine-4,6-diamine;
N⁶-[(1S)-1-(3,5-difluoropyridin-2-yl)ethyl]-1-methyl-N⁴-(1-methyl-1H-imidazol-4-yl)-1H-pyrazolo[3,4-d]pyrimidine-4,6-diamine;
N⁶-[(1R)-1-(3,5-difluoropyridin-2-yl)ethyl]-1-methyl-N⁴-(1-methyl-1H-imidazol-4-yl)-1H-pyrazolo[3,4-d]pyrimidine-4,6-diamine;
2-(6-{[(1S)-1-(5-fluoropyrimidin-2-yl)ethyl]amino}-4-[(1-methyl-1H-imidazol-4-yl)amino]-1H-pyrazolo[3,4-d]pyrimidin-1-yl)ethanol;
2-(6-{[(1R)-1-(5-fluoropyrimidin-2-yl)ethyl]amino}-4-[(1-methyl-1H-imidazol-4-yl)amino]-1H-pyrazolo[3,4-d]pyrimidin-1-yl)ethanol;
N²-[(1S)-1-(5-fluoropyrimidin-2-yl)ethyl]-N⁴-(1-methyl-1H-imidazol-4-yl)pyrido[2,3-d]pyrimidine-2,4-diamine;
N²-[(1R)-1-(5-fluoropyrimidin-2-yl)ethyl]-N⁴-(1-methyl-1H-imidazol-4-yl)pyrido[2,3-d]pyrimidine-2,4-diamine;
N²-[(1S)-1-(5-fluoropyrimidin-2-yl)ethyl]-N⁴-(1-methyl-1H-imidazol-4-yl)-5,6,7,8-tetrahydropyrido[4,3-d]pyrimidine-2,4-diamine;
N²-[(1R)-1-(5-fluoropyrimidin-2-yl)ethyl]-N⁴-(1-methyl-1H-imidazol-4-yl)-5,6,7,8-tetrahydropyrido[4,3-d]pyrimidine-2,4-diamine;
N²-[(1S)-1-(5-fluoropyrimidin-2-yl)ethyl]-N⁴-(1-methyl-1H-imidazol-4-yl)-6,7-dihydro-5H-pyrrolo[3,4-d]pyrimidine-2,4-diamine;
N²-[(1R)-1-(5-fluoropyrimidin-2-yl)ethyl]-N⁴-(1-methyl-1H-imidazol-4-yl)-6,7-dihydro-5H-pyrrolo[3,4-d]pyrimidine-2,4-diamine;
N²-[(1S)-1-(5-fluoropyrimidin-2-yl)ethyl]-N⁴-(1-methyl-1H-imidazol-4-yl)-6-(trifluoromethyl)-7H-pyrrolo[2,3-d]pyrimidine-2,4-diamine;
N²-[(1R)-1-(5-fluoropyrimidin-2-yl)ethyl]-N⁴-(1-methyl-1H-imidazol-4-yl)-6-(trifluoromethyl)-7H-pyrrolo[2,3-d]pyrimidine-2,4-diamine;
N²-[(1S)-1-(5-Fluoropyrimidin-2-yl)ethyl]-6-methyl-N⁴-(1-methyl-1H-imidazol-4-yl)-7H-pyrrolo[2,3-d]pyrimidine-2,4-diamine;
N²-[(1R)-1-(5-Fluoropyrimidin-2-yl)ethyl]-6-methyl-N⁴-(1-methyl-1H-imidazol-4-yl)-7H-pyrrolo[2,3-d]pyrimidine-2,4-diamine;
N²-[(1S)-1-(5-fluoropyrimidin-2-yl)ethyl]-6-methyl-N⁴-(1-methyl-1H-imidazol-4-yl)-7-[(4-methylphenyl)sulfonyl]-7H-pyrrolo[2,3-d]pyrimidine-2,4-diamine;
N²-[(1R)-1-(5-fluoropyrimidin-2-yl)ethyl]-6-methyl-N⁴-(1-methyl-1H-imidazol-4-yl)-7-[(4-methylphenyl)sulfonyl]-7H-pyrrolo[2,3-d]pyrimidine-2,4-diamine;
7-(2-fluoroethyl)-N²-[(1S)-1-(5-fluoropyrimidin-2-yl)ethyl]-N⁴-(1-methyl-1H-imidazol-4-yl)-7H-pyrrolo[2,3-d]pyrimidine-2,4-diamine;
7-(2-fluoroethyl)-N²-[(1R)-1-(5-fluoropyrimidin-2-yl)ethyl]-N⁴-(1-methyl-1H-imidazol-4-yl)-7H-pyrrolo[2,3-d]pyrimidine-2,4-diamine;
N²-[(1S)-1-(5-fluoropyrimidin-2-yl)ethyl]-7-methyl-N⁴-(1-methyl-1H-imidazol-4-yl)-7H-pyrrolo[2,3-d]pyrimidine-2,4-diamine;
N²-[(1R)-1-(5-fluoropyrimidin-2-yl)ethyl]-7-methyl-N⁴-(1-methyl-1H-imidazol-4-yl)-7H-pyrrolo[2,3-d]pyrimidine-2,4-diamine;
7-cyclopropyl-N²-[(1S)-1-(5-fluoropyrimidin-2-yl)ethyl]-N⁴-(1-methyl-1H-imidazol-4-yl)-7H-pyrrolo[2,3-d]pyrimidine-2,4-diamine;
7-cyclopropyl-N²-[(1R)-1-(5-fluoropyrimidin-2-yl)ethyl]-N⁴-(1-methyl-1H-imidazol-4-yl)-7H-pyrrolo[2,3-d]pyrimidine-2,4-diamine;
N²-[(1S)-1-(3,5-difluoropyridin-2-yl)ethyl]-N⁴-(1-methyl-1H-imidazol-4-yl)-7H-pyrrolo[2,3-d]pyrimidine-2,4-diamine;
N²-[(1R)-1-(3,5-difluoropyridin-2-yl)ethyl]-N⁴-(1-methyl-1H-imidazol-4-yl)-7H-pyrrolo[2,3-d]pyrimidine-2,4-diamine;
N²-[(1S)-1-(3,5-difluoropyridin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine;
N²-[(1R)-1-(3,5-difluoropyridin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine;
N²-[(1S)-1-(5-methoxypyrimidin-2-yl)ethyl]-N⁴-(1-methyl-1H-imidazol-4-yl)-7H-pyrrolo[2,3-d]pyrimidine-2,4-diamine;
N²-[(1R)-1-(5-methoxypyrimidin-2-yl)ethyl]-N⁴-(1-methyl-1H-imidazol-4-yl)-7H-pyrrolo[2,3-d]pyrimidine-2,4-diamine;
N²-[(1S)-1-(5-fluoropyrimidin-2-yl)ethyl]-6-methoxy-N⁴-(1-methyl-1H-imidazol-4-yl)quinazoline-2,4-diamine;
N²-[(1R)-1-(5-fluoropyrimidin-2-yl)ethyl]-6-methoxy-N⁴-(1-methyl-1H-imidazol-4-yl)quinazoline-2,4-diamine;
N²-[(1S)-1-(3,5-difluoropyridin-2-yl)ethyl]-6-methoxy-N⁴-(1-methyl-1H-imidazol-4-yl)quinazoline-2,4-diamine;
N²-[(1R)-1-(3,5-difluoropyridin-2-yl)ethyl]-6-methoxy-N⁴-(1-methyl-1H-imidazol-4-yl)quinazoline-2,4-diamine;
N²-[(1S)-1-(5-fluoropyrimidin-2-yl)ethyl]-7-methoxy-N⁴-(1-methyl-1H-imidazol-4-yl)quinazoline-2,4-diamine;
N²-[(1R)-1-(5-fluoropyrimidin-2-yl)ethyl]-7-methoxy-N⁴-(1-methyl-1H-imidazol-4-yl)quinazoline-2,4-diamine;
N²-[(1S)-1-(3,5-difluoropyridin-2-yl)ethyl]-6-fluoro-N⁴-(1-methyl-1H-imidazol-4-yl)pyrido[2,3-d]pyrimidine-2,4-diamine;
N²-[(1R)-1-(3,5-difluoropyridin-2-yl)ethyl]-6-fluoro-N⁴-(1-methyl-1H-imidazol-4-yl)pyrido[2,3-d]pyrimidine-2,4-diamine;
N²-[(1S)-1-(5-fluoropyrimidin-2-yl)ethyl]-N⁴-(1-methyl-1H-imidazol-4-yl)-7-(trifluoromethyl)pyrido[2,3-d]pyrimidine-2,4-diamine;
N²-[(1R)-1-(5-fluoropyrimidin-2-yl)ethyl]-N⁴-(1-methyl-1H-imidazol-4-yl)-7-(trifluoromethyl)pyrido[2,3-d]pyrimidine-2,4-diamine;
N²-[(1S)-1-(3,5-difluoropyridin-2-yl)ethyl]-N⁴-(1-methyl-1H-imidazol-4-yl)-7-(trifluoromethyl)pyrido[2,3-d]pyrimidine-2,4-diamine;
N²-[(1R)-1-(3,5-difluoropyridin-2-yl)ethyl]-N⁴-(1-methyl-1H-imidazol-4-yl)-7-(trifluoromethyl)pyrido[2,3-d]pyrimidine-2,4-diamine;
2-{[(1S)-1-(5-fluoropyrimidin-2-yl)ethyl]amino}-4-[(1-methyl-1H-imidazol-4-yl)amino]pyrido[2,3-d]pyrimidin-7-ol;
2-{[(1R)-1-(5-fluoropyrimidin-2-yl)ethyl]amino}-4-[(1-methyl-1H-imidazol-4-yl)amino]pyrido[2,3-d]pyrimidin-7-ol;
2-{[(1S)-1-(3,5-difluoropyridin-2-yl)ethyl]amino}-4-[(1-methyl-1H-imidazol-4-yl)amino]pyrido[2,3-d]pyrimidin-7-ol;
2-{[(1R)-1-(3,5-difluoropyridin-2-yl)ethyl]amino}-4-[(1-methyl-1H-imidazol-4-yl)amino]pyrido[2,3-d]pyrimidin-7-ol;
N⁷-cyclopropyl-N²-[(1S)-1-(5-fluoropyrimidin-2-yl)ethyl]-N⁴-(1-methyl-1H-imidazol-4-yl)pyrido[2,3-d]pyrimidine-2,4,7-triamine;
N⁷-cyclopropyl-N²-[(1R)-1-(5-fluoropyrimidin-2-yl)ethyl]-N⁴-(1-methyl-1H-imidazol-4-yl)pyrido[2,3-d]pyrimidine-2,4,7-triamine;
N²-[(1S)-1-(5-fluoropyrimidin-2-yl)ethyl]-N⁴-(1-methyl-1H-imidazol-4-yl)-7-morpholin-4-ylpyrido[2,3-d]pyrimidine-2,4-diamine;
N²-[(1R)-1-(5-fluoropyrimidin-2-yl)ethyl]-N⁴-(1-methyl-1H-imidazol-4-yl)-7-morpholin-4-ylpyrido[2,3-d]pyrimidine-2,4-diamine;
6-fluoro-N²-[(1S)-1-(5-fluoropyrimidin-2-yl)ethyl]-N⁴-(1-methyl-1H-imidazol-4-yl)pyrido[2,3-d]pyrimidine-2,4-diamine;
6-fluoro-N²-[(1R)-1-(5-fluoropyrimidin-2-yl)ethyl]-N⁴-(1-methyl-1H-imidazol-4-yl)pyrido[2,3-d]pyrimidine-2,4-diamine;
N²,N⁷-bis[(1S)-1-(5-fluoropyrimidin-2-yl)ethyl]-N⁴-(1-methyl-1H-imidazol-4-yl)pyrido[2,3-d]pyrimidine-2,4,7-triamine;
N²,N⁷-bis[(1R)-1-(5-fluoropyrimidin-2-yl)ethyl]-N⁴-(1-methyl-1H-imidazol-4-yl)pyrido[2,3-d]pyrimidine-2,4,7-triamine;
N²-[(1S)-1-(3,5-difluoropyridin-2-yl)ethyl]-N⁴-(1-methyl-1H-imidazol-4-yl)-7-morpholin-4-ylpyrido[2,3-d]pyrimidine-2,4-diamine;
N²-[(1R)-1-(3,5-difluoropyridin-2-yl)ethyl]-N⁴-(1-methyl-1H-imidazol-4-yl)-7-morpholin-4-ylpyrido[2,3-d]pyrimidine-2,4-diamine;
N²-[(1S)-1-(5-fluoropyrimidin-2-yl)ethyl]-N⁴-(1-methyl-1H-imidazol-4-yl)pyrido[3,4-d]pyrimidine-2,4-diamine;
N²-[(1R)-1-(5-fluoropyrimidin-2-yl)ethyl]-N⁴-(1-methyl-1H-imidazol-4-yl)pyrido[3,4-d]pyrimidine-2,4-diamine;
7-chloro-N²-[(1S)-1-(5-fluoropyrimidin-2-yl)ethyl]-N⁴-(1-methyl-1H-imidazol-4-yl)pyrido[2,3-d]pyrimidine-2,4-diamine;
7-chloro-N²-[(1R)-1-(5-fluoropyrimidin-2-yl)ethyl]-N⁴-(1-methyl-1H-imidazol-4-yl)pyrido[2,3-d]pyrimidine-2,4-diamine;
N²-[(1S)-1-(3,5-difluoropyridin-2-yl)ethyl]-N⁴-(1-methyl-1H-imidazol-4-yl)pyrido[3,4-d]pyrimidine-2,4-diamine;
N²-[(1R)-1-(3,5-difluoropyridin-2-yl)ethyl]-N⁴-(1-methyl-1H-imidazol-4-yl)pyrido[3,4-d]pyrimidine-2,4-diamine;
7-chloro-N²-[(1S)-1-(3,5-difluoropyridin-2-yl)ethyl]-N⁴-(1-methyl-1H-imidazol-4-yl)pyrido[2,3-d]pyrimidine-2,4-diamine;
7-chloro-N²-[(1R)-1-(3,5-difluoropyridin-2-yl)ethyl]-N⁴-(1-methyl-1H-imidazol-4-yl)pyrido[2,3-d]pyrimidine-2,4-diamine;
N²-[(1S)-1-(5-fluoropyrimidin-2-yl)ethyl]-N⁴-(1-methyl-1H-imidazol-4-yl)quinazoline-2,4-diamine;
N²-[(1R)-1-(5-fluoropyrimidin-2-yl)ethyl]-N⁴-(1-methyl-1H-imidazol-4-yl)quinazoline-2,4-diamine;
N⁶-[(1S)-1-(5-fluoropyrimidin-2-yl)ethyl]-N⁴-(1-methyl-1H-imidazol-4-yl)-1H-pyrazolo[3,4-d]pyrimidine-4,6-diamine; and
N⁶-[(1R)-1-(5-fluoropyrimidin-2-yl)ethyl]-N⁴-(1-methyl-1H-imidazol-4-yl)-1H-pyrazolo[3,4-d]pyrimidine-4,6-diamine,
   or a pharmaceutically acceptable salt thereof.

### Utility

The compounds of Formula (I) have utility for the inhibition of the JAK tyrosein kinases, particularly the JAK2 family. The compounds of Formula (I) additionally have utility for the treatment of myeloproliferative disorders, myelodysplastic syndrome and cancer. Methods of treatment target tyrosine kinase activity, particularly the JAK family activity and more particularly JAK2 activity, which is involved in a variety of myeloproliferative disorders, myelodysplastic syndrome and cancer related processes. Inhibitors of tyrosine kinase, particularly the JAK family and more particularly JAK2, are expected to be active against myeloproliferative disorders such as chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and neoplastic disease such as carcinoma of the breast, ovary, lung, colon, prostate or other tissues, as well as leukemias, myelomas and lymphomas, tumors of the central and peripheral nervous system, and other tumor types such as melanoma, fibrosarcoma and osteosarcoma. Tyrosine kinase inhibitors, particularly the JAK family inhibitors and more particularly JAK2 inhibitors are also expected to be useful for the treatment other proliferative diseases including but not limited to autoimmune, inflammatory, neurological, and cardiovascular diseases.

The compounds of Formula (I) have been shown to inhibit tyrosine kinases, particularly the JAK family and more particularly JAK2, as determined by the JAK2 assays (methods 1 to 3) described below.

The compounds of Formula (I) should also be useful as standards and reagents in determining the ability of a potential pharmaceutical to inhibit tyrosine kinases, particularly the JAK family and more particularly JAK2. These would be provided in commercial kits comprising a compound of this invention.

Although the pharmacological properties of the compounds of the Formula (I) may vary with structural change, typical compounds of the Formula (I) are generally believed to possess JAK inhibitory activity at IC₅₀ concentrations (concentrations to achieve 50% inhibition) or doses at a level below 10 μM.

### Method 1

JAK2 kinase activity may be determined by measuring the kinase's ability to phosphorylate synthetic tyrosine residues within a generic polypeptide substrate using an Amplified Luminescent Proximity Assay (Alphascreen) technology (PerkinElmer, 549 Albany Street, Boston, Mass.).

To measure JAK2 kinase activity, a commercially available purified enzyme may be used. The enzyme may be C-terminal His6-tagged, recombinant, human JAK2, amino acids 808-end, (Genbank Accession number NM 004972) expressed by baculovirus in Sf21 cells (Upstate Biotechnology MA). After incubation of the kinase with a biotinylated substrate and adenosine triphosphate (ATP) for 60 minutes at room temperature, the kinase reaction may be stopped by the addition of 30 mM ethylenediaminetetraacetic acid (EDTA). The reaction may be performed in 384 well microtitre plates and the reaction products may be detected with the addition of streptavidin coated Donor Beads and phosphotyrosine-specific antibodies coated Acceptor Beads using the EnVision Multilabel Plate Reader after an overnight incubation at room temperature. “Tween 20” is a registered trademark of ICI Americas, Inc.

### JAK2 Hu Phos AScrn CRIC₅₀ ENZ 5PT JAK2 AS1 JAK2 Mean IC₅₀ (μM) Assay

| | |
|---|---|
| Peptide | TYK2 (Tyr 1054/1055 biotinylated peptide) Cell |
| substrate | signaling Technology #2200B. 402 μM stock. |
| ATP Km | 30 μM |
| Assay | 150 pM JAK2 enzyme, 5 mM ATP, 80 nM Tyk2, |
| conditions | 10 mM MgCl₂, 50 mM Hepes buffer pH |
| | 7.5, 1 mM DTT, 0.025% Tween20. |
| Incubation | 60 minutes, room temperature |
| Termination/ | 6.3 mM HEPES, 30 mM EDTA, 525 μg/ml BSA, |
| Detection | 40 mM NaCl, 0.007% Triton ® X-100, 12 ng/ml |
| conditions | of Donor Beads, 12 ng/ml of Acceptor Beads |
| Detection | overnight, room temperature |
| incubation | |
| Fluometer | Excitation = 680 nm Emission = 570 nm |
| settings | Excitation Time = 180 ms Total Measurement |
| | Time = 550 ms |

### Method 2

Janus kinase 2 (JAK2) activity was also determined by measuring the kinase's ability to phosphorylate a tyrosine residue within a peptide substrate using a mobility shift assay on a Caliper LC3000 reader (Caliper, Hopkinton, Mass.), which measures fluorescence of the phosphorylated and unphosphorylated substrate and calculates a ratiometric value to determine percent turnover.

To measure JAK2 kinase activity, an in-house purified enzyme was used. The enzyme was N-terminal GST-tagged, recombinant, human JAK2 (amino acids 831-1132, PLAZA database pAZB0359) expressed in insect cells. After incubation of the kinase with a FAM labeled SRCtide substrate, adenosine triphosphate (ATP), and MgCl₂ for 90 minutes at room temperature, the kinase reaction was stopped by the addition of 36 mM ethylenediaminetetraacetic acid (EDTA). The reaction was performed in 384 well microtitre plates and the reaction products were detected using the Caliper LC3000 Reader.

| | |
|---|---|
| Peptide | SRCtide (5FAM-GEEPLYWSFPAKKK-NH2) |
| substrate | (Anaspec, San Jose, CA) |
| ATP Km | 10 μM |
| Assay | 0.3 nM JAK2 enzyme, 5 mM ATP, 1.5 μM SRCtide, |
| conditions | 10 mM MgCl₂, 50 mM HEPES buffer |
| | (pH 7.3), 1 mM DTT, 0.01% Tween |
| | 20, 50 μg/ml BSA |
| Incubation | 90 minutes, room temperature |
| Termination/ | 65 mM HEPES, 36 mM EDTA, 0.2% Coatin |
| Detection | Reagent 3 (Caliper, Hopkinton, MA), 0.003% |
| conditions | Tween 20 |
| Caliper | −1.7 PSI, −2000 V downstream voltage, −400 V |
| LC3000 | upstream voltage, 0.2 second sample sip time, |
| settings | 45 second post sip time, 10% laser strength. |

### Method 3

Activity of purified C-terminal His6-tagged human JAK2 kinase was determined in-vitro using an Amplified Luminescent Proximity Homogeneous Assay (ALPHA) (Perkin Elmer, MA), which measures phosphorylation of a biotinylated Tyk (Tyr104/1055) substrate (Cell Signaling Technology, MA, Cat #2200B). Commercially available JAK2 (amino acids 808-end, Genbank Accession number NM 004972, Upstate Biotechnology, MA, Catalog 14-640) was expressed by baculovirus in Sf21 cells and affinity purified by Ni⁺²/NTA agarose.

The phosphorylation of Tyk substrate in the presence and absence of the compound of interest was determined. Briefly, 5 μl of Enzyme/Substrate/adenosine triphosphate (ATP) mix consisting of 1.44 nM JAK2, 192 nM Tyk, and 12 mM ATP in 1.2× buffer was preincubated with 2 μl of compound for 20 minutes at 25° C. Reactions were initiated with 5 ul of Metal mix consisting of 24 mM MgCl₂ in 1.2× buffer and incubated at 25° C. for 90 minutes and reactions were stopped by addition of 5 ul of Detection mix consisting of 20 mM HEPES, 102 mM ethylenediamine tetraacetic acid, 1.65 mg/ml BSA, 136 mM NaCl, 40 μg/ml Streptavidin donor beads (Perkin Elmer, MA, Catalog #6760002), and 40 ug/ml phosphotyrosine-specific antibody coated acceptor beads (Perkin Elmer, MA, Catalog #6760620). Plates were incubated at 25° C. for 18 hours in the dark. Phosphorylated substrate was detected by an EnVision plate reader (Perkin Elmer, MA) 680 nm excitation, 520-620 nm emission. Data was graphed and IC₅₀s calculated using Excel Fit (Microsoft).

Although the pharmacological properties of the compounds of the Formula (I) may vary with structural change, typical compounds of the Formula (I) are generally believed to possess JAK inhibitory activity at IC₅₀ concentrations (concentrations to achieve 50% inhibition) or doses at a level below 10 μM.

When tested in assays based on the in-vitro assays (methods 1-3) described above, the JAK inhibitory activities of the following examples were measured at the IC₅₀s (μM) shown in Table 1. A hyphen indicates that an IC₅₀ measurement is not provided for that particular compound, and is not meant to imply that the particular compound does not possess IC₅₀ activity.

| | Assay | Assay | Assay |
|---|---|---|---|
| Example | (Method 1) | (Method 2) | Method (3) |
| 1 | — | — | — |
| 1(a) | 28.8 | — | — |
| 1(b) | <0.003 | — | — |
| 2 | — | — | — |
| 2(a) | 9.3 | — | — |
| 2(b) | 0.004 | — | — |
| 3 | 0.213 | — | — |
| 4 | — | — | — |
| 4(a) | 1.9 | — | — |
| 4(b) | <0.003 | — | — |
| 5 | — | — | — |
| 6 | <0.003 | — | — |
| 7 | 1.9 | — | — |
| 7(a) | 0.57 | — | — |
| 7(b) | 21.6 | — | — |
| 8 | 0.016 | — | — |
| 8(a) | 20.03 | — | — |
| 8(b) | <0.003 | — | — |
| 9 | — | — | — |
| 9(a) | — | — | 13.7 |
| 9(b) | — | 0.04 | — |
| 10 | — | — | — |
| 10(a) | 6.4 | — | — |
| 10(b) | 0.20 | — | — |
| 11 | — | — | — |
| 11(a) | 6.4 | — | — |
| 11(b) | <0.003 | — | — |
| 12 | — | — | — |
| 12(a) | <0.003 | — | — |
| 12(b) | 0.47 | — | — |
| 13 | — | — | — |
| 13(a) | 3.24 | — | — |
| 13(b) | <0.003 | — | — |
| 14 | — | — | — |
| 14(a) | 5.4 | — | — |
| 14(b) | <0.003 | — | — |
| 15 | 0.003 | — | — |
| 16 | <0.003 | — | — |
| 17 | — | — | — |
| 17(a) | 0.22 | — | — |
| 17(b) | 2 | — | — |
| 18 | — | — | — |
| 18(a) | 4.3 | — | — |
| 18(b) | 0.45 | — | — |
| 19 | — | 0.013 | — |
| 20 | — | 0.013 | — |
| 21 | — | — | — |
| 22 | — | — | — |
| 22(a) | — | 2.32 | — |
| 22(b) | — | 0.007 | — |
| 23 | — | — | — |
| 23(a) | <0.003 | — | — |
| 23(b) | — | — | — |
| 24 | — | — | — |
| 24(a) | — | — | — |
| 24(b) | — | 0.027 | — |
| 25 | — | — | — |
| 25(a) | — | — | 2.9 |
| 25(b) | — | 0.010 | — |
| 26 | — | — | — |
| 27 | — | 0.27 | — |
| 27(a) | — | 17.6 | — |
| 27(b) | — | 0.52 | — |
| 28 | 0.67 | — | — |
| 29 | 0.50 | — | — |
| 30 | 0.004 | — | — |
| 30(a) | — | — | — |
| 30(b) | — | — | — |
| 31 | — | — | — |
| 31(a) | — | 0.08 | — |
| 31(b) | — | 15 | — |
| 32 | — | 0.035 | — |
| 33 | — | 0.026 | — |
| 34 | — | 0.047 | — |
| 35 | 0.003 | — | — |
| 36 | 0.003 | — | — |
| 37 | — | — | — |
| 38 | — | — | — |
| 38(a) | 0.10 | — | — |
| 38(b) | 0.49 | — | — |
| 39 | 0.003 | — | — |
| 40 | — | — | — |
| 40(a) | 0.49 | — | — |
| 40(b) | <0.003 | — | — |
| 41 | — | — | — |
| 41(a) | <0.003 | — | — |
| 41(b) | 2.74 | — | — |
| 42 | <0.003 | — | — |
| 43 | — | — | — |
| 43(a) | <0.003 | — | — |
| 43(b) | 1.18 | — | — |
| 44 | 0.003 | — | — |
| 45 | <0.003 | — | — |
| 45(a) | 0.064 | — | — |
| 45(b) | 0.003 | — | — |
| 46 | 0.021 | — | — |

In one aspect, there is provided a compound of Formula (I), or a pharmaceutically acceptable salt thereof, for use as a medicament.

In another aspect, there is provided the use of a compound of Formula (I), or a pharmaceutically acceptable salt thereof, in the manufacture of a medicament for the treatment or prophylaxis of myeloproliferative disorders, myelodysplastic syndrome, and cancer, in a warm-blooded animal such as man.

In still another aspect, there is provided the use of a compound of Formula (I), or a pharmaceutically acceptable salt thereof, in the manufacture of a medicament for the treatment or prophylaxis of myeloproliferative disorders, myelodysplastic syndrome and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man.

In yet another aspect, there is provided the use of a compound of Formula (I), or a pharmaceutically acceptable salt thereof, in the manufacture of a medicament for treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man.

In a further aspect, there is provided the use of a compound of Formula (I), or a pharmaceutically acceptable salt thereof, in the manufacture of a medicament for the production of an anti-proliferative effect, in a warm-blooded animal such as man.

In still a further aspect, there is provided the use of a compound of Formula (I), or a pharmaceutically acceptable salt thereof, in the manufacture of a medicament for the production of a JAK inhibitory effect.

In yet a further aspect, there is provided the use of a compound of Formula (I), or a pharmaceutically acceptable salt thereof, in the manufacture of a medicament for the treatment of cancer.

In one aspect, there is provided a method for treating myeloproliferative disorders, myelodysplastic syndrome, and cancer, in a warm-blooded animal such as man, said method comprising administering to said animal an effective amount of a compound of Formula (I), or a pharmaceutically acceptable salt thereof.

In another aspect, there is provided a method for treating myeloproliferative disorders, myelodysplastic syndrome, and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man, said method comprising administering to said animal an effective amount of a compound of Formula (I), or a pharmaceutically acceptable salt thereof.

In still another aspect, there is provided a method for treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man, said method comprising administering to said animal an effective amount of compound of Formula (I), or a pharmaceutically acceptable salt thereof.

In yet another aspect, there is provided a method for producing an anti-proliferative effect in a warm-blooded animal such as man, said method comprising administering to said animal an effective amount of a compound of Formula (I), or a pharmaceutically acceptable salt thereof.

In a further aspect, there is provided a method for producing a JAK inhibitory effect in a warm-blooded animal such as man, said method comprising administering to said animal an effective amount of a compound of Formula (I), or a pharmaceutically acceptable salt thereof.

In still a further aspect, there is provided a method for treating cancer in a warm-blooded animal such as man, said method comprising administering to said animal an effective amount of a compound of Formula (I), or a pharmaceutically acceptable salt thereof.

In yet a further aspect, there is provided a compound of Formula (I), or a pharmaceutically acceptable salt thereof, for use in treating myeloproliferative disorders, myelodysplastic syndrome, and cancer, in a warm-blooded animal such as man.

In one aspect, there is provided a compound of Formula (I), or a pharmaceutically acceptable salt thereof, for use in treating myeloproliferative disorders, myelodysplastic syndrome, and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man.

In another aspect, there is provided a compound of Formula (I), or a pharmaceutically acceptable salt thereof, for use in the treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man.

In still another aspect, there is provided a compound of Formula (I), or a pharmaceutically acceptable salt thereof, for use in the production of an anti-proliferative effect, in a warm-blooded animal such as man.

In yet another further aspect, there is provided a compound of Formula (I), or a pharmaceutically acceptable salt thereof, for use in the production of a JAK inhibitory effect in a warm-blooded animal such as man.

In a further aspect, there is provided a compound of Formula (I), or a pharmaceutically acceptable salt thereof, for use in the treatment of cancer in a warm-blooded animal such as man.

In still a further aspect, where reference is made to the treatment (or prophylaxis) of cancer, it may particularly refer to the treatment (or prophylaxis) of mesoblastic nephroma, mesothelioma, acute myeloblastic leukemia, acute lymphocytic leukemia, multiple myeloma, oesophageal cancer, myeloma, hepatocellular, pancreatic, cervical cancer, Ewings sarcoma, neuroblastoma, Kaposi's sarcoma, ovarian cancer, breast cancer including secretory breast cancer, colorectal cancer, prostate cancer including hormone refractory prostate cancer, bladder cancer, melanoma, lung cancer—non small cell lung cancer (NSCLC), and small cell lung cancer (SCLC), gastric cancer, head and neck cancer, renal cancer, lymphoma, thyroid cancer including papillary thyroid cancer, mesothelioma, leukaemia, tumors of the central and peripheral nervous system, melanoma, fibrosarcoma including congenital fibrosarcoma and osteosarcoma. More particularly it refers to prostate cancer. In addition, more particularly it refers to SCLC, NSCLC, colorectal cancer, ovarian cancer and/or breast cancer. In a further aspect it may refer to hormone refractory prostate cancer.

In yet a further aspect, there is provided a pharmaceutical composition comprising a compound of Formula (I), or a pharmaceutically acceptable salt thereof, and at least one pharmaceutically acceptable carrier, diluent, or excipient.

In one aspect, there is provided a pharmaceutical composition comprising a compound of Formula (I), or a pharmaceutically acceptable salt thereof, and at least one pharmaceutically acceptable carrier, diluent, or excipient.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate; anti-oxidants such as ascorbic acid); coloring agents; flavoring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 4 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

As stated above the size of the dose required for the therapeutic or prophylactic treatment of a particular disease state will necessarily be varied depending on the host treated, the route of administration and the severity of the illness being treated. A daily dose in the range of 0.1-50 mg/kg may be employed. Accordingly, the optimum dosage may be determined by the practitioner who is treating any particular patient.

The anti-cancer treatment defined herein may be applied as a sole therapy or may involve, in addition to the compound of the invention, conventional surgery or radiotherapy or chemotherapy. Such chemotherapy may include one or more of the following categories of anti-tumor agents:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines including 5-fluorouracil and tegafur, raltitrexed, methotrexate, cytosine arabinoside and hydroxyurea); antitumor antibiotics (for example anthracyclines such as adriamycin, bleomycin, doxorubicin, daunomycin, epirubicin, idarubicin, mitomycin-C, dactinomycin and mithramycin); antimitotic agents (for example vinca alkaloids such as vincristine, vinblastine, vindesine and vinorelbine and taxoids such as taxol and taxotere); and topoisomerase inhibitors (for example epipodophyllotoxins such as etoposide and teniposide, amsacrine, topotecan and camptothecin); and proteosome inhibitors (for example bortezomib [Velcade®]); and the agent anegrilide [Agrylin®]; and the agent alpha-interferon;
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors such as marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis (2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family, for example inhibitors or phosphotidylinositol 3-kinase (PI3K) and for example inhibitors of mitogen activated protein kinase (MEK1/2) and for example inhibitors of protein kinase B (PKB/Akt), for example inhibitors of Src tyrosine kinase family and/or Abelson (Abl) tyrosine kinase family such as AZD0530 and dasatinib (BMS-354825) and imatinib mesylate (Gleevec™); and any agents that modify STAT signaling;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
   (vi) vascular damaging agents such as Combretastatin A4 and compounds disclosed in International Patent Applications WO 99/02166, WO 00/40529, WO 00/41669, WO 01/92224, WO 02/04434 and WO 02/08213;
   (vii) antisense therapies, for example those which are directed to the targets listed above, such as ISIS 2503, an anti-ras antisense;
   (viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
   (ix) immunotherapy approaches, including for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies and approaches using the immunomodulatory drugs thalidomide and lenalidomide [Revlimid®]; and
   (x) other treatment regimes including: dexamethasone, proteasome inhibitors (including bortezomib), isotretinoin (13-cis retinoic acid), thalidomide, revemid, Rituxamab, ALIMTA, Cephalon's kinase inhibitors CEP-701 and CEP-2563, anti-Trk or anti-NGF monoclonal antibodies, targeted radiation therapy with 131I-metaiodobenzylguanidine (131I-MIBG), anti-G(D2) monoclonal antibody therapy with or without granulocyte-macrophage colony-stimulating factor (GM-CSF) following chemotherapy.

Such conjoint treatment may be achieved by way of the simultaneous, sequential or separate dosing of the individual components of the treatment. Such combination products employ the compounds of this invention, or pharmaceutically acceptable salts thereof, within the dosage range described hereinbefore and the other pharmaceutically-active agent within its approved dosage range.

In addition to its use in therapeutic medicine, compounds of Formula (I) and pharmaceutically acceptable salts thereof are also useful as pharmacological tools in the development and standardization of in vitro and in vivo test systems for the evaluation of the effects of inhibitors of JAK2 in laboratory animals such as cats, dogs, rabbits, monkeys, rats and mice, as part of the search for new therapeutic agents.

In any of the above-mentioned pharmaceutical composition, process, method, use, medicament, and manufacturing features of the instant invention, any of the alternate embodiments of the compounds of the invention described herein also apply.

In one aspect, the inhibition of JAK activity particularly refers to the inhibition of JAK2 activity.

### Process

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 5^{th} Edition, by Jerry March and Michael Smith, published by John Wiley & Sons 2001, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991) and as described hereinabove.

Compounds of Formula (I) may be prepared in a variety of ways. The Process shown below illustrate some methods for synthesizing compounds of Formula (I) and intermediates which may be used for the synthesis of compounds of Formula (I) (wherein Ring A, Ring B, E, R¹*, and R⁴, unless otherwise defined, are as defined hereinabove). Where a particular solvent or reagent is shown in a Process or referred to in the accompanying text, it is to be understood that the chemist of ordinary skill in the art will be able to modify that solvent or reagent as necessary. The Process is not intended to present an exhaustive list of methods for preparing the compounds of Formula (I); rather, additional techniques of which the skilled chemist is aware may be also be used for the compounds' synthesis. The claims are not intended to be limited to the structures shown in the Process.

The skilled chemist will be able to use and adapt the information contained and referenced within the above references, and accompanying Examples therein and also the Examples herein, to obtain necessary starting materials and products.

In one aspect, compounds of Formula (I), or pharmaceutically acceptable salts thereof, may be prepared by:

1) Process A—reacting a compound of Formula (A):

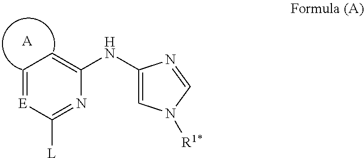

with a compound of Formula (B):

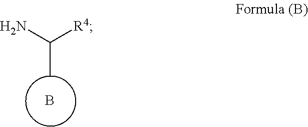

and thereafter if necessary:
i) converting a compound of Formula (I) into another compound of Formula (I);
ii) removing any protecting groups; and/or
iii) forming a pharmaceutically acceptable salt,
   wherein L is a leaving group as described hereinabove.

It is to be understood that protecting groups may be used as necessary. Leaving groups suitable for use in Process A include halo groups such as chloro.

Process A—Compounds of Formula (A) and compounds of Formula (B) may be reacted together in the presence of a suitable solvent, examples of which include ketones such as acetone, alcohols such as ethanol and butanol, and aromatic hydrocarbons such as toluene and N-methylpyrrolid-2-one. The reaction may advantageously occur in the presence of a suitable base, examples of which include inorganic bases such as potassium carbonate and cesium carbonate, and organic bases such as potassium tert-butoxide and sodium tert-butoxide. The reaction may be advantageously performed at a temperature in a range from 0° C. to reflux. Heating the reaction may be particularly advantageous.

In another aspect, compounds of Formula (A) and compounds of Formula (B) may be reacted together under standard Buchwald conditions (for example see J. Am. Chem. Soc., 118, 7215; J. Am. Chem. Soc., 119, 8451; J. Org. Chem., 62, 1568 and 6066), with a suitable base. Examples of suitable bases include inorganic bases such as cesium carbonate, and organic bases such as potassium t-butoxide. Such a reaction may advantageously occur in the presence of a palladium catalyst such as palladium acetate. Examples of solvents suitable for such a reaction include toluene, benzene, dioxane, and xylene.

### EXAMPLES

The invention will now be further described with reference to the following illustrative Examples in which, unless stated otherwise:
(i) temperatures are given in degrees Celsius (° C.); operations are carried out at room temperature or ambient temperature, that is, in a range of 18-25° C.;
(ii) organic solutions were dried over anhydrous magnesium sulfate unless other wise stated; evaporation of organic solvent was carried out using a rotary evaporator under reduced pressure (4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC or liquid chromatography/mass spectroscopy and reaction times are given for illustration only;
(v) final products have satisfactory proton nuclear magnetic resonance (NMR) spectra and/or mass spectra data;
(vi) yields are given for illustration only and are not necessarily those which can be obtained by diligent process development; preparations were repeated if more material was required;
(vii) when given, NMR data is in the form of delta values for major diagnostic protons, given in part per million (ppm) relative to tetramethylsilane (TMS) as an internal standard, determined at 300 MHz in DMSO-d₆ unless otherwise stated;
(viii) chemical symbols have their usual meanings;
(ix) solvent ratio was given in volume:volume (v/v) terms;
(x) “ISCO” refers to normal phase flash column chromatography using pre-packed silica gel cartridges (12 g, 40 g etc.), used according to the manufacturer's instructions, obtained from ISCO, Inc, 4700 Superior Street Lincoln, Nebr., USA;
(xi) “Gilson® chromatography” refers to chromatography using a YMC-AQC 18 reversed phase HPLC Column (unless otherwise indicated) with dimension 20 mm/100 and 50 mm/250 in H₂O/MeCN with 0.1% TFA as mobile phase (unless otherwise stated), used according to the manufacturer's instructions, obtained from Gilson®, Inc. 3000 Parmenter Street, Middleton, Wis. 53562-0027, U.S.A;
(xii) “Biotage®” refers to normal phase flash column chromatography using pre-packed silica gel cartridges (12 g, 40 g, 80 g etc.), used according to the manufacturer's instructions, obtained from Biotage® Inc, 1725 Discovery Drive Charlotteville, Va. 22911, USA;
(xiii) “SFC (super critical fluid chromatography)” refers to Analytical SFC (ASC-1000 Analytical SFC System with a diode array detector) and/or Preparative SFC (APS-1000 AutoPrep Preparative SFC), used according to the manufacturer's instruction, obtained from SFC Mettler Toledo AutoChem, Inc. 7075 Samuel Morse Drive Columbia Md. 21046, USA.;
(xiv) Chiralcel OJ® and Chiralcel AD-H®, Chiralcel AD-S® or Chiralpak® columns are used according to the manufacturer's instruction, and are obtained from Chiral Technologies, Inc. 800 North Five Points Road West Chester, Pa. 19380, USA;
(xv) Parr Hydrogenator or Parr shaker type hydrogenators are systems for treating chemicals with hydrogen in the presence of a catalyst at pressures up to 5 atmospheres (60 psi) and temperatures to 80° C.;
(xvi) the following abbreviations may have been used:
   BINAP 2,2′-bis(diphenylphosphino)-1,1′-binapthyl
   Boc₂O tert-butyloxycarbonyl anhydride
   DAST Diethylaminosulfur trifluoride
   DCM dichloromethane
   DIPEA N,N-diisopropylethylamine
   DMF N,N-dimethylformamide
   dppf 1,1′-bis(diphenylphosphino)ferrocene
   DMAP 4-dimethylaminopyridine
   DMSO dimethylsulfoxide
   e.e. entantiomeric excess
   EtOAc ethyl acetate
   Et₂O diethyl ether
   GC gas chromatography
   HPLC high-performance liquid chromatography
   hr hours
   LDA Lithium diisopropylamide
   mins minutes
   NMP N-methylpyrrolidone
   o/n overnight
   Pd₂(dba)₃ Tris(dibenzylideneacetone)dipalladium(0)
   iPrOH i-propanol
   rac. racemic
   TBME tert-butylmethyl ether
   TEA triethylamine
   TFA trifluoroacetic acid
   THF tetrahydrofuran
   TMS trimethyl silyl
   Tosyl, Ts para-toluenesulfonyl

### Intermediate 1

### 1-Methyl-4-nitro-1H-imidazole

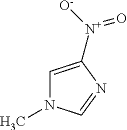

4-Nitro-1H-imidazole (2 g, 17.69 mmol) was dissolved in acetonitrile (20 mL) and potassium carbonate (3.67 g, 26.53 mmol) and iodomethane (1.327 mL, 21.22 mmol) were added. The reaction mixture then heated at 65° C. overnight. The reaction mixture was filtered and the filtrate was concentrated in vacuo leaving a reddish orange solid (3.214 g). This material was purified by ISCO (0-10% MeOH/DCM). Concentration of the fractions in vacuo provided the title product as a yellow solid (2.071 g). The title product was re-crystallized out of isopropanol leaving an off-white solid (1.564 g).

LCMS: 128 [M+H]⁺.

### Intermediate 2

### 5-Fluoropyrimidine-2-carbonitrile

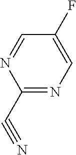

A 10 ml microwave vial was charged with 2-chloro-5-fluoropyrimidine (2.0 g, 15.09 mmol), Pd₂(dba)₃ (0.549 g, 0.6 mmol), dppf (0.67 g, 1.21 mmol), zinc cyanide (1.15 g, 9.81 mmol), and zinc dust (0.237 mg, 3.62 mmol). The flask was evacuated and backfilled with N₂, and anhydrous dimethylacetamide. The vial was mounted onto a Personal Chemistry microwave reactor and heated at 100° C. for 10 hours. The reaction mixture was diluted with EtOAc and then washed with brine three times. The organic layer was obtained and evaporated to dryness. The dried residue was purified by silica gel chromatography (By ISCO Combiflash with gradient EtOAc and hexanes) to afford the title product as a creamy solid (1.50 g, 80%).

¹H NMR (CDCl₃) δ: 8.80 (s, 2H).

GC-MS: 123 [M].

### Intermediate 3

### N-[1-(5-Fluoropyrimidin-2-yl)vinyl]acetamide

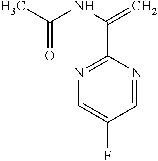

5-Fluoropyrimidine-2-carbonitrile (Intermediate 2, 1.0 g, 8.1 mmol) in THF (10 ml) was added to a solution of MeMgBr (3.3 ml, 9.75 mmol) in ether drop wise at 0° C. After addition, the reaction mixture warmed to room temperature, stirred at room temperature for 1 hour and then diluted with DCM (10 ml). Acetic anhydride (1.23 ml, 13.0 mmol) was added in one portion. The reaction mixture stirred at room temperature for 1 hour and 40° C. for 1 hour. Saturated sodium bicarbonate solution (10 ml) was added and extracted with EtOAc (2×20 ml). The combined organic was dried over sodium sulfate. After removal of solvent, the resulted residue was purified by column chromatography (2.5:1 v/v hexane:EtOAc) to give the title product as a white solid (0.38 g, 26%).

¹H NMR (400 MHz) δ: 9.34 (s, 1H), 8.95 (s, 2H), 6.25 (s, 1H), 6.03 (s, 1H), 2.11 (s, 3H). LCMS: 182 [M+H]⁺ 182.

### Intermediate 4

### N-[(1S)-1-(5-Fluoropyrimidin-2-yl)ethyl]acetamide

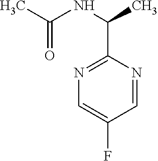

N-[1-(5-Fluoropyrimidin-2-yl)vinyl]acetamide (Intermediate 3, 0.10 g, 0.55 mmol) in MeOH (5 ml) under N₂ was added (+)-1,2-bis((2S,5S)-2,5-diethylphospholano)benzene (cyclooctadiene)rhodium(I) trifluoromethanesulfonate (0.04 g, 0.0055 mmol). The solution was transferred to a high pressure bomb and charged 150 psi H₂. The reaction mixture stirred at room temperature for 4 hours. The solvent was removed and the resulted residue was purified by column chromatography (EtOAc) to give the title product as a white solid (0.096 g, 95%).

¹H NMR (400 MHz) δ: 8.84 (d, 2H), 8.34 (d, 1H), 5.00 (m, 1H), 1.84 (s, 3H), 1.37 (d, 3H).

LCMS: 184 [M+H]⁺.

Enantiomeric excess determined by HPLC (Chiralpak® IA; 95:5 CO₂/MeOH), >99% ee.

### Intermediate 5

### tert-Butyl [(1S)-1-(5-fluoropyrimidin-2-yl)ethyl]carbamate

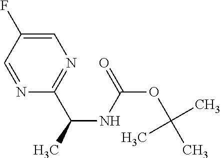

N-[(1S)-1-(5-Fluoropyrimidin-2-yl)ethyl]acetamide (Intermediate 4, 0.20 g, 1.09 mmol), DMAP (0.027 g, 0.22 mmol) and Boc₂O (0.60 g, 2.73 mmol) in THF (10 ml) was stirred at 50° C. for 40 hours. After cooling to room temperature, lithium hydroxide monohydrate (0.094 g, 2.24 mmol) and water (10 ml) was added. The reaction mixture stirred at room temperature for 9 hours. Ether (30 ml) was added, organic layer was separated, washed with brine (20 ml) and dried over sodium sulfate. After removal of solvent, the resulted residue was purified by column chromatography (Hex-EtOAc=5:1) to give the title product as a pale yellow oil (0.21 g, 80%).

¹H NMR (400 MHz) δ: 8.84 (s, 2H), 7.24 (d, 1H), 4.74 (m, 1H), 1.35 (s, 12H).

LCMS: 242 [M+H]⁺.

### Intermediate 6

### (1S)-1-(5-Fluoropyrimidin-2-yl)ethanamine hydrochloride

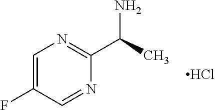

To a solution of tert-butyl [(1S)-1-(5-fluoropyrimidin-2-yl)ethyl]carbamate (Intermediate 5, 0.21 g, 0.87 mmol) in DCM (5 ml) was added HCl (1.3 ml, 5.2 mmol) in dioxane. The reaction mixture stirred at room temperature for 3 hours. The solvent was removed to give the title product as white solid (quantitative).

LCMS: 142 [M+H]⁺.

It should be noted that for those Examples in which Ring A is 5-fluoropyrimidin-2-yl, the carbon bearing the R⁴ substituent may undergo racemization when heated and exposed to a soluble base. This applies as well to the corresponding carbon of Intermediates 37, 38, and 39.

### Intermediate 7

### 2-Chloro-N-(1-methyl-1H-imidazol-4-yl)thieno[2,3-d]pyrimidin-4-amine

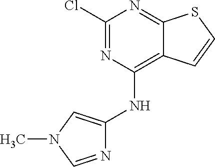

A mixture of 1-methyl-1H-imidazol-4-amine (prepared from Intermediate 1 as described in the synthesis of Intermediate 10, 194 mg, 2 mmol) and 2,4-dichlorothieno[2,3-d]pyrimidine (410 mg, 2.00 mmol) in ethanol (10 mL) was treated with triethylamine (0.279 mL, 2.00 mmol). The resulting mixture was heated at 70° C. overnight. The precipitate was filtered, and washed with ethanol. 303 mg of the title product was obtained.

¹H NMR (300 MHz, MeOD) δ ppm 11.17 (s, 1H) 8.21 (d, 1H) 7.55 (s, 1H) 7.41 (s, 1H) 7.36 (d, 1H) 3.71 (s, 3H).

LCMS: 266 [M+H]⁺.

### Intermediate 8

### 2-Chloro-7-methyl-N-(1-methyl-1H-imidazol-4-yl)thieno[3,2-d]pyrimidin-4-amine

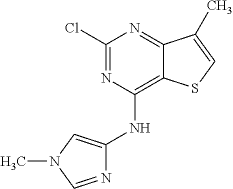

1-Methyl-1H-imidazol-4-amine (prepared from Intermediate 1 as described in the synthesis of Intermediate 10, 194 mg, 2 mmol) and 2,4-dichloro-7-methylthieno[3,2-d]pyrimidine (438 mg, 2.00 mmol) were reacted using a procedure similar to the one described for the synthesis of Intermediate 7, providing the title product (294 mg).

¹H NMR (300 MHz, MeOD) δ ppm 7.85 (s, 1H) 7.53 (s, 1H) 7.40 (s, 1H) 3.71 (s, 3H) 2.30 (s, 3H).

LCMS: 280 [M+H]⁺.

### Intermediate 9

### 2,4-Dichloro-7-[(4-methylphenyl)sulfonyl]-7H-pyrrolo[2,3-d]pyrimidine

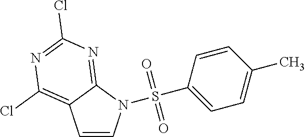

2,4-Dichloro-7H-pyrrolo[2,3-d]pyrimidine (1.00 g, 5.32 mmol), 4-methylbenzene-1-sulfonyl chloride (1.115 g, 5.85 mmol) and tetra-butylammonium hydrogen sulfate (0.090 g, 0.27 mmol) were dissolved in DCM (20 mL) at r.t., and NaOH (50% aq., 1 mL) was added. The reaction mixture stirred at room temperature for 30 minutes. After completion of the reaction as indicated by TLC, the reaction mixture diluted with H₂O and DCM and separated. The organic layer was evaporated in vacuo to obtain a light yellow solid, which was purified by column chromatography (100% DCM) to provide the title product (1.76 g, 97%) as a white solid.

LCMS: 342 [M+H]⁺.

¹H NMR (400 MHz, CHLOROFORM-D) δ ppm 8.14 (d, J=8.59 Hz, 2H) 7.78 (d, J=3.79 Hz, 1H) 7.39 (d, J=8.59 Hz, 2H) 6.70 (d, J=3.79 Hz, 1H) 2.45 (s, 3H).

### Intermediate 10

### 2-Chloro-N-(1-methyl-1H-imidazol-4-yl)-7-[(4-methylphenyl)sulfonyl]-7H-pyrrolo[2,3-d]pyrimidin-4-amine

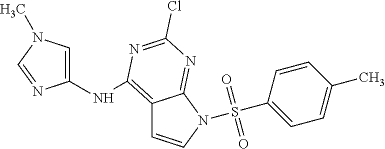

1-Methyl-4-nitro-1H-imidazole (Intermediate 1, 50 mg, 0.39 mmol) was dissolved in ethanol (5 mL) and Pd/C (5 wt %, Degussa®, 20.93 mg, 9.83 μmol) was added. The reaction mixture stirred under 1 atm of hydrogen at r.t. for 3 hours and was then filtered through diatomaceous earth (Celite® brand) to give 1-methyl-1H-imidazol-4-amine. 2,4-Dichloro-7-[(4-methylphenyl)sulfonyl]-7H-pyrrolo[2,3-d]pyrimidine (Intermediate 9, 108 mg, 0.31 mmol) and TEA (0.110 mL, 0.79 mmol) was added to the reaction mixture. The reaction mixture was stirred at 100° C. in a microwave reactor for 2 hr. After completion of the reaction as indicated by TLC, the reaction mixture was evaporated in vacuo to obtain a light yellow solid, which was purified by column chromatography (3% MeOH, 0.3% NH₄OH in DCM) to provide the title product (90 mg, 57%) as a white solid.

LCMS: 403 [M+H]⁺.

¹H NMR (400 MHz, CHLOROFORM-D) δ ppm 8.92 (s, 1H) 8.03 (d, J=8.34 Hz, 2H) 7.41 (s, 1H) 7.39 (d, J=3.79 Hz, 1H) 7.25 (d, J=8.08 Hz, 2H) 6.48 (s, 1H) 3.67 (s, 3H) 2.33 (s, 3H).

The title product was also prepared according to the following method:

A solution of 1-methyl-1H-imidazol-4-amine hydrochloride (Intermediate 36, 16.39 g, 122.74 mmol) and 2,4-dichloro-7-[(4-methylphenyl)sulfonyl]-7H-pyrrolo[2,3-d]pyrimidine (Intermediate 9, 21 g, 61.37 mmol) and DIPEA (42.9 ml, 245.47 mmol) in ethanol (264 ml) were heated at 88° C. overnight. The reaction mixture was cooled to 0° C. and filtered to provide 2-chloro-N-(1-methyl-1H-imidazol-4-yl)-7-tosyl-7H-pyrrolo[2,3-d]pyrimidin-4-amine contaminated with DIPEA. The solid was dissolved in EtOAc (400 ml) and the solution was washed with water (3×100 ml). During the process, the title product crashed out of solution and collected through filtration. Concentration of the mother liquor provided additional title product (total=18.8 g, 76%). LCMS: 403 [M+H]

¹H NMR (300 MHz, DMSO-d6) δ ppm 10.75 (br. s., 1H), 7.96 (d, 2H), 7.63 (d, 1H), 7.40-7.55 (m, 3H), 7.35 (s, 1H), 7.23 (br. s., 1H), 3.68 (s, 3H), 2.37 (s, 3H)

### Intermediate 11

### 2,4-Dichloro-5-[(4-methylphenyl)sulfonyl]-5H-pyrrolo[3,2-d]pyrimidine

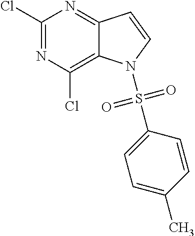

2,4-Dichloro-5H-pyrrolo[3,2-d]pyrimidine (500 mg, 2.66 mmol) and 4-methylbenzene-1-sulfonyl chloride (558 mg, 2.93 mmol) were reacted using a procedure similar to the one described for the synthesis of Intermediate 9, providing the title product.

LCMS: 342 [M+H]⁺.

¹H NMR (400 MHz, CHLOROFORM-D) δ ppm 8.34 (d, J=3.79 Hz, 1H) 7.75 (d, J=8.59 Hz, 2H) 7.34 (d, J=8.08 Hz, 2H) 6.87 (d, J=3.79 Hz, 1H) 2.44 (s, 3H).

### Intermediate 12

### 2-Chloro-N-(1-methyl-1H-imidazol-4-yl)-5-[(4-methylphenyl)sulfonyl]-5H-pyrrolo[3,2-d]pyrimidin-4-amine

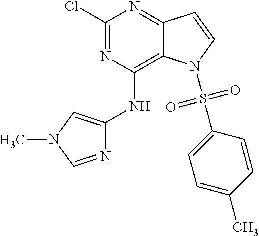

2,4-Dichloro-5-[(4-methylphenyl)sulfonyl]-5H-pyrrolo[3,2-d]pyrimidine (Intermediate 11, 240 mg, 0.70 mmol) and 1-methyl-1H-imidazol-4-amine (1.5 eq., prepared from Intermediate 1 as described in the synthesis of Intermediate 10), were reacted using a procedure analogous to that described for the synthesis of Intermediate 10, providing the title product (90 mg).

LCMS: 403 [M+H]⁺.

¹H NMR (400 MHz, CHLOROFORM-D) δ ppm 9.92 (s, 1H) 7.71 (d, J=3.79 Hz, 1H) 7.61 (d, J=8.59 Hz, 2H) 7.44 (br.s, 1H) 7.24 (s, 1H) 7.16 (d, J=8.08 Hz, 2H) 6.62 (d, J=3.79 Hz, 1H) 3.68 (s, 3H) 2.28 (s, 3H).

### Intermediate 13

### 5-Chloro-2-methyl-N-(1-methyl-1H-imidazol-4-yl) [1,3]thiazolo[5,4-d]pyrimidin-7-amine

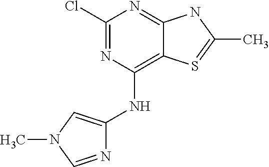

A mixture of 5,7-dichloro-2-methyl[1,3]thiazolo[5,4-d]pyrimidine (Intermediate 16, 380 mg, 1.73 mmol), DIPEA (0.754 mL, 4.32 mmol) and 1-methyl-1H-imidazol-4-amine (prepared from Intermediate 1 as described in the synthesis of Intermediate 10, 201 mg, 2.07 mmol) in EtOH (15 mL) was heated for 1 hour at 70° C., LCMS analysis indicated the reaction was complete. The title product (400 mg) was obtained after filtration and was used in a subsequent step without any further purification.

LCMS: 281 [M+H]⁺.

¹H NMR (300 MHz, DMSO-d6) δ ppm 10.29 (s, 1H), 7.50 (d, J=1.32 Hz, 1H), 7.37 (d, J=1.51 Hz, 1H), 3.70 (s, 3H), 2.83 (s, 3H).

### Intermediate 14

### 2-Chloro-N-(1-methyl-1H-imidazol-4-yl)-6,7-dihydro-5H-cyclopenta[d]pyrimidin-4-amine

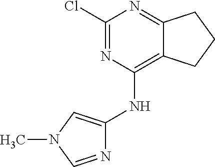

A mixture of 2,4-dichloro-6,7-dihydro-5H-cyclopenta[d]pyrimidine (321 mg, 1.70 mmol), DIPEA (0.89 mL, 5.1 mmol) and 1-methyl-1H-imidazol-4-amine (prepared from Intermediate 1 as described in the synthesis of Intermediate 10, 200 mg, 2.04 mmol) in EtOH (15 mL) was heated overnight at 70° C. LCMS analysis indicated that the reaction was complete. The title product (350 mg) was obtained after filtration and was used in a subsequent step without any further purification.

¹H NMR (300 MHz, DMSO-d6) δ ppm 9.72 (s, 1H), 7.46 (d, J=1.32 Hz, 1H), 7.30 (d, J=1.51 Hz, 1H), 3.67 (s, 3H), 2.76 (m, 4H), 2.02 (dq, J=7.72, 7.54 Hz, 2H).

LCMS: 250.1 [M+H]⁺.

### Intermediate 15

### 5-Amino-2-methyl-1,3-thiazole-4-carbonitrile

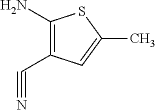

To a stirred solution of aminomalonitrile para-toluenesulfonate salt (2 g) in pyridine (15 mL) was added ethyl ethane(dithioate) (0.68 g) drop-wise at room temperature. The reaction mixture was stirred at this temperature overnight. The volatiles were evaporated under reduced pressure and purification by column chromatography afforded the title product (2.2 g).

¹H NMR (400 MHz) δ: 2.48 (s, 3H).

### Intermediate 16

### 5,7-Dichloro-2-methyl[1,3]thiazolo[5,4-d]pyrimidine

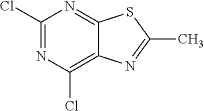

To a stirred solution from 5-amino-2-methyl-1,3-thiazole-4-carbonitrile (Intermediate 15) in MeCN (3 mL) was added diphosgene drop-wise at 0° C. The solution was stirred at 130° C. for 1 hour. The volatiles were evaporated under reduced pressure and purification by column chromatography afforded the title product.

LCMS: 220 [M+H]⁺.

¹H NMR (400 MHz, CDCl₃) δ: 2.93 (s, 3H).

### Intermediate 17

### 4-Chloro-1-Ethyl-6-(methylsulfanyl)-1H-pyrazolo[3,4-d]pyrimidine

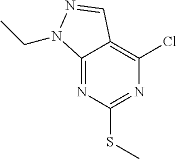

4,6-Dichloro-2-(methylthio)pyrimidine-5-carbaldehyde (500 mg, 2.24 mmol) and ethylhydrazine oxalate (336 mg, 2.24 mmol) were dissolved in ethanol (6.222 mL) and TEA (1.250 mL, 8.97 mmol) was added. The reaction was stirred at rt for 1 hour. The reaction mixture was concentrated in vacuo leaving a yellow solid. This material was separated between EtOAc and water, washed with brine, aq. NaHCO₃ and dried with MgSO₄. Concentration in vacuo provided the title product as a yellow solid (480 mg).

LCMS: 229 [M+H]⁺.

### Intermediate 18

### 4-Chloro-1-ethyl-6-(methylsulfonyl)-1H-pyrazolo[3,4-d]pyrimidine

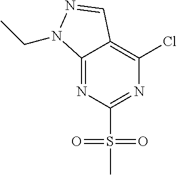

4-Chloro-1-ethyl-6-(methylsulfanyl)-1H-pyrazolo[3,4-d]pyrimidine (Intermediate 17, 480 mg, 2.10 mmol) was dissolved in DCM (10.500 mL) and mCPBA (1.411 g, 6.30 mmol) was added portion-wise. The reaction mixture was stirred at rt for 2 hours. The volatiles were removed in vacuo leaving a pale yellow solid. This material was purified by ISCO (15%→50% EtOAc/Hexanes). Concentration of the fractions in vacuo provided the title product as a pale yellow solid (478 mg).

LCMS: 261 [M+H]⁺.

### Intermediate 19

### 1-Ethyl-N-(1-methyl-1H-imidazol-4-yl)-6-(methylsulfonyl)-1H-pyrazolo[3,4-d]pyrimidin-4-amine

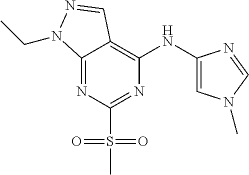

1-Methyl-1H-imidazol-4-amine hydrochloride (Intermediate 36, 245 mg, 1.83 mmol) was dissolved in ethanol (5.090 mL) at 0° C. and TEA (1.022 mL, 7.33 mmol) and 4-chloro-1-ethyl-6-(methylsulfonyl)-1H-pyrazolo[3,4-d]pyrimidine (Intermediate 18, 478 mg, 1.83 mmol) were added. The reaction was slowly allowed to warm to rt overnight. The reaction mixture was filtered providing the title product as an off-white solid (395 mg).

LCMS: 322 [M+H]⁺.

### Intermediate 20

### 2,4-Dichloropteridine

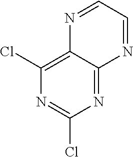

A mixture of pteridine-2,4-diol (0.517 g, 3.15 mmol), POCl₃ (5.17 ml, 55.47 mmol) and PCl₅ (2.62 g, 12.60 mmol) was refluxed at 110° C. for 2 hours. The reaction mixture was cooled to rt and concentrated in vacuo (using toluene as an azeotrope) providing the title product as a red residue.

LCMS: 202 [M+H]⁺.

### Intermediate 21

### 2-Chloro-N-(1-methyl-1H-imidazol-4-yl)pteridin-4-amine

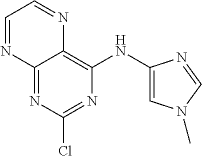

1-Methyl-4-nitro-1H-imidazole (Intermediate 1, 400 mg, 3.15 mmol) was dissolved in ethanol (4.540 mL) and Pd/C (10 wt %, Degussa®) (84 mg, 0.08 mmol) was added. The reaction was subjected to an atmosphere of hydrogen (1 atm) (63.4 mg, 31.47 mmol) for 3 hours. The reaction mixture was filtered through diatomaceous earth (Celite® brand) and TEA (1.755 mL, 12.59 mmol) was added to the filtrate followed by 2,4-dichloropteridine (Intermediate 20, 633 mg, 3.15 mmol). The reaction was heated at 70° C. overnight and was subsequently concentrated in vacuo leaving a rust-colored solid (5.828 g). This material was purified by ISCO (3-15% MeOH/DCM). Concentration of the fractions in vacuo provided the title product as an orange solid (135 mg).

LCMS: 262 [M+H]⁺.

### Intermediate 22

### 6-Chloro-1-methyl-N-(1-methyl-1H-imidazol-4-yl)-1H-pyrazolo[3,4-d]pyrimidin-4-amine

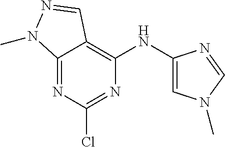

4,6-Dichloro-1-methyl-1H-pyrazolo[3,4-d]pyrimidine (4.492 g, 22.12 mmol) and 1-methyl-1H-imidazol-4-amine hydrochloride (Intermediate 36, 2.96 g, 22.12 mmol) were suspended in ethanol (104 ml) and TEA (6.17 ml, 44.25 mmol) was added. The reaction mixture was then heated at 70° C. overnight. The reaction mixture was cooled to 0° C. and filtered providing the title product as a purple/grey solid (2.940 g).

LCMS: 264[M+H]⁺.

### Intermediate 23

### 2-Chloro-N-(1-methyl-1H-imidazol-4-yl)pyrido[2,3-d]pyrimidin-4-amine

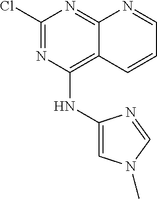

1-Methyl-1H-imidazol-4-amine hydrochloride (Intermediate 36, 167 mg, 1.72 mmol), 2,4-dichloropyrido[2,3-d]pyrimidine (500 mg, 2.50 mmol) were suspended in ethanol (10 mL) and TEA (0.24 mL, 1.72 mmol) was added. The reaction mixture was heated at 70° C. overnight and the title product was obtained after filtration (421 mg).

¹H NMR (300 MHz, DMSO-d6) δ ppm 11.33 (s, 1H) 9.16 (d, 1H) 9.01 (s, 1H) 7.54-7.72 (m, 3H) 3.75 (s, 3H).

LCMS: 261 [M+H]⁺.

### Intermediate 24

### 2-Amino-6-(trifluoromethyl)nicotinic acid

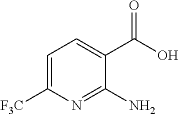

A solution of 2-chloro-6-(trifluoromethyl)nicotinic acid (1.87 g, 8.29 mmol) in (2,4-dimethoxyphenyl)methanamine (2.491 ml, 16.58 mmol) was heated to 100° C. overnight. The reaction mixture was concentrated under vacuum and partitioned between water and DCM. Evaporation of the organic layer provided a dark brown residue, which was dissolved in TFA (2.55 ml, 33.16 mmol), and the resulting mixture was stirred for 30 minutes. The precipitate formed was discarded via filtration and concentration of the filtrate under reduced pressure gave a residue. This residue was dissolved in HCl (1N, 200 mL) and the aqueous solution was washed with Et₂O and evaporated under reduced pressure to give a solid. This solid was washed with DCM/Hexanes, dried in a vacuum oven overnight and characterized as the title product (2 g).

LCMS: 207.0 [M+H]⁺.

### Intermediate 25

### 2-{4-[(1-Methyl-1H-imidazol-4-yl)amino]-6-(methylsulfonyl)-1H-pyrazolo[3,4-d]pyrimidin-1-yl}ethanol

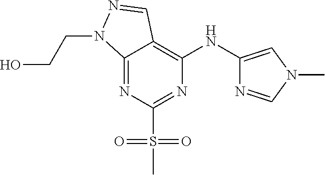

To a solution of 2-{4-[(1-methyl-1H-imidazol-4-yl)amino]-6-(methylsulfanyl)-1H-pyrazolo[3,4-d]pyrimidin-1-yl}ethanol (Intermediate 26, 305 mg, 1.00 mmol) in DCM (5 mL), mCPBA (448 mg, 2.00 mmol) was added portion-wise at 0° C. The resulting mixture was allowed to warm to ambient temperature and stirred for 30 minutes. The mixture was separated between ethyl acetate/MeOH (90:10 v/v) and aq. potassium carbonate solution. The organic layer was dried over MgSO₄ and the volatiles were evaporated under reduced pressure. The title product was used in the subsequent step without any further purification.

LCMS: 338 [M+H]⁺.

### Intermediate 26

### 2-{4-[(1-Methyl-1H-imidazol-4-yl)amino]-6-(methylsulfanyl)-1H-pyrazolo[3,4-d]pyrimidin-1-yl}ethanol

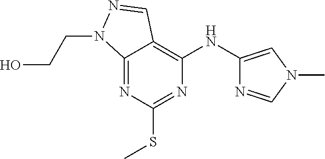

To a solution of 1-methyl-4-nitro-1H-imidazole (Intermediate 1, 528 mg, 4.15 mmol) in ethanol (20 mL), was added palladium on carbon (100 mg, 0.09 mmol) and the mixture was subjected to an atmosphere of hydrogen for 3 hours. The mixture was filtered and 2-[4-chloro-6-(methylsulfanyl)-1H-pyrazolo[3,4-d]pyrimidin-1-yl]ethanol (Intermediate 27, 847 mg, 3.46 mmol) followed by triethylamine (0.723 mL, 5.19 mmol) was added in the filtrate. The resulting mixture was heated at 70° C. overnight. The volatiles were removed under reduced pressure to give a residue. Purification (ISCO) provided the title product (810 mg).

LCMS: 306 [M+H]⁺.

### Intermediate 27

### 2-[4-Chloro-6-(methylsulfanyl)-1H-pyrazolo[3,4-d]pyrimidin-1-yl]ethanol

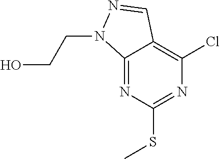

To a solution of 4,6-dichloro-2-(methylthio)pyrimidine-5-carbaldehyde (500 mg, 2.24 mmol) in THF were added triethylamine (0.469 mL, 3.36 mmol) and 2-hydrazinylethanol (0.152 mL, 2.24 mmol, drop-wise). The reaction mixture was allowed to stir at room temperature overnight. The volatiles were removed under reduced pressure to give the title product (0.429 g) that was used in the subsequent step without any further purification.

LCMS: 245 [M+H]⁺.

### Intermediate 28

### tert-Butyl 2-chloro-4-[(1-methyl-1H-imidazol-4-yl)amino]-7,8-dihydropyrido[4,3-d]pyrimidine-6(5H)-carboxylate

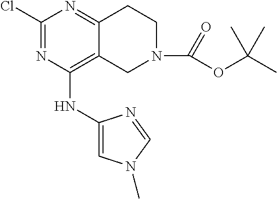

1-Methyl-1H-imidazol-4-amine hydrochloride (Intermediate 36, 194 mg, 2.0 mmol), tert-butyl 2,4-dichloro-7,8-dihydropyrido[4,3-d]pyrimidine-6(5H)-carboxylate (0.608 g, 2.00 mmol) were reacted using a procedure similar to the one described for the synthesis of Intermediate 23, providing the title product (428 mg).

LCMS: 365 [M+H]⁺.

### Intermediate 29

### tert-Butyl 2-chloro-4-[(1-methyl-1H-imidazol-4-yl)amino]-5,7-dihydro-6H-pyrrolo[3,4-d]pyrimidine-6-carboxylate

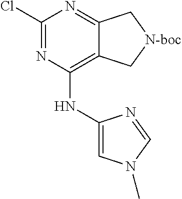

1-Methyl-1H-imidazol-4-amine hydrochloride (Intermediate 36, 167 mg, 1.72 mmol) and tert-butyl 2,4-dichloro-5H-pyrrolo[3,4-d]pyrimidine-6(7H)-carboxylate (500 mg, 1.72 mmol) were reacted using a procedure similar to the one described for the synthesis of Intermediate 23, providing the title product (467 mg).

¹H NMR (300 MHz, MeOD) δ ppm 7.48 (s, 1H) 7.32 (s, 1H) 4.50 (s, 2H) 4.41 (s, 2H) 3.68 (s, 3H) 1.46 (s, 9H).

LCMS: 351 [M+H]⁺.

### Intermediate 30

### 1-(3,5-Difluoropyridin-2-yl)-2-methoxyethanone

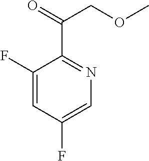

3,5-Difluoropyridine (5.0 g, 43.45 mmol) in THF was cooled to −72° C. (external −80° C.). LDA (23.9 mL, 1.1 eq.) was added drop-wise at such rate that the internal temp did not increase more than 3° C. during addition. The reaction mixture turned into a deep brownish, thick phase and was stirred for 30 minutes at this temperature. TMS-Cl (43.4 mL, 43.45 mmol) was added via syringe in a relatively fast fashion. The reaction became a clear and light yellow solution. LDA (23.9 mL, 1.1 eq.) was added drop-wise in a quicker version, and the reaction mixture was allowed to stir for 2 hours. Methyl 2-methoxyacetate (5.59 mL, 56.48 mmol) was added quickly through a syringe. The reaction mixture was quenched at −78° C. by adding 20 ml of saturated NH₄Cl solution. Evaporation of the organic extracts under reduced pressure gave a colored residue. Purification utilizing ISCO (0-→25% EtOAc/hexanes), gave the title product (3 g).

LCMS: 188 [M+H]⁺.

### Intermediate 31

### 1-(3,5-Difluoropyridin-2-yl)-2-methoxyethanone oxime

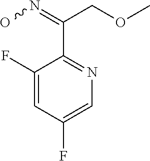

1-(3,5-Difluoropyridin-2-yl)-2-methoxyethanone (Intermediate 30) dissolved in ethanol (255 ml). Hydroxylamine hydrochloride (14.22 g, 204.61 mmol) was added, followed by drop-wise addition of triethylamine (28.5 ml, 204.61 mmol). The resulting colored mixture was heated to 50° C. for 2 hours. The volatiles were evaporated under reduced pressure and the residue left was partitioned between water (255 ml) and ethyl acetate (255 ml). The separated aqueous layer was further extracted into 2× ethyl acetate (255 ml). The combined organic extracts washed with water (255 ml), saturated brine (255 ml), dried over MgSO₄, filtered and concentrated in vacuo to give 42 g of a brown oil. Purification by column chromatography (25%→40% EtOAc in isohexanes) gave 32 g of the title product as a yellow oily solid (˜3:1 mixture of isomers).

Trituration in MTBE gave the title product (12.3 g, 60.84 mmol, 44.6%, single isomer) as a white solid. The liquor was evaporated under reduced pressure and the residue was re-columned using the conditions described previously, followed by trituration with EtOAc/isohexanes, giving additional title product (7.2 g, 35.62 mmol, 26.1%).

LCMS: 203 [M+H]⁺.

### Intermediate 32

### (1R)-1-(3,5-Difluoropyridin-2-yl)-2-methoxyethanamine, (R)-mandelic acid salt

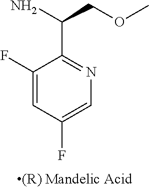

1-(3,5-Difluoropyridin-2-yl)-2-methoxyethanone oxime (Intermediate 31) was dissolved in EtOAc (0.4M) and was subsequently subjected to catalytic hydrogenation (Pd on C) in a Parr Hydrogenator (Pressure 5 bar at 40° C.) for 1 hour. The catalyst was filtered via diatomaceous earth (Celite® brand) and the filtrate of 1-(3,5-difluoropyridin-2-yl)-2-methoxyethanamine (0.4 M in ethyl acetate) (180 mL, 72.00 mmol) was treated with (R)-Mandelic acid (5.81 g, 38.16 mmol). Precipitation was observed almost instantaneously and the resulting mixture was allowed to stir o/n. The title product was collected via filtration (8.5 g, 69.4%).

¹H NMR (400 MHz) δ ppm 8.6 (s, 1H) 8.01 (m, 1H) 7.41 (t, 2H) 7.36 (t, 2H) 7.19 (m, 1H) 4.81 (s, 1H) 4.50 (m, 1H) 3.57 (d, 2H) 3.23 (s, 3H).

LCMS: 188 [M−H]⁺.

### Intermediate 33

### 1-(3,5-Difluoropyridin-2-yl)ethanone

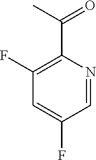

A solution of methylmagnesium bromide (36.8 ml, 117.78 mmol) in THF (50 ml) was stirred under N₂ and cooled to −78° C. 3,5-difluoropicolinonitrile (15.0 g, 107.07 mmol) in THF (50 ml) was added drop wise with an addition funnel at such a rate that the internal temperature was kept below −4° C. After the addition was complete, the reaction mixture was poured into a 1M HCl (100 ml, chilled in an ice bath). The reaction mixture was stirred at 0° C. for 30 minutes and at room temperature for 30 minutes. To this solution 150 ml of EtOAc was added to extract product. The aquous phase was neutralized to pH9 with NaHCO₃ and extracted with EtOAc (2×20 ml). The organic layers were combined and the volatiles were removed under reduced pressure. Purification utilizing ISCO (0-10% EtOAc-hexanes) gave the title product as light yellow oil.

LC-MS: 158 [M+H]⁺.

### Intermediate 34

### 1-(3,5-Difluoropyridin-2-yl)ethanone oxime

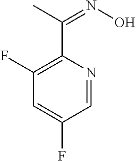

To a solution of 1-(3,5-difluoropyridin-2-yl)ethanone (Intermediate 33, 12.91 g, 82.17 mmol) in ethanol (164 ml) was added hydroxylamine hydrochloride (8.56 g, 123.25 mmol) followed by Et₃N (17.18 ml, 123.25 mmol) and the resulting mixture was stirred o/n at r.t. The volatiles were removed under reduced pressure and the resulting residue was partitioned between EtOAc/H₂O. The organic extracts washed with brine and dried. Orange yellow solid was obtained and purification utilizing ISCO (10% EtOAc/hexanes→25% EtOAc/hexanes) gave the title product (9.73 g, 68.8%) as yellow solid.

¹H NMR (300 MHz, DMSO-d₆) δ ppm 2.19 (s, 3H) 7.98 (ddd, J=10.97, 8.81, 2.26 Hz, 1H) 8.55 (d, J=2.26 Hz, 1H) 11.70 (s, 1H).

LC-MS: 173 [M+H]⁺.

### Intermediate 35

### 1-(3,5-Difluoropyridin-2-yl)ethanamine hydrochloride

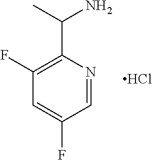

1-(3,5-Difluoropyridin-2-yl)ethanone oxime (Intermediate 34, 9.73 g, 56.53 mmol) was added to water (113 ml) to form a suspension. Ammonium hydroxide (22.01 ml, 565.26 mmol) was added to the above solution, followed by ammonium acetate (5.23 g, 67.83 mmol). The mixture was heated at 50° C. and subsequently zinc (14.79 g, 226.11 mmol) was added portion wise while maintaining the internal temperature below 65° C.

After the addition was complete, the reaction mixture was stirred at 50° C. for 3 hr. Solid NaCl and EtOAc was added to quench the reaction, stirred for 1 hr at r.t., was then filtered through diatomaceous earth (Celite® brand) and rinsed with EtOAc. The organic layer was washed with 5 ml 2.5% NaOH (aq.) then 10 ml NH₄OH. The organic layer was then washed with brine and dried with Na₂SO₄. The organic layer was concentrated under reduced pressure to obtain the title product as light yellow oil.

¹H NMR (400 MHz, MeOD) δ ppm 1.62 (d, J=6.82 Hz, 3H) 4.86 (q, J=6.82 Hz, 1H) 7.75 (ddd, J=10.11, 8.34, 2.27 Hz, 1H) 8.49 (d, J=2.27 Hz, 1H).

The hydrochloride salt was obtained after stirring the parent compound in MeOH in the presence of HCl (4N in dioxane) for 1 hour and subsequently evaporating the volatiles under reduced pressure.

### Intermediate 36

### 1-Methyl-1H-imidazol-4-amine hydrochloride

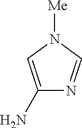

1-Methyl-4-nitro-1H-imidazole (25 g, Intermediate 1) was dissolved in EtOH (800 ml) and Pd(OH)₂ (2.5 g) was added. The mixture was subjected to an atmosphere of hydrogen for 3 hours at room temperature. The mixture was filtered and the organic layer was concentrated to give the 1-methyl-1H-imidazol-4-amine. The amine was dissolved in EtOH (800 ml) and stirred at room temperature. A saturated solution of EtOH with HCl gas (750 ml) was added. The mixture was stirred for 30 minutes and the EtOH was concentrated under reduced pressure to 100 ml, filtered, and washed with ether to give the title product (28.4 g).

LCMS: 98 [M+H]⁺.

### Intermediate 37

### N²-[(1S)-1-(5-fluoropyrimidin-2-yl)ethyl]-N⁴-(1-methyl-1H-imidazol-4-yl)pyrimidine-2,4,6-triamine

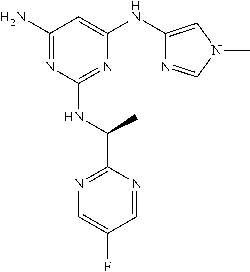

N⁴-(Diphenylmethylene)-N²-[(1S)-1-(5-fluoropyrimidin-2-yl)ethyl]-N⁶-(1-methyl-1H-imidazol-4-yl)pyrimidine-2,4,6-triamine (Intermediate 38, 2122 mg, 4.3 mmol) in THF (13 mL) was treated with aq. HCl solution (8600 μl, 17.20 mmol, 2N aq). After stirring for 2 h, the reaction mixture was diluted with water. The aqueous layer was washed with EtOAc and was neutralized to pH=10 using aq. NaOH (1N). The aqueous layer was extracted with DCM/MeOH (10%, 3×). The combined organic layers were evaporated under reduced pressure leaving a residue, which was purified utilizing ISCO (0→10% DCM/MeOH/1% ammonia hydroxide) to provide the title product as part of a mixture of enantiomers (400 mg, 28.2%), the title enantiomer being present in the mixture in an amount greater than or equal to the amount of the corresponding R enantiomer.

LCMS: 330 [M+H]⁺.

¹H NMR (300 MHz, DMSO-d6) δ ppm 8.74-8.90 (m, 2H), 8.59 (s, 1H), 7.25 (d, J=1.13 Hz, 1H), 7.13 (br. s., 1H), 6.27 (d, 1H), 5.61 (br.s, 2H), 5.23 (q, 1H), 5.18 (s, 1H), 3.61 (s, 3H), 1.46 (d, 3H).

### Intermediate 38

### N⁴-(Diphenylmethylene)-N²-[(1S)-1-(5-fluoropyrimidin-2-yl)ethyl]-N⁶-(1-methyl-1H-imidazol-4-yl)pyrimidine-2,4,6-triamine

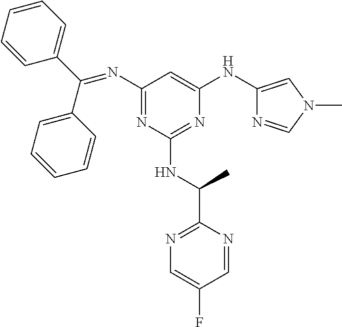

A solution of 6-Chloro-N²-[(1S)-1-(5-fluoropyrimidin-2-yl)ethyl]-N⁴-(1-methyl-1H-imidazol-4-yl)pyrimidine-2,4-diamine (Intermediate 39, 1.5 g, 4.30 mmol), Pd₂dba₃ (0.276 g, 0.30 mmol), BINAP (0.402 g, 0.65 mmol), and CS₂CO₃ (6.31 g, 19.35 mmol) was heated at 110° C. in DMA (20.07 ml) overnight. The reaction mixture was diluted with DCM and washed with brine. Concentration of organic layer under reduced pressure provided a residue, which was purified utilizing ISCO (100% EtOAc then 5%→15% MeOH/DCM) to yield the title product as part of a mixture of enantiomers, the title enantiomer being present in the mixture in an amount greater than or equal to the amount of the corresponding R enantiomer.

LCMS: 493 [M+H]⁺.

### Intermediate 39

### 6-Chloro-N²-[(1S)-1-(5-fluoropyrimidin-2-yl)ethyl]-N⁴-(1-methyl-1H-imidazol-4-yl)pyrimidine-2,4-diamine

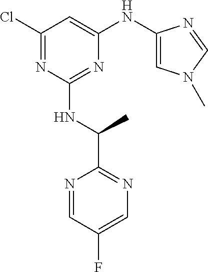

2,6-Dichloro-N-(1-methyl-1H-imidazol-4-yl)pyrimidin-4-amine (Intermediate 40, 244 mg, 1.00 mmol), (1S)-1-(5-fluoropyrimidin-2-yl)ethanamine hydrochloride (Intermediate 6, 213 mg, 1.20 mmol), DIPEA (0.436 ml, 2.50 mmol) in n-BuOH (2 ml) and NMP (0.5 ml) was heated at 90° C. for 24 hours. LCMS indicated complete conversion. The volatiles were removed under reduced pressure and the derived residue was purified utilizing ISCO to afford the title product as part of a mixture of enantiomers (287 mg, 82%), the title enantiomer being present in the mixture in an amount greater than or equal to the amount of the corresponding R enantiomer.

¹H NMR (400 MHz, DMSO-d₆) δ ppm 9.65 (br. s., 1H) 8.86 (s, 2H) 7.76 (br. s., 1H) 7.32 (br. s., 1H) 7.01 (br. s, 1H) 6.01 (br. s., 1H) 5.16 (m, 1H) 3.64 (s, 3H) 1.49 (d, 3H).

LCMS: 349 [M+H]⁺.

### Intermediate 40

### 2,6-Dichloro-N-(1-methyl-1H-imidazol-4-yl)pyrimidin-4-amine

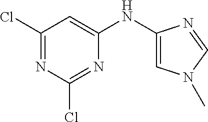

1-Methyl-4-nitro-1H-imidazole (Intermediate 1, 1.0 g, 7.87 mmol) was dissolved in ethanol (12.82 ml) and Pd/C (10 wt %, Degussa®, 0.209 g, 0.20 mmol) was added. The reaction was subjected to 1 atm of hydrogen for 3 hours. TLC analysis indicated that the reaction was completed and the reaction mixture was filtered through diatomaceous earth (Celite® brand) and cooled to 0° C. TEA (2.193 ml, 15.74 mmol) and 2,4,6-trichloropyrimidine (0.722 ml, 6.29 mmol) were added and the reaction was allowed to slowly warm at rt overnight. LCMS confirmed formation of the desired product. The reaction mixture was then filtered leaving a tan solid (1.526 g), which was confirmed by LCMS to be the title product with 99% purity. The material was used in a subsequent step without any further purification.

LCMS: 245 [M+H]⁺.

### Intermediate 41

### 2-Chloro-6-methyl-N-(1-methyl-1H-imidazol-4-yl)-7-[4-methylphenyl)sulfonyl]-7H-pyrrolo[2,3-d]pyrimidin-4-amine

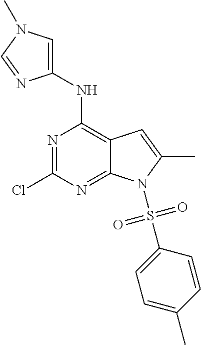

A mixture of 1-methyl-4-nitro-1H-imidazole (Intermediate 1, 0.963 g, 7.58 mmol) and Pd on charcoal (0.19 g, 0.18 mmol) in ethanol (7.10 ml) was placed under H₂. After filtration through diatomaceous earth (Celite® brand), the filtrate was added to 2,4-dichloro-6-methyl-7-[(4-methylphenyl)sulfonyl]-7H-pyrrolo[2,3-d]pyrimidine (Intermediate 42, 0.9 g, 2.53 mmol) and DIPEA (1.324 ml, 7.58 mmol) and the resulting mixture stirred overnight at 90° C. The reaction mixture was diluted with water and extracted with DCM/MeOH (10%). Evaporation of the volatiles under reduced pressure gave a residue, which was purified utilizing ISCO (0%→100% Hexanes/EtOAc then 0%→10% MeOH/DCM) to provide the title product (680 mg). LCMS: 417 [M+H]⁺.

### Intermediate 42

### 2,4-Dichloro-6-methyl-7-[(4-methylphenyl)sulfonyl]-7H-pyrrolo[2,3-d]pyrimidine

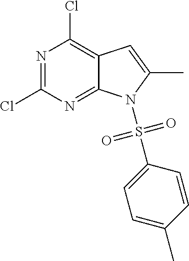

A solution of 2,4-dichloro-7-[(4-methylphenyl)sulfonyl]-7H-pyrrolo[2,3-d]pyrimidine (Intermediate 9, 1 g, 2.92 mmol) in THF (10.76 ml) was treated with LDA (3.65 ml, 7.31 mmol) at −78° C. After the reaction was stirred for 1 hour at this temperature, MeI (0.201 ml, 3.21 mmol) was added to the solution. Reaction was kept at −78° C. and stirred for an extra 3 hours at this temperature. The reaction mixture was poured into aqueous ammonia chloride solution and extracted with EtOAc. The organic extract was concentrated under reduced pressure to give a residue, which was purified utilizing ISCO (0%→100% Hexanes/DCM) to yield the title product (0.200 g).

LCMS: 357 [M+H]⁺.

### Intermediate 43

### 2-Chloro-7-(2-fluoroethyl)-N-(1-methyl-1H-imidazol-4-yl)-7H-pyrrolo[2,3-d]pyrimidin-4-amine

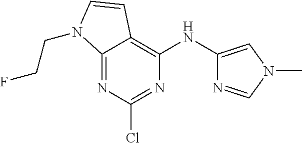

The solution of 2,4-dichloro-7-(2-fluoroethyl)-7H-pyrrolo[2,3-d]pyrimidine (Intermediate 44, 600 mg, 2.56 mmol) and 1-methyl-1H-imidazol-4-amine hydrochloride (Intermediate 36, 523 mg, 3.08 mmol) was added to DIPEA (2686 μl, 15.38 mmol) in ethanol (5859 μl) and the reaction mixture was heated at 90° C. for 24 hours. Additional 1-methyl-1H-imidazol-4-amine hydrochloride (Intermediate 36, 523 mg, 3.08 mmol) and DIPEA (2686 μl, 15.38 mmol) added to the reaction mixture and the mixture was heated at 90° C. for another 24 hours. The volatiles were removed under reduced pressure to afford a residue, which was dissolved in DCM/MeOH (10%) and washed with water. The organic layer was concentrated in vacuum, followed by purification by reversed phase HPLC (Gilson® chromatography, 0%→50% MeCN/0.1% TFA H₂O) to yield the title product (454 mg).

LCMS: 297 [M+H]⁺.

### Intermediate 44

### 2,4-Dichloro-7-(2-fluoroethyl)-7H-pyrrolo[2,3-d]pyrimidine

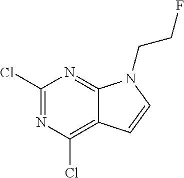

2,4-Dichloro-7H-pyrrolo[2,3-d]pyrimidine (1000 mg, 5.32 mmol) was dissolved in acetonitrile (3550 μl) and sodium hydride (319 mg, 7.98 mmol) was added portion-wise. The reaction mixture was stirred at room temperature for 30 minutes until gas evolution ceased. 1-Bromo-2-fluoroethane (1519 mg, 11.97 mmol) was added and the resulting mixture was stirred for 30 minutes. The reaction mixture was then poured into water and extracted with DCM/MeOH. Concentration of the organic layers under reduced pressure provided a residue, which was purified utilizing ISCO (0%-→100% EtOAc/Hexanes) to afford the title product (900 mg).

LCMS: 236 [M+H]⁺.

### Intermediate 45

### 2-Chloro-7-methyl-N-(1-methyl-1H-imidazol-4-yl)-7H-pyrrolo[2,3-d]pyrimidin-4-amine, Trifluoroacetic Acid Salt

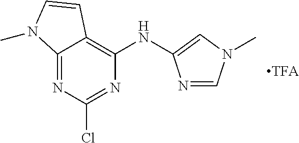

A mixture of 1-methyl-4-nitro-1H-imidazole (Intermediate 1, 1384 mg, 10.89 mmol) and Pd on charcoal (140 mg, 0.13 mmol) in ethanol (12 ml) was placed under H₂. After filtration through diatomaceous earth (Celite® brand), the filtrate was added to 2,4-dichloro-7-methyl-7H-pyrrolo[2,3-d]pyrimidine (Intermediate 46) and DIPEA (929 μA, 5.32 mmol) and the resulting mixture stirred at 90° C. for 15 hours. The reaction mixture was diluted with water and extracted with DCM/MeOH (10%). Evaporation of the volatiles under reduced pressure gave a residue, which was purified by reversed phase HPLC (Gilson® chromatography, MeCN/0.1% TFA in water 5%→45%) to provide the title product (300 mg).

LCMS: 265 [M+H]⁺.

### Intermediate 46

### 2,4-Dichloro-7-methyl-7H-pyrrolo[2,3-d]pyrimidine

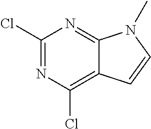

2,4-Dichloro-7H-pyrrolo[2,3-d]pyrimidine (2370 mg, 12.61 mmol) was dissolved in acetonitrile (8320 μl) and sodium hydride (529 mg, 13.24 mmol) was added portion-wise. The reaction mixture was stirred at room temperature for 30 minutes until gas evolution ceased. Methyl iodide (867 μl, 13.87 mmol) was added and the resulting mixture was stirred for 30 minutes. The reaction mixture was then poured into water and extracted with DCM/MeOH. Concentration of the organic layers under reduced pressure provided a residue, which was purified utilizing ISCO (0%→100% DCM/EtOAc) to afford the title product (2.1 g).

LCMS: 204 [M+H]⁺.

### Intermediate 47

### 2-Chloro-7-cyclopropyl-N-(1-methyl-1H-imidazol-4-yl)-7H-pyrrolo[2,3-d]pyrimidin-4-amine, Trifluoroacetic Acid Salt

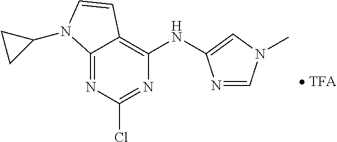

2,4-Dichloro-7-cyclopropyl-7H-pyrrolo[2,3-d]pyrimidine (Intermediate 48, 270 mg, 1.18 mmol) and 1-methyl-1H-imidazol-4-amine hydrochloride (Intermediate 36, 604 mg, 3.55 mmol) were reacted using a procedure similar to the one described for the synthesis of Intermediate 43, providing the title product (200 mg), after purification by reversed phase HPLC (Gilson® chromatography, MeCN/0.1% TFA in water 0%→50%).

LCMS: 291 [M+H]⁺.

### Intermediate 48

### 2,4-Dichloro-7-cyclopropyl-7H-pyrrolo[2,3-d]pyrimidine

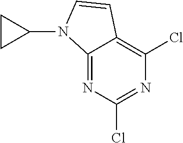

2,4-Dichloro-7H-pyrrolo[2,3-d]pyrimidine (1 g, 5.32 mmol), copper (II) acetate (1.449 g, 7.98 mmol), pyridine (2.151 ml, 26.59 mmol) and cyclopropylboronic acid (1.142 g, 13.30 mmol) were heated at 90° C. under dry air for 36 hours. The reaction mixture was concentrated under reduced pressure and the residue was partitioned between EtOAc and water. The organic layer was collected, dried and concentrated under reduced pressure to provide a crude mixture, which was purified utilizing ISCO (0%→30% Hexanes/EtOAc) to afford the title product (270 mg).

LCMS: 230 [M+H]⁺.

### Intermediate 49

### 2-Chloro-6-methoxy-N-(1-methyl-1H-imidazol-4-yl)quinazolin-4-amine

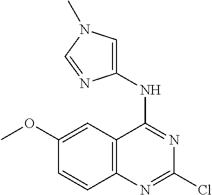

A mixture of 1-methyl-4-nitro-1H-imidazole (Intermediate 1, 1895 mg, 14.91 mmol) and Pd on charcoal (200 mg, 1.88 mmol) in ethanol (12.2 ml) was placed under H₂ for 3 hours. After filtration through diatomaceous earth (Celite® brand), the filtrate was added to a solution of 2,4-dichloro-6-methoxyquinazoline (Intermediate 50, 2277 mg, 9.94 mmol) in MeCN (12.2 ml) and DIPEA (8680 μl, 49.70 mmol) and the resulting mixture stirred at 70° C. overnight. The reaction mixture was diluted with water and extracted with DCM/MeOH (10%). The title product (710 mg) was collected after filtration as white fluffy solid. LCMS: 291 [M+H]⁺.

### Intermediate 50

### 2,4-Dichloro-6-methoxyquinazoline

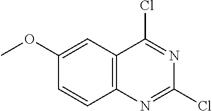

A solution of 6-methoxyquinazoline-2,4-diol (Intermediate 51, 1.91 g, 9.94 mmol) and N,N-dimethylaniline (1.260 ml, 9.94 mmol) in POCl₃ (13.90 ml, 149.09 mmol) was heated at reflux for 4 hours. The reaction mixture was cooled to rt and concentrated under reduced pressure to give the title product. The title product was used in the subsequent step without any further purification.

LCMS: 230 [M+H]⁺.

### Intermediate 51

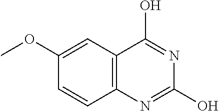

### 6-Methoxyquinazoline-2,4-diol

A mixture of 2-amino-5-methoxybenzoic acid (4 g, 23.93 mmol) and urea (5.89 g, 98.11 mmol) were pulverized and heated at 220° C. for 30 minutes. After cooling to room temperature, NaOH (38.3 ml, 38.29 mmol, 1N aq) was added. The mixture was heated until complete dissolution occurred and then was allowed to cool at ambient temperature before pouring it over solid CO₂. A white precipitate formed and the mixture was filtered, washed with cold water several times and dried to afford the title product (1.91 g).

LCMS: 192 [M+H]⁺.

### Intermediate 52

### 2-Chloro-7-methoxy-N-(1-methyl-1H-imidazol-4-yl)quinazolin-4-amine

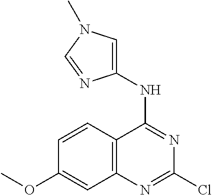

A mixture of 1-methyl-4-nitro-1H-imidazole (Intermediate 1, 1344 mg, 10.58 mmol) and Pd on charcoal (200 mg, 1.88 mmol) in ethanol (8672 μl) was placed under H₂ for 3 hours. After filtration through diatomaceous earth (Celite® brand), the filtrate was added to a solution of 2,4-dichloro-7-methoxyquinazoline (Intermediate 53,1615 mg, 7.05 mmol) in MeCN (8672 μl) and DIPEA (6157 μl, 35.25 mmol) and the resulting mixture stirred at 70° C. overnight. The reaction mixture was diluted with water and extracted with DCM/MeOH (10%). The title product (710 mg) was obtained after purification utilizing ISCO (0%→10% MeOH/DCM) as white solid.

LCMS: 291 [M+H]⁺.

### Intermediate 53

### 2,4-Dichloro-7-methoxyquinazoline

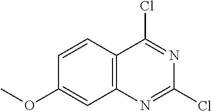

7-Methoxyquinazoline-2,4-diol (Intermediate 54, 1.35 g, 7.02 mmol), N,N-dimethylaniline (0.890 ml, 7.02 mmol) and POCl₃ (9.82 ml, 105.37 mmol) were reacted using a procedure similar to the one described for the synthesis of Intermediate 50, providing the title product which was used in the subsequent step without any further purification.

LCMS: 230 [M+H]⁺.

### Intermediate 54

### 7-Methoxyquinazoline-2,4-diol

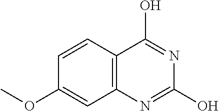

2-Amino-4-methoxybenzoic acid (5 g, 29.91 mmol) and urea (7.36 g, 122.64 mmol) were reacted using a procedure similar to the one described for the synthesis of Intermediate 51, providing the title product as brown solid (1.91 g).

LCMS: 192 [M+H]⁺.

### Intermediate 55

### 2-Chloro-6-fluoro-N-(1-methyl-1H-imidazol-4-yl)pyrido[2,3-d]pyrimidin-4-amine

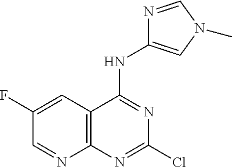

1-Methyl-4-nitro-1H-imidazole (Intermediate 1, 0.770 g, 6.05 mmol) and 2,4-dichloro-6-fluoropyrido[2,3-d]pyrimidine (Intermediate 56, 1.1 g, 5.05 mmol) were reacted using a procedure similar to the one described for the synthesis of Intermediate 52, providing the title product (1.010 g, 71.8%) as a yellow solid.

¹H NMR (300 MHz, DMSO-d6) δ ppm 3.82 (s, 3H) 7.49-7.81 (m, 2H) 9.06-9.39 (m, 2H)

LCMS: 279.0 [M+H]⁺.

### Intermediate 56

### 2,4-Dichloro-6-fluoropyrido[2,3-d]pyrimidine

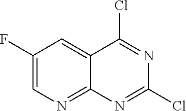

To a stirred suspension of the 6-fluoropyrido[2,3-d]pyrimidine-2,4-diol (Intermediate 57, 2.5 g, 13.80 mmol) in anhydrous toluene (28 mL) under an N₂ atmosphere was added slowly DIPEA (7.23 mL, 41.41 mmol). The reaction mixture was heated at 70° C. for 30 minutes and then cooled to room temperature prior to the addition of POCl₃ (3.86 mL, 41.41 mmol). The resulting reaction mixture was heated at 100° C. for 3 hours before being cooled and concentrated in vacuo to give a residue. Purification utilizing ISCO (25% Hexanes/EtOAc) gave the title product.

LCMS: 218.0 [M+H]⁺.

### Intermediate 57

### 6-Fluoropyrido[2,3-d]pyrimidine-2,4-diol

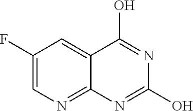

To a mixture of 2-amino-5-fluoronicotinic acid (Intermediate 58, 1.04 g, 6.66 mmol) and urea (1.640 g, 27.31 mmol) was pulverized and heated to 210° C. for 30 minutes. After cooling to room temperature, 2N NaOH (5.33 ml, 10.66 mmol) was added. The mixture was heated until complete dissolution occurred and was subsequently allowed to cool close to ambient temperature before pouring it over solid CO₂. A white precipitate formed, filtered and the white solid was washed with cold water (3×). The solid was suspended in glacial acetic acid (10 mL) and the mixture was heated at 100° C. for 1 h, cooled down and filtered to give the title product (0.368 g, 30.5%) as white solid. LCMS: 182.1 [M+H]⁺.

### Intermediate 58

### 2-Amino-5-fluoronicotinic acid

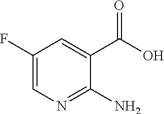

A solution of 2-chloro-5-fluoronicotinic acid (5 g, 28.48 mmol) in ((2,4-dimethoxyphenyl)methanamine (8.56 ml, 56.97 mmol) was heated at 100° C. overnight. The reaction mixture was concentrated under vacuum and partitioned between water and DCM. Evaporation of the organic layer provided a dark brown residue, which was dissolved in TFA (8.78 ml, 113.93 mmol) and the resulting mixture was stirred for 30 minutes. The precipitate formed was discarded via filtration and concentration of the filtrate under reduced pressure gave a residue. This residue was dissolved in HCl (1N, 200 mL) and the aqueous solution was washed with Et₂O and evaporated under reduced pressure to give a solid. This solid was washed with DCM/Hexanes, dried in a vacuum oven overnight and characterized as the title product (1.6 g).

LCMS: 156.0 [M+H]⁺.

¹H NMR (300 MHz, DMSO-d₆) δ ppm 8.23 (d, J=3.01 Hz, 1H), 7.86 (dd, J=8.95, 3.11 Hz, 1H)

### Intermediate 59

### 2-Chloro-N-(1-methyl-1H-imidazol-4-yl)-7-(trifluoromethyl)pyrido[2,3-d]pyrimidin-4-amine

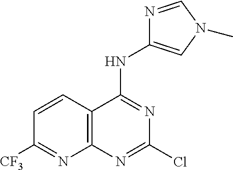

1-Methyl-4-nitro-1H-imidazole (Intermediate 1, 1.024 g, 8.06 mmol) and 2,4-dichloro-7-(trifluoromethyl)pyrido[2,3-d]pyrimidine (Intermediate 60, 1.8 g, 6.72 mmol) were reacted using a procedure similar to the one described for the synthesis of Intermediate 52, providing the title product (1.100 g, 49.8%), which was used in the next step without further purification.

LCMS: 329.0 [M+H]⁺.

### Intermediate 60

### 2,4-Dichloro-7-(trifluoromethyl)pyrido[2,3-d]pyrimidine

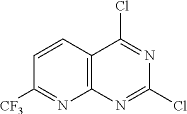

7-(Trifluoromethyl)pyrido[2,3-d]pyrimidine-2,4-diol (Intermediate 61, 1.66 g, 7.18 mmol) and POCl₃ (2.008 mL, 21.55 mmol) were reacted using a procedure similar to the one described for the synthesis of Intermediate 56, providing the title product (1.940 g) which was used in the next step without further purification.

LCMS: 268.0 [M+H]⁺.

### Intermediate 61

### 7-(Trifluoromethyl)pyrido[2,3-d]pyrimidine-2,4-diol

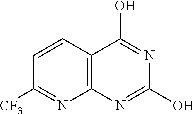

2-Amino-6-(trifluoromethyl)nicotinic acid (Intermediate 24, 1.9 g, 9.22 mmol) and urea (3.32 g, 55.31 mmol) were reacted using a procedure similar to the one described for the synthesis of Intermediate 57, providing the title product (1.660 g, 78%) as a white solid.

LCMS: 233.1 [M+H]⁺.

### Intermediate 62

### 2-Amino-6-chloronicotinic acid

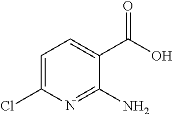

A solution of 2,6-dichloronicotinic acid (10 g, 52.08 mmol) and (2,4-dimethoxyphenyl)methanamine (15.65 ml, 104.17 mmol) in pyridine (21.06 ml, 260.42 mmol) was heated at 100° C. overnight. The reaction mixture was concentrated under vacuum and partitioned between water and DCM. Evaporation of the organic layer provided a dark brown residue, which was dissolved in TFA (8.78 ml, 113.93 mmol) and the resulting mixture was stirred for 30 minutes. The precipitate formed was discarded via filtration and concentration of the filtrate under reduced pressure gave a residue. This residue was dissolved in HCl (1N, 200 mL) and the aqueous solution was washed with Et₂O and evaporated under reduced pressure to give a solid. This solid was washed with DCM/Hexanes, dried in a vacuum oven overnight and characterized as the title product (6.8 g)

LCMS: 172.2 [M+H]⁺.

### Intermediate 63

### 2,7-Dichloro-N-(1-methyl-1H-imidazol-4-yl)pyrido[2,3-d]pyrimidin-4-amine

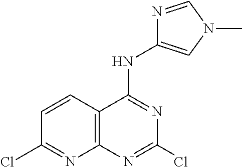

1-Methyl-4-nitro-1H-imidazole (Intermediate 1, 261 mg, 2.06 mmol) and 2,4,7-trichloropyrido[2,3-d]pyrimidine (Intermediate 64, 402 mg, 1.71 mmol) were reacted using a procedure similar to the one described for the synthesis of Intermediate 52, providing the title product (365 mg, 72.1%), which was used in next step without further purification. LCMS confirmed the target compound.

LCMS: 297.3 [M+H]⁺.

### Intermediate 64

### 2,4,7-Trichloropyrido[2,3-d]pyrimidine

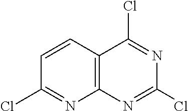

7-Chloropyrido[2,3-d]pyrimidine-2,4-diol (Intermediate 65, 1.744 g, 8.83 mmol) and POCl₃ (2.468 mL, 26.48 mmol) were reacted using a procedure similar to the one described for the synthesis of Intermediate 56, providing the title product after purification utilizing ISCO (1.030 g).

¹H NMR (300 MHz, CHLOROFORM-d) δ ppm 7.61 (d, J=8.67 Hz, 1H) 8.46 (d, J=8.67 Hz, 1H)

LCMS: 235.8 [M+H]⁺.

### Intermediate 65

### 7-Chloropyrido[2,3-d]pyrimidine-2,4-diol

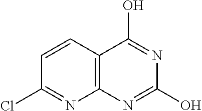

To a stirred solution (0.06 M) of 2-amino-6-chloronicotinamide (Intermediate 66, 1.86 g, 10.84 mmol) in anhydrous toluene (181 ml) under N₂ atmosphere was added oxalyl chloride (1.651 g, 13.01 mmol) in a drop-wise manner. The resulting mixture was heated at reflux (115° C.) for 4 hours whereupon it was cooled and stirred for a further 16 hours. The crude reaction mixture was concentrated to half its volume in vacuo and filtered to give the desired product (1.740 g, 81%) in suitably pure form to be used without any further purification

LCMS: 200.1 [M+H]⁺.

### Intermediate 66

### 2-Amino-6-chloronicotinamide

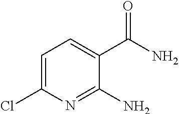

To a 0.3 M solution of 2-amino-6-chloronicotinic acid (Intermediate 62, 2.3 g, 13.33 mmol) in anhydrous THF (44 ml) under N₂ atmosphere, was added thionyl chloride (3.20 ml, 43.98 mmol) in a drop-wise manner. The reaction mixture was stirred at room temperature for 2 hours, whereupon it was concentrated in vacuo to give a yellow solid residue. The crude solid was dissolved in THF (44 ml) and the volatiles were removed under reduced pressure (this process was repeated twice). Finally the yellow solid was re-dissolved in THF (44 ml) and ammonia gas bubbled through the solution for 1 hour. The resulting precipitate was removed by filtration and the filtrate was concentrated in vacuo to give a yellow precipitate which was triturated with water at 50° C., dried and characterized as the title product (1.860 g, 81%).

### Intermediate 67

### 2-Chloro-N-(1-methyl-1H-imidazol-4-yl)pyrido[3,4-d]pyrimidin-4-amine

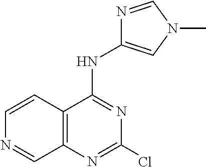

1-Methyl-4-nitro-1H-imidazole (Intermediate 1, 381 mg, 3.00 mmol) and 2,4-dichloropyrido[3,4-d]pyrimidine (500 mg, 2.50 mmol) were reacted using a procedure similar to the one described for the synthesis of Intermediate 52, providing the title product.

LCMS: 261.0 [M+H]⁺.

### Intermediate 68

### 2-Chloro-N-(1-methyl-1H-imidazol-4-yl)quinazolin-4-amine

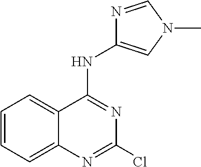

1-Methyl-4-nitro-1H-imidazole (Intermediate 1, 429 mg, 3.38 mmol) and 2,4-dichloroquinazoline (560 mg, 2.81 mmol) were reacted using a procedure similar to the one described for the synthesis of Intermediate 52, providing the title product after purification utilizing ISCO (5%→10% MeOH/DCM) (530 mg).

### Intermediate 69

### 6-Chloro-N-(1-methyl-1H-imidazol-4-yl)-1-(tetrahydro-2H-pyran-2-yl)-1H-pyrazolo[3,4-d]pyrimidin-4-amine

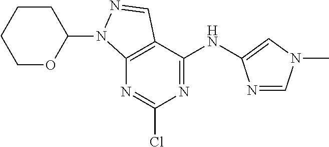

To a solution of 4,6-dichloro-1-(tetrahydro-2H-pyran-2-yl)-1H-pyrazolo[3,4-d]pyrimidine (Intermediate 70, 3.168 g, 11.60 mmol) in ethanol (60 mL), was added TEA (4.04 mL, 29.00 mmol) followed by 1-methyl-1H-imidazol-4-amine hydrochloride (Intermediate 36, 1.549 g, 11.60 mmol). The resulting mixture was heated at 60° C. for 2 hours. Evaporation of the volatiles under reduced pressure gave a residue, which was purified utilizing ISCO (EtOAc/hexanes 0→80%) to give the title product (1.56 g).

LCMS: 334 [M+H]⁺.

### Intermediate 70

### 4,6-Dichloro-1-(tetrahydro-2H-pyran-2-yl)-1H-pyrazolo[3,4-d]pyrimidine

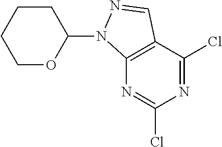

To a solution of 4,6-dichloro-1H-pyrazolo[3,4-d]pyrimidine (Intermediate 71, 2 g, 10.58 mmol) and p-Ts-OH (0.201 g, 1.06 mmol) in DCM (30 mL) and THF (30.0 mL), was added 3,4-dihydro-2H-pyran (1.335 g, 15.87 mmol). The resulting solution was stirred overnight at ambient temperature whereupon the volatiles were removed under reduced pressure. The residue left, was dissolved in DCM and the organic layer was washed with saturated aqueous sodium carbonate solution, water, brine and dried (MgSO₄). Evaporation of the volatiles under reduced pressure gave the title product (2.80 g).

LCMS: 273[M+H]⁺.

### Intermediate 71

### 4,6-Dichloro-1H-pyrazolo[3,4-d]pyrimidine

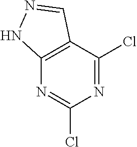

1H-Pyrazolo[3,4-d]pyrimidine-4,6(5H,7H)-dione (10 g, 65.74 mmol) was added slowly to a mixture of phosphorus oxychloride (60 ml, 643.70 mmol) and N,N-dimethylaniline (20 mL, 138.06 mmol). The resulting solution was heated at 110° C. for 2 hours whereupon the excess POCl₃ was evaporated. The crude mixture was poured onto crushed ice (100 mL), and the aqueous layer was extracted with ether (300 mL×3). The combined organic extracts were dried (MgSO₄), filtered, and evaporated in vacuo to afford the title product (9.14 g).

### Example 1

### N²-[(1S)-1-(5-Fluoropyrimidin-2-yl)ethyl]-N⁴-(1-methyl-1H-imidazol-4-yl)thieno[2,3-d]pyrimidine-2,4-diamine, Trifluoroacetic Acid Salt

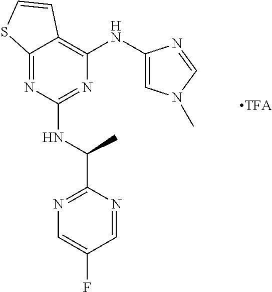

A microwave tube was charged with 2-chloro-N-(1-methyl-1H-imidazol-4-yl)thieno[2,3-d]pyrimidin-4-amine (Intermediate 7, 287 mg, 1.08 mmol) and (1S)-1-(5-fluoropyrimidin-2-yl)ethanamine hydrochloride (Intermediate 6, 192 mg, 1.08 mmol), n-BuOH (5 mL) and triethylamine (0.376 mL, 2.70 mmol). The reaction mixture was heated in a microwave at 160° C. for 3 hours. Evaporation of the volatiles under reduced pressure gave a residue. The residue was purified by reversed-phase HPLC (Gilson® chromatography, 2%→59% MeCN/H₂O (0.1% TFA), 35 min, Xterra Prep, 100 mg/mL, 3.0 mL inj, 254 nm). Concentration of the fractions in vacuo provided the title product as part of a mixture of enantiomers (155 mg) in the form of a yellow solid, the title enantiomer being present in the mixture in an amount greater than or equal to the amount of the corresponding R enantiomer.

¹H NMR (300 MHz, MeOD) δ ppm 8.77 (s, 2H) 8.20 (d, 1H) 7.97 (bs, 1H) 7.52 (bs, 1H) 7.29 (d, 1H) 5.43 (q, 1H) 3.90 (s, 3H) 1.72 (d, 3H).

LCMS: 371 [M+H]⁺.

### Column and Solvent Conditions

The R and S enantiomers were separated using chiral HPLC (Chiralpak® AD column).

Column dimensions: 2×25 cm, 10μ
Mobile phase: 50:50:0.1 Hexane:Isopropanol:diethylamine
Flow rate (ml/min): 20 mL/min

### Detection (nm): 220 nm

### Post Purification Purity Check

Sample purity was checked with an AD-H column.

Column dimensions: 4.6×250 mm, 10μ,
Mobile phase: 50:50:0.1 Hexane:Isopropanol:diethylamine
Flow: 1.0 mL/min

### Detection: 220 nm

### Example 1(a)

### First Eluting Compound

### N²-[1-(5-Fluoropyrimidin-2-yl)ethyl]-N⁴-(1-methyl-1H-imidazol-4-yl)thieno[2,3-d]pyrimidine-2,4-diamine, Enantiomer (A)

The first eluting compound had a retention time of 9.36 minutes, >98% ee.

¹H NMR (300 MHz, MeOD) δ ppm 8.72 (s, 2H) 7.83 (d, 1H) 7.47 (bs, 2H) 7.07 (d, 1H) 5.41 (q, 1H) 3.82 (s, 3H) 1.64 (d, 3H).

LCMS: 371 [M+H]⁺.

### Example 1(b)

### Second Eluting Compound

### N²-[1-(5-Fluoropyrimidin-2-yl)ethyl]-N⁴-(1-methyl-1H-imidazol-4-yl)thieno[2,3-d]pyrimidine-2,4-diamine, Enantiomer (B)

The second eluting compound had a retention time of 23.82 minutes, >98% ee.

¹H NMR (300 MHz, MeOD) δ ppm 8.71 (s, 2H) 7.80 (d, 1H) 7.48 (s, 1H) 7.46 (s, 1H) 7.05 (d, 1H) 5.41 (q, 1H) 3.82 (s, 3H) 1.63 (d, 3H).

LCMS: 371 [M+H]⁺.

### Example 2

### N²-[(1S)-1-(5-Fluoropyrimidin-2-yl)ethyl]-7-methyl-N⁴-(1-methyl-1H-imidazol-4-yl)thieno[3,2-d]pyrimidine-2,4-diamine, Trifluoroacetic Acid Salt

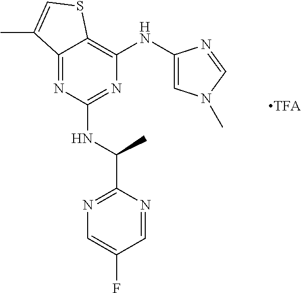

2-Chloro-7-methyl-N-(1-methyl-1H-imidazol-4-yl)thieno[3,2-d]pyrimidin-4-amine (Intermediate 8, 276 mg, 0.99 mmol) and (1S)-1-(5-fluoropyrimidin-2-yl)ethanamine hydrochloride (Intermediate 6, 175 mg, 0.99 mmol) were reacted using a procedure similar to the one described for the synthesis of Example 1, providing the title product as part of a mixture of enantiomers (126 mg) in the form of a yellow solid, the title enantiomer being present in the mixture in an amount greater than or equal to the amount of the corresponding R enantiomer.

¹H NMR (300 MHz, MeOD) δ ppm 8.79 (s, 2H) 7.97 (bs, 1H) 7.86 (s, 1H) 7.52 (s, 1H) 5.46 (q, 1H) 3.91 (s, 3H) 2.40 (s, 3H) 1.70 (d, 3H).

LCMS: 385 [M+H]⁺

### Column and Solvent Conditions

The R and S enantiomers of the title product were separated using chiral HPLC (Chiralpak® AD column).

Column dimensions: 2×25 cm, 10μ
Mobile phase: 50:50:0.1 Hexane:Isopropanol:diethylamine
Flow rate (ml/min): 20 mL/min

### Detection (nm): 220 nm

### Post Purification Purity Check

Sample purity was checked with a AD-H column.

Column dimensions: 4.6×250 mm, 10μ
Mobile phase: 50:50:0.1 Hexane:Isopropanol:diethylamine
Flow: 1.0 mL/min

### Detection: 220 nm

### Example 2(a)

### First Eluting Compound

### N²-[1-(5-Fluoropyrimidin-2-yl)ethyl]-7-methyl-N⁴-(1-methyl-1H-imidazol-4-yl)thieno[3,2-d]pyrimidine-2,4-diamine, Enantiomer (A)

The first eluting compound had a retention time of 8.38 minutes, >98% ee.

¹H NMR (300 MHz, MeOD) δ ppm 8.71 (s, 2H) 7.47-7.41 (m, 3H) 5.42 (q, 1H) 3.82 (s, 3H) 2.26 (s, 3H) 1.63 (d, 3H).

LCMS: 385 [M+H]⁺.

### Example 2(b)

### Second Eluting Compound

### N²-[1-(5-Fluoropyrimidin-2-yl)ethyl]-7-methyl-N⁴-(1-methyl-1H-imidazol-4-yl)thienol[3,2-d]pyrimidine-2,4-diamine, Enantiomer (B)

The second eluting compound had a retention time of 15.82 minutes, >98% ee.

¹H NMR (300 MHz, MeOD) δ ppm 8.71 (s, 2H) 7.47-7.41 (m, 3H) 5.42 (q, 1H) 3.82 (s, 3H) 2.26 (s, 3H) 1.63 (d, 3H).

LCMS: 385 [M+H]⁺.

### Example 3

### N²-[(1S)-1-(5-Fluoropyrimidin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine

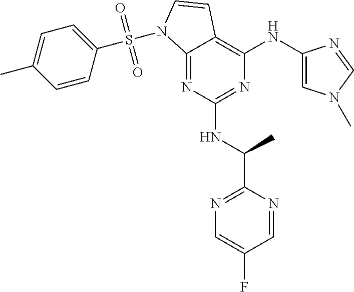

In a microwave tube, 2-chloro-N-(1-methyl-1H-imidazol-4-yl)-7-[(4-methylphenyl)sulfonyl]-7H-pyrrolo[2,3-d]pyrimidin-4-amine (Intermediate 10, 90 mg), (1S)-1-(5-fluoropyrimidin-2-yl)ethanamine hydrochloride (Intermediate 6, 881 mg, 4.96 mmol) and DIPEA (1.084 mL, 6.21 mmol) were dissolved in n-BuOH (5 mL). The reaction mixture was heated in a microwave reactor at 180° C. for 3 hr. After completion of the reaction as indicated by LCMS, the reaction mixture was evaporated in vacuo to obtain a brown residue, which was purified by column chromatography (4% MeOH, 0.4% NH₄OH in DCM) to provide the title product as part of a mixture of enantiomers (350 mg, 56%) in the form of a yellow solid, the title enantiomer being present in the mixture in an amount greater than or equal to the amount of the corresponding R enantiomer.

LCMS: 508 [M+H]⁺.

The title product was also synthesized by the following procedure:

A mixture of (1S)-1-(5-fluoropyrimidin-2-yl)ethanamine hydrochloride (Intermediate 6, 5290 mg, 29.79 mmol), 2-chloro-N-(1-methyl-1H-imidazol-4-yl)-7-[(4-methylphenyl)sulfonyl]-7H-pyrrolo[2,3-d]pyrimidin-4-amine (Intermediate 10, 6000 mg, 14.89 mmol), palladium(II) acetate (334 mg, 1.49 mmol), (R)-(−)-1-[(S)-2-(dicyclohexylphosphino) ferrocenyl]ethyldi-t-butylphosphine] (1302 mg, 2.38 mmol) and CS₂CO₃ (1940 mg, 59.57 mmol) in DMA (99 mL) was stirred at room temperature for 10 minutes under vacuum. The reaction flask was back filled with nitrogen was subsequently heated at 90° C. overnight. The reaction mixture was diluted with DCM/MeOH (10%) and the organic layer was washed with water. Concentration of the organic layer under reduced pressure provided residue, which was purified utilizing ISCO (0%→20% MeOH/DCM) to provide the title product as part of a mixture of enantiomers, the title enantiomer being present in the mixture in an amount greater than or equal to the amount of the corresponding R enantiomer.

LCMS: 506 [M+H]⁺.

### Example 4

### N²-[(1S)-1-(5-Fluoropyrimidin-2-yl)ethyl]-N⁴-(1-methyl-1H-imidazol-4-yl)-7H-pyrrolo[2,3-d]pyrimidine-2,4-diamine

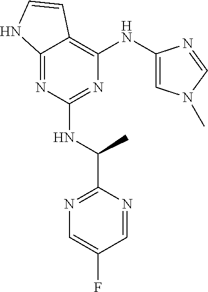

N²-[(1S)-1-(5-Fluoropyrimidin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine (Example 3, 270 mg, 0.53 mmol) and Cs₂CO₃ (520 mg, 1.60 mmol) were dissolved in MeOH (1.0 mL) and THF (1.0 mL). The reaction mixture was heated at 50° C. for 5 hr. The mixture was diluted with DCM and H₂O and separated. The organic layer was collected and washed with brine and dried over Na₂SO₄ and concentrated in vacuo to give a residue. The residue was purified by ISCO chromatography (4% MeOH, 0.4% NH₄OH in DCM) to provide the title product as part of a mixture of enantiomers (45 mg) in the form of a brown solid, the title enantiomer being present in the mixture in an amount greater than or equal to the amount of the corresponding R enantiomer.

The title product was also synthesized by the following procedure:

A solution of N²-[(1S)-1-(5-fluoropyrimidin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine (Example 3, 7613 mg, 15 mmol) and KOH (16.8 g, 300.00 mmol) in water (10 mL), methanol (10 mL) and 1,4-dioxane (52 mL) was heated at 55° C. overnight. The reaction mixture was acidified with HCl to pH=3 and washed with DCM. The aq. layer was neutralized with NaHCO₃ to pH8 and extracted with DCM/MeOH (10%). The organic layer was concentrated under reduced pressure to give a residue. The residue was purified utilizing ISCO (0%→80% DCM/Acetone/2% NH₄OH) to provide the title product as part of a mixture of enantiomers, the title enantiomer being present in the mixture in an amount greater than or equal to the amount of the corresponding R enantiomer.

¹H NMR (300 MHz, MeOD) δ ppm 8.68 (s, 2H), 7.47 (d, 1H), 7.39 (d, 1H), 6.74 (d, 1H), 6.37 (d, 1H), 5.39 (q, 1H), 3.80 (s, 3H), 1.59 (d, 4H).

LCMS: 354 [M+H]⁺.

### Column and Solvent Conditions

The R and S enantiomers were separated using chiral HPLC (Chiralpak® AD column).

Column dimensions: 50×500 mm, 20μ
Mobile phase: 1:1:0.1% Methanol:EthanoL:diethylamine
Flow rate (ml/min): 120

### Detection (nm): 220

### Post Purification Purity Check

Sample purity was checked with Chiralpak® AD.

Column dimensions: 4.6×100 mm, 5μ
Mobile phase: 60%:40%:0.4% CarbonDioxide:Methanol:diethylamine
Flow: 5.0 mL/min

### Detection: 220 nm

### Example 4(a)

### First Eluting Compound

### N²-[1-(5-Fluoropyrimidin-2-yl)ethyl]-N⁴-(1-methyl-1H-imidazol-4-yl)-7H-pyrrolo[2,3-d]pyrimidine-2,4-diamine, Enantiomer (A)

The first eluting compound had a retention time of 1.64 minutes, >98% ee.

¹H NMR (300 MHz, MeOD) δ ppm 8.68 (s, 2H), 7.47 (d, J=1.51 Hz, 1H), 7.39 (d, J=1.13 Hz, 1H), 6.74 (d, J=3.58 Hz, 1H), 6.37 (d, J=3.58 Hz, 1H), 5.39 (q, J=7.03 Hz, 1H), 3.80 (s, 3H), 1.59 (d, J=6.97 Hz, 4H).

LCMS: 354 [M+H]⁺.

### Example 4(b)

### Second Eluting Compound

### N²-[1-(5-Fluoropyrimidin-2-yl)ethyl]-N⁴-(1-methyl-1H-imidazol-4-yl)-7H-pyrrolo[2,3-d]pyrimidine-2,4-diamine, Enantiomer (B)

The second eluting compound had a retention time of 3.21 minutes, >98% ee.

¹H NMR (300 MHz, MeOD) δ ppm 8.69 (s, 2H), 7.48 (d, J=1.51 Hz, 1H), 7.39 (d, J=1.13 Hz, 1H), 6.74 (d, J=3.58 Hz, 1H), 6.37 (d, J=3.58 Hz, 1H), 5.39 (q, J=7.03 Hz, 1H), 3.80 (s, 3H), 1.59 (d, J=6.97 Hz, 4H).

LCMS: 354 [M+H]⁺.

### Example 5

### N²-[(1S)-1-(5-Fluoropyrimidin-2-yl)ethyl]-N⁴-(1-methyl-1H-imidazol-4-yl)-5-[(4-methylphenyl)sulfonyl]-5H-pyrrolo[3,2-d]pyrimidine-2,4-diamine

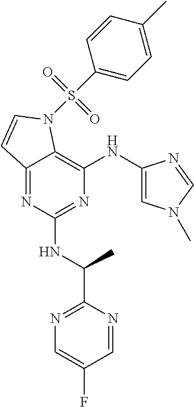

2-Chloro-N-(1-methyl-1H-imidazol-4-yl)-5-[(4-methylphenyl)sulfonyl]-5H-pyrrolo[3,2-d]pyrimidin-4-amine (Intermediate 12, 65 mg, 0.16 mmol) and (1S)-1-(5-fluoropyrimidin-2-yl)ethanamine hydrochloride (Intermediate 6, 114 mg, 0.64 mmol) were reacted using a procedure similar to the one described for the synthesis of Example 3, providing the title product as part of a mixture of enantiomers (25 mg), the title enantiomer being present in the mixture in an amount greater than or equal to the amount of the corresponding R enantiomer.

¹H NMR (400 MHz, MeOD) δ ppm 8.60 (s, 1H) 8.54 (s, 2H) 7.62 (d, J=3.54 Hz, 1H) 7.54 (d, J=8.34 Hz, 2H) 7.34 (s, 1H) 7.14 (d, J=8.08 Hz, 2H) 6.33 (d, J=3.79 Hz, 1H) 5.21 (q, J=7.07 Hz, 1H) 3.69 (s, 3H) 2.18 (s, 3H) 1.46 (d, J=7.07 Hz, 3H).

LCMS: 508 [M+H]⁺.

### Example 6

### N²-[(1S)-1-(5-Fluoropyrimidin-2-yl)ethyl]-N⁴-(1-methyl-1H-imidazol-4-yl)-5H-pyrrolo[3,2-d]pyrimidine-2,4-diamine

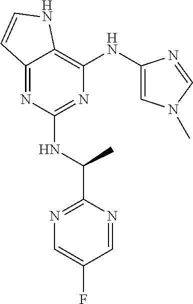

N²-[1-(5-Fluoropyrimidin-2-yl)ethyl]-N⁴-(1-methyl-1H-imidazol-4-yl)-5-[(4-methylphenyl)sulfonyl]-5H-pyrrolo[3,2-d]pyrimidine-2,4-diamine (Example 5, 25 mg, 0.05 mmol) was reacted using a procedure similar to the one described for the synthesis of Example 4, providing the title product as part of a mixture of enantiomers (13 mg), the title enantiomer being present in the mixture in an amount greater than or equal to the amount of the corresponding R enantiomer.

¹H NMR (400 MHz, MeOD) δ ppm 8.59 (s, 2H) 7.39 (s, 1H) 7.31 (s, 1H) 7.21 (d, J=3.03 Hz, 1H) 6.09 (d, J=2.78 Hz, 1H) 5.29 (q, J=6.91 Hz, 1H) 3.70 (s, 3H) 1.52 (d, J=6.82 Hz, 3H).

LCMS: 354 [M+H]⁺.

### Example 7

### N⁵-[(1S)-1-(5-Fluoropyrimidin-2-yl)ethyl]-2-methyl-N⁷-(1-methyl-1H-imidazol-4-yl)[1,3]thiazolo[5,4-d]pyrimidine-5,7-diamine, Trifluoroacetic Acid Salt

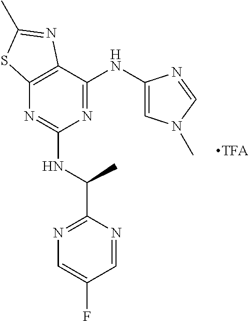

To a mixture of 5-chloro-2-methyl-N-(1-methyl-1H-imidazol-4-yl)[1,3]thiazolo[5,4-d]pyrimidin-7-amine (Intermediate 13, 250 mg, 0.87 mmol) and (1S)-1-(5-fluoropyrimidin-2-yl)ethanamine hydrochloride (Intermediate 6) in n-BuOH (2 mL), was added DIPEA. The mixture was heated overnight at 70° C. Evaporation of the volatiles under reduced pressure gave a residue that was purified by reversed phase HPLC (Gilson® chromatography, 5%→65% MeCN/0.1% TFA in H₂O) to give the title product as part of a mixture of enantiomers (140 mg, 47.5%), the title enantiomer being present in the mixture in an amount greater than or equal to the amount of the corresponding R enantiomer.

¹H NMR (300 MHz, MeOD) δ ppm 8.61 (s, 2H), 7.43 (d, J=1.70 Hz, 1H), 5.21 (q, J=6.97 Hz, 1H), 3.88 (s, 3H), 2.60 (s, 3H), 1.52 (d, J=6.97 Hz, 3H).

LCMS: 386 [M+H]⁺.

### Column and Solvent Conditions

The R and S enantiomers were separated using Chiral SFC, (Chiralcel OD-H column).

Column dimensions: 21×250 mm, 5μ
Modifier: 30% Methanol with 0.4% Dimethylethylamine
Flow: 60 mL/min

### Outlet Pressure: 100 ba

### Column Temp: 40° C.

### Wavelength: 254

### Post Purification Purity Check

Sample purity was checked by SFC with an OD-H column.

Column dimensions: 4.6×100 mm
Modifier: 30% Methanol with 0.4% dimethylethylamine
Flow: 5 mL/min

### Outlet Pressure: 120 bar

### Detection: 254 nm

### Example 7(a)

### First Eluting Compound

### N⁵-[1-(5-Fluoropyrimidin-2-yl)ethyl]-2-methyl-N⁷-(1-methyl-1H-imidazol-4-yl)[1,3]thiazolo[5,4-d]pyrimidine-5,7-diamine, Enantiomer (A)

The first eluting compound had a retention time of 1.63 minutes, >98% ee.

¹H NMR (300 MHz, MeOD) δ ppm 8.61 (s, 2H), 7.42 (m, 2H), 5.25 (q, J=6.91 Hz, 1H), 3.71 (s, 3H), 2.58 (s, 3H), 1.51 (d, J=6.97 Hz, 3H)

LCMS: 386 [M+H]⁺.

### Example 7(b)

### Second Eluting Compound

### N⁵-[1-(5-Fluoropyrimidin-2-yl)ethyl]-2-methyl-N⁷-(1-meth 1-1H-imidazol-4-yl)[1,3]thiazolo[5,4-d]pyrimidine-5,7-diamine, Enantiomer (B)

The second eluting compound had a retention time of 2.39 minutes, >96.8% ee.

¹H NMR (300 MHz, MeOD) δ ppm 8.60 (s, 2H), 7.47 (m, 2H), 5.24 (d, J=7.16 Hz, 1H), 3.72 (s, 3H), 2.58 (s, 3H), 1.51 (d, J=6.97 Hz, 3H).

LCMS: 386 [M+H]⁺.

### Example 8

### N²-[(1S)-1-(5-Fluoropyrimidin-2-yl)ethyl]-N⁴-(1-methyl-1H-imidazol-4-yl)-6,7-dihydro-5H-cyclopenta[d]pyrimidine-2,4-diamine, Trifluoroacetic Acid Salt

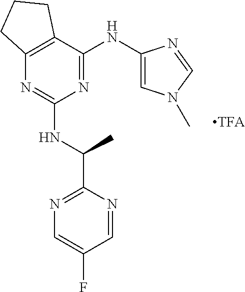

To a suspension of 2-chloro-N-(1-methyl-1H-imidazol-4-yl)-6,7-dihydro-5H-cyclopenta[d]pyrimidin-4-amine (Intermediate 14, 350 mg, 1.4 mmol) and (1S)-1-(5-fluoropyrimidin-2-yl)ethanamine hydrochloride (Intermediate 6, 298 mg, 1.68 mmol) in n-BuOH (2 mL), DIPEA (0.734 mL, 4.21 mmol) was added and the mixture was heated to 150° C. in a microwave for 6 hours. The volatiles were evaporated under reduced pressure to give a residue. Purification by reversed phase (Gilson® chromatography, 5%→50% MeCN/0.1% TFA in H₂O) gave the title product as part of a mixture of enantiomers (310 mg, 47.2%), the title enantiomer being present in the mixture in an amount greater than or equal to the amount of the corresponding R enantiomer.

¹H NMR (300 MHz, MeOD) δ ppm 8.64 (s, 2H), 8.55 (s, 1H), 7.50 (br. s., 1H), 5.21 (q, J=6.78 Hz, 1H), 3.89 (s, 3H), 2.90 (t, J=7.72 Hz, 2H), 2.73 (t, J=7.35 Hz, 2H), 2.15 (quin, J=7.54 Hz, 2H), 1.56 (d, J=6.97 Hz, 3H).

LCMS: 355 [M+H]⁺.

### Column and Solvent Conditions

The R and S enantiomers were separated using chiral HPLC (Chiralpak® AD).

Column dimensions: 50×500 mm, 20μ
Mobile phase B: 1:1 Methanol:Ethanol, Additive: 0.1% diethylamine
Flow rate (ml/min): 120 mL/min

### Detection (nm): 220

### Post Purification Purity Check

Sample purity was checked by chiral HPLC

### Column: Chiralpak® AD

Column dimensions: 4.6×250 mm, 10μ
Mobile phase B: 1:1 Methanol:Ethanol, Additive: 0.4% diethylamine
Flow rate (ml/min): 1 mL/min

### Detection (nm): 254

### Example 8(a)

### First Eluting Compound

### N²-[1-(5-Fluoropyrimidin-2-yl)ethyl]-N⁴-(1-methyl-1H-imidazol-4-yl)-6,7-dihydro-5H-cyclopenta[d]pyrimidine-2,4-diamine, Enantiomer (A)

The first eluting compound had a retention time of 2.69 min, >98% ee

¹H NMR (300 MHz, MeOD) δ ppm 8.59 (s, 2H), 7.25 (m, 2H), 5.22 (m, 0H), 5.23 (q, J=6.97 Hz, 1H), 3.67 (s, 3H), 2.59 (m, 4H), 1.96 (quin, J=7.54 Hz, 2H), 1.48 (d, J=7.16 Hz, 3H)

LCMS: 355.1 [M+H]⁺

### Example 8(b)

### Second Eluting Compound

### N²-[1-(5-Fluoropyrimidin-2-yl)ethyl]-N⁴-(1-methyl-1H-imidazol-4-yl)-6,7-dihydro-5H-cyclopenta[d]pyrimidine-2,4-diamine, Enantiomer (B)

The second eluting compound had a retention time of 3.86 min, >98% ee

¹H NMR (300 MHz, MeOD) δ ppm 8.58 (s, 2H), 7.24 (m, 2H), 5.23 (q, J=6.91 Hz, 1H), 3.67 (s, 3H), 2.59 (m, 4H), 1.96 (quin, J=7.49 Hz, 2H), 1.48 (d, J=6.97 Hz, 3H).

LCMS: 355.1 [M+H]⁺.

### Example 9

### 1-Ethyl-N⁶-[(1S)-1-(5-fluoropyrimidin-2-yl)ethyl]-N⁴-(1-methyl-1H-imidazol-4-yl)-1H-pyrazolo[3,4-d]pyrimidine-4,6-diamine, Trifluoroacetic Acid Salt

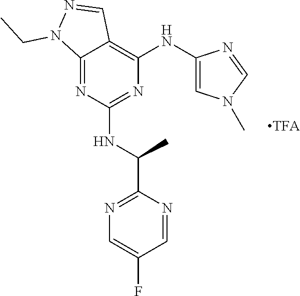

1-Ethyl-N-(1-methyl-1H-imidazol-4-yl)-6-(methylsulfonyl)-1H-pyrazolo[3,4-d]pyrimidin-4-amine (Intermediate 19, 190 mg, 0.59 mmol) and (1S)-1-(5-fluoropyrimidin-2-yl)ethanamine hydrochloride (Intermediate 6, 126 mg, 0.71 mmol) were dissolved in NMP (2 mL) and TEA (0.330 mL, 2.36 mmol) was added. The reaction was heated at 160° C. overnight. The reaction mixture was separated between EtOAC and water, washed with brine and dried with MgSO₄. Concentration in vacuo gave a brown oil (543 mg). Purification by reversed phase HPLC (Gilson® chromatography, using Atlantis Prep T3 column, 19×100 mm, 100 mg/mL, 400 μL inj, 15-34% MeCN/Water/0.1% TFA, elution time: 8 min, detection 240 nm). Concentration of the fractions in vacuo provided the title product as part of a mixture of enantiomers (33 mg) in the form of a solid brown residue, the title enantiomer being present in the mixture in an amount greater than or equal to the amount of the corresponding R enantiomer.

¹H NMR (300 MHz, MeOD) δ ppm 8.71 (s, 2H) 8.47 (br. s., 1H) 7.90 (s, 1H) 7.37 (s, 1H) 5.37 (q, 1H) 4.16 (q, 2H) 3.96 (s, 3H) 1.64 (d, 3H) 1.30 (t, 3H).

LCMS: 383 [M+H]⁺.

### Column and Solvent Conditions

The R and S enantiomers were separated using Chiral SFC, (Chiralpak® AD column).

Column dimensions: 21×250 mm, 5μ
Modifier: 40% Methanol with 0.4% Dimethylethylamine
Flow rate (ml/min): 60
Outlet Pressure (bar): 100

### Detection (nm): 220

### Post Purification Purity Check

Sample purity was checked by SFC with a AD-H column.

Column dimensions: 4.6×100 mm
Modifier: 40% Methanol with 0.4% Dimethylethylamine
Flow: 5 mL/min

### Outlet Pressure: 120 bar

### Detection: 254 nm

### Example 9(a)

### First Eluting Compound

### 1-Ethyl-N⁶-[1-(5-fluoropyrimidin-2-yl)ethyl]-N⁴-(1-methyl-1H-imidazol-4-yl)-1H-pyrazolo[3,4-d]pyrimidine-4,6-diamine, Enantiomer (A)

The first eluting compound had a retention time of 1.13 minutes, >98% ee.

¹H NMR (300 MHz, MeOD) δ ppm 8.69 (s, 2H) 7.84 (br. s., 1H) 7.55 (br. s., 1H) 7.43 (br. s., 1H) 5.39 (q, 1H) 3.96-4.33 (m, 2H) 3.79 (s, 3H) 1.61 (d, 3H) 1.19-1.49 (m, 3H).

LCMS: 383 [M+H]⁺.

### Example 9(b)

### Second Eluting Compound

### 1-Ethyl-N⁶-[1-(5-fluoropyrimidin-2-yl)ethyl]-N⁴-(1-methyl-1H-imidazol-4-yl)-1H-pyrazolo[3,4-d]pyrimidine-4,6-diamine, Enantiomer (B)

The second eluting compound had a retention time of 1.88 minutes, >98% ee.

¹H NMR (300 MHz, MeOD) δ ppm 8.69 (s, 2H) 7.82 (br. s., 1H) 7.54 (br. s., 1H) 7.43 (br. s., 1H) 5.39 (q, 1H) 4.16 (q, 2H) 3.78 (s, 3H) 1.60 (d, 3H) 1.31 (t, 3H).

LCMS: 383 [M+H]⁺.

### Example 10

### N²-[(1S)-1-(5-Fluoropyrimidin-2-yl)ethyl]-N⁴-(1-methyl-1H-imidazol-4-yl)pteridine-2,4-diamine

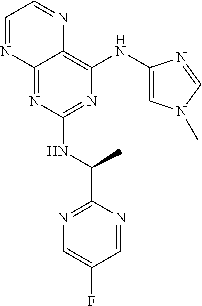

2-Chloro-N-(1-methyl-1H-imidazol-4-yl)pteridin-4-amine (Intermediate 21, 135 mg, 0.52 mmol) and (1S)-1-(5-fluoropyrimidin-2-yl)ethanamine hydrochloride (Intermediate 6, 183 mg, 1.03 mmol) were suspended in butan-1-ol (2 mL) and DIPEA (0.360 mL, 2.06 mmol) was added. The reaction was irradiated in a microwave at 160° C. for 36000 s. The reaction mixture was concentrated in vacuo leaving an amber oil (423 mg). This material was purified by ISCO (2-10% MeOH/DCM). Concentration of the fractions in vacuo provided the title product as part of a mixture of enantiomers (72 mg) in the form of a yellow solid, the title enantiomer being present in the mixture in an amount greater than or equal to the amount of the corresponding R enantiomer.

¹H NMR (300 MHz, MeOD) δ ppm 8.67-8.80 (m, 2.36H) 8.61 (br. s., 0.56H) 8.36 (br. s., 1H) 7.79 (br. s., 0.43H) 7.55 (br. s., 0.37H) 7.43 (d, 1H) 5.30-5.72 (m, 1H) 3.59-4.04 (m, 3H) 1.49-1.81 (m, 3H).

LCMS: 367 [M+H]⁺.

### Column and Solvent Conditions

The R and S enantiomers were separated using Chiral SFC, (Chiralpak® AD column).

Column dimensions: 21×250 mm, 5μ
Modifier: 20% Methanol with 0.4% Dimethylethylamine
Flow rate (ml/min): 40
Outlet Pressure (bar): 100

### Detection (nm): 254

### Post Purification Purity Check

Sample purity was checked by SFC with a AD column.

Column dimensions: 4.6×250 mm
Modifier: 20% Methanol with 0.4% Dimethylethylamine
Flow: 2.5 mL/min

### Outlet Pressure: 120 bar

### Detection: 254 nm

### Example 10(a)

### First Eluting Compound

### N²-[1-(5-Fluoropyrimidin-2-yl)ethyl]-N⁴-(1-methyl-1H-imidazol-4-yl)pteridine-2,4-diamine, Enantiomer (A)

The first eluting compound had a retention time of 11.21 minutes, 97.7% ee.

¹H NMR (300 MHz, MeOD) δ ppm 8.55-8.87 (m, 3H) 8.37 (d, 1H) 7.82 (br. s., 0.5H) 7.56 (br. s., 0.5H) 7.43 (br. s., 1H) 5.35-5.70 (m, 1H) 3.76 (d, 3H) 1.67 (d, 3H).

LC-MS: 367 [M+H]⁺.

### Example 10(b)

### Second Eluting Compound

### N²-[1-(5-Fluoropyrimidin-2-yl)ethyl]-N⁴-(1-methyl-1H-imidazol-4-yl)pteridine-2,4-diamine, Enantiomer (B)

The second eluting compound had a retention time of 15.52 minutes, >98% ee.

¹H NMR (300 MHz, MeOD) δ ppm 8.49-8.88 (m, 3H) 8.37 (d, 1H) 7.88 (br. s., 0.5H) 7.56 (br. s., 0.5H) 7.46 (d, 1H) 5.37-5.70 (m, 1H) 3.81 (d, 3H) 1.67 (d, 3H).

LC-MS: 367 [M+H]⁺.

### Example 11

### N⁶-[(1S)-1-(5-Fluoropyrimidin-2-yl)ethyl]-1-methyl-N⁴-(1-methyl-1H-imidazol-4-yl)-1H-pyrazolo[3,4-d]pyrimidine-4,6-diamine

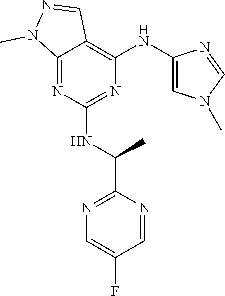

6-Chloro-1-methyl-N-(1-methyl-1H-imidazol-4-yl)-1H-pyrazolo[3,4-d]pyrimidin-4-amine (Intermediate 22, 2 g, 7.58 mmol) and (1S)-1-(5-fluoropyrimidin-2-yl)ethanamine hydrochloride (Intermediate 6, 1.482 g, 8.34 mmol) were suspended in butan-1-ol (21.05 ml) and TEA (4.23 ml, 30.34 mmol) was added. The reaction mixture was subjected to microwave irradiation at 180° C. for 3 hours. The reaction mixture was filtered and the filtrate was concentrated in vacuo leaving a brown semi-solid (3.504 g). This material was purified by ISCO (5% MeOH/DCM, isocratic). Concentration of the fractions in vacuo provided the title product as part of a mixture of enantiomers (1.579 g) in the form of a yellow solid, the title enantiomer being present in the mixture in an amount greater than or equal to the amount of the corresponding R enantiomer.

¹H NMR (300 MHz, MeOD) δ ppm 8.69 (s, 2H) 7.85 (br. s., 1H) 7.53 (br. s., 1H) 7.42 (s, 1H) 5.42 (q, 1H) 3.65-3.89 (m, 6H) 1.61 (d, 3H).

LCMS: 369[M+H]⁺.

### Column and Solvent Conditions

The R and S enantiomers were separated using chiral HPLC (Chiralpak® AD column).

Column dimensions: 50×500 cm, 20μ
Mobile phase: 100% MeOH
Flow rate (ml/min): 120 mL/min

### Detection (nm): 220 nm

### Post Purification Purity Check

Sample purity was checked by SFC with a AD column.

Column dimensions: 4.6×100 mm, 5μ
Modifier: 40% Methanol with 0.1% Dimethylethylamine
Flow: 5 mL/min

### Outlet Pressure: 120 bar

### Detection: 220 nm

### Example 11(a)

### First Eluting Compound

### N⁶-[1-(5-Fluoropyrimidin-2-yl)ethyl]-1-methyl-N⁴-(1-methyl-1H-imidazol-4-yl)-1H-pyrazolo[3,4-d]pyrimidine-4,6-diamine, Enantiomer (A)

The first eluting compound had a retention time of 1.34 minutes, 93.1% ee.

¹H NMR (300 MHz, MeOD) δ ppm 8.69 (s, 2H) 7.84 (br. s., 1H) 7.53 (br. s., 1H) 7.43 (s, 1H) 5.42 (q, 1H) 3.60-3.89 (m, 6H) 1.61 (d, 3H).

LCMS: 369 [M+H]⁺.

### Example 11(b)

### Second Eluting Compound

### N⁶-[1-(5-Fluoropyrimidin-2-yl)ethyl]1-methyl-N⁴-(1-methyl-1H-imidazol-4-yl)-1H-pyrazolo[3,4-d]pyrimidine-4,6-diamine, Enantiomer (B)

The second eluting compound had a retention time of 2.30 minutes, >98% ee.

¹H NMR (300 MHz, MeOD) δ ppm 8.69 (s, 2H) 7.84 (br. s., 1H) 7.53 (br. s., 1H) 7.42 (s, 1H) 5.42 (q, 1H) 3.59-3.90 (m, 6H) 1.60 (d, 3H).

LCMS: 369 [M+H]⁺.

### Example 12

### N²-[1-(3,5-Difluoropyridin-2-yl)ethyl]-N⁴-(1-methyl-1H-imidazol-4-yl)pyrido[2,3-d]pyrimidine-2,4-diamine, Trifluoroacetic Acid Salt

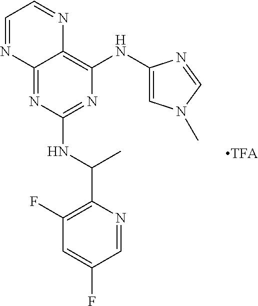

2-Chloro-N-(1-methyl-1H-imidazol-4-yl)pyrido[2,3-d]pyrimidin-4-amine (Intermediate 23, 260 mg, 1.00 mmol) and 1-(3,5-difluoropyridin-2-yl)ethanamine hydrochloride (Intermediate 35, 158 mg, 1.00 mmol) were suspended in n-BuOH (5 mL) followed by the addition of TEA (0.209 mL, 1.5 mmol). The reaction mixture was irradiated in a microwave at 170° C. for 5 hours. Evaporation of the volatiles under vacuum gave a residue, which was purified by reversed phase HPLC (Gilson® chromatography, MeCN/0.1% TFA in water 5%→45%) to afford the title product as a racemic mixture (130 mg).

¹H NMR (300 MHz, MeOD) δ ppm 8.79 (d., 1H) 8.24 (d, 1H), 8.40 (d, 1H) 7.81 (s, 1H) 7.68 (dd., 1H) 7.59 (d, 1H) 7.52 (dd, 1H) 5.74 (q, 1H) 3.90 (s, 3H) 1.698 (d, 3H).

LCMS: 383 [M+H]⁺.

### Column and Solvent Conditions

The R and S enantiomers were separated using chiral HPLC (Chiralpak® AD column).

Column dimensions: 5×50 cm, 20μ
Mobile phase: methanol/ethanol:diethylamine (80:20:0.1 Hexane:(1:1))
Flow rate (ml/min): 120 mL/min

### Detection (nm): 240 nm

### Post Purification Purity Check

Sample purity was checked with Chiralpak® AD-H column.

Column dimensions: 2.5×250 mm, 10μ
Mobile phase: 80:20:0.1 Hexane:(1:1)methanol/ethanol:diethylamine
Flow: 1.0 mL/min

### Detection: 240 nm

### Example 12(a)

### First Eluting Compound

### N²-[1-(3,5-Difluoropyridin-2-yl)ethyl]-N⁴-(1-methyl-1H-imidazol-4-yl)pyrido[2,3-d]pyrimidine-2,4-diamine, Enantiomer A

The first eluting compound had a retention time of 12.21 minutes, >98% ee.

¹H NMR (300 MHz, MeOD) δ ppm 8.53 (bs., 1H) 8.24 (d, 1H) 8.40 (d, 1H) 7.57 (bs, 1H) 7.47 (dd., 1H) 7.24 (d, 1H) 7.07 (dd, 1H) 5.74 (q, 1H) 3.58 (bs, 3H) 1.50 (d, 3H).

LCMS: 383 [M+H]⁺

### Example 12(b)

### Second Eluting Compound

### N²-[1-(3,5-Difluoropyridin-2-yl)ethyl]-N⁴-(1-methyl-1H-imidazol-4-yl)pyrido[2,3-d]pyrimidine-2,4-diamine, Enantiomer B

The second eluting compound had a retention time of 19.09 minutes, >98% ee.

¹H NMR (300 MHz, MeOD) δ ppm 8.53 (s., 1H) 8.24 (d, 1H) 8.41 (d, 1H) 8.24 (d, 1H) 7.63 (bs, 1H) 7.46 (dd., 1H) 7.33 (bs, 1H) 7.05 (dd, 1H) 5.69 (q, 1H) 3.65 (bs, 3H) 1.50 (d, 3H).

LCMS: 383 [M+H]⁺.

### Example 13

### N⁶-[1-(3,5-Difluoropyridin-2-yl)-2-methoxyethyl]-1-methyl-N⁴-(1-methyl-1H-imidazol-4-yl)-1H-pyrazolo[3,4-d]pyrimidine-4,6-diamine, Trifluoroacetic Acid Salt

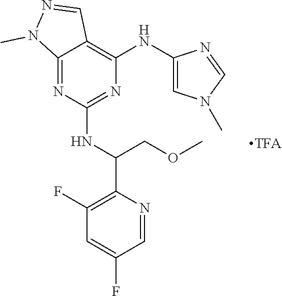

6-Chloro-1-methyl-N-(1-methyl-1H-imidazol-4-yl)-1H-pyrazolo[3,4-d]pyrimidin-4-amine (Intermediate 22, 270 mg, 1.02 mmol) and (1R)-1-(3,5-difluoropyridin-2-yl)-2-methoxyethanamine, (R)-mandelic acid salt (Intermediate 32, 193 mg, 1.02 mmol) were reacted using a procedure similar to the one described for the synthesis of Example 12. After purification by reversed phase HPLC (Gilson® chromatography, (MeCN/0.1% TFA in water 5%→55%), the title product was provided as a racemic mixture, in the form of a yellow solid (340 mg).

¹H NMR (300 MHz, MeOD) δ ppm 8.52 (s., 1H) 8.37 (d, 1H) 7.91 (s, 1H) 7.60 (ddd., 1H) 7.37 (s, 1H) 5.79 (t, 1H) 3.79-3.92 (m, 8H) 3.40 (s, 3H).

LCMS: 416 [M+H]⁺.

### Column and Solvent Conditions

The R and S enantiomers were separated using chiral HPLC (Chiralpak® AD column).

Column dimensions: 5×50 cm, 20μ
Mobile phase: 80:20:0.1 Hexane:(1:1)methanol/ethanol:diethylamine
Flow rate (ml/min): 120 mL/min

### Detection (nm): 240 nm

### Post Purification Purity Check

Sample purity was checked with Chiralpak® AD-H column.

Column dimensions: 2.5×250 mm, 10μ
Mobile phase: 80:20:0.1 Hexane:(1:1) methanol/ethanol:diethylamine
Flow: 1.0 mL/min

### Detection: 240 nm

### Example 13(a)

### First Eluting Compound

### N⁶-[1-(3,5-Difluoropyridin-2-yl)-2-methoxyethyl]-1-methyl-1H-imidazol-4-yl)-1H-pyrazolo[3,4-d]pyrimidine-4,6-diamine, Enantiomer (A)

The first eluting compound had a retention time of 1.27 minutes, >98% ee.

LCMS: 416 [M+H]⁺.

### Example 13(b)

### Second Eluting Compound N⁶-[1-(3,5-Difluoropyridin-2-yl)-2-methoxyethyl]-1-methyl-N⁴-(1-methyl-1H-imidazol-4-yl)-1H-pyrazolo[3,4-d]pyrimidine-4,6-diamine, Enantiomer (B)

The second eluting compound had a retention time of 2.06 minutes, >98% ee.

¹H NMR (300 MHz, MeOD) δ ppm 8.52 (brs, 1H) 8.38 (d, 1H) 7.90 (s, 1H) 7.60 (ddd, 1H) 7.37 (br.s, 1H) 5.84 (t, 1H) 3.76-3.92 (m, 8H) 3.37 (s, 3H).

LCMS: 416 [M+H]⁺.

### Example 14

### N⁶-[1-(3,5-Difluoropyridin-2-yl)ethyl]-1-methyl-N⁴-(1-methyl-1H-imidazol-4-yl)-1H-pyrazolo[3,4-d]pyrimidine-4,6-diamine, Trifluoroacetic Acid Salt

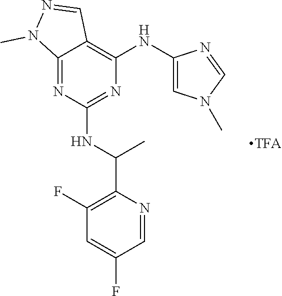

6-Chloro-1-methyl-N-(1-methyl-1H-imidazol-4-yl)-1H-pyrazolo[3,4-d]pyrimidin-4-amine (Intermediate 22, 300 mg, 1.14 mmol) and 1-(3,5-difluoropyridin-2-yl)ethanamine hydrochloride (Intermediate 35, 180 mg, 1.14 mmol) were reacted using a procedure similar to the one described for the synthesis of Example 12. After purification by reversed phase HPLC (Gilson® chromatography, MeCN/0.1% TFA in water 5-45%), the title product was provided as a racemic mixture in the form of a solid (150 mg).

¹H NMR (300 MHz, MeOD) δ ppm 8.47 (s., 1H) 8.34 (d, 1H) 7.89 (s, 1H) 7.59 (ddd., 1H) 7.35 (s, 1H) 5.60 (q, 1H) 3.95 (s, 3H) 3.80 (s, 3H) 1.60 (d, 3H).

LCMS: 386 [M+H]⁺.

### Column and Solvent Conditions

The R and S enantiomers were separated using chiral HPLC (Chiralpak® AD column).

Column dimensions: 5×50 cm, 20μ
Mobile phase: 80:20:0.1 Hexane:(1:1)methanol/ethanol:diethylamine
Flow rate (ml/min): 120 mL/min

### Detection (nm): 240 nm

### Post Purification Purity Check

Sample purity was checked with Chiralpak® AD-H column.

Column dimensions: 2.5×250 mm, 10μ
Mobile phase: 80:20:0.1 Hexane:(1:1)methanol/ethanol:diethylamine
Flow: 1.0 mL/min

### Detection: 240 nm

### Example 14(a)

### First Eluting Compound

### N⁶-[1-(3,5-Difluoropyridin-2-yl)ethyl]-1-methyl-N⁴-(1-methyl-1H-imidazol-4-yl)-1H-pyrazolo[3,4-d]pyrimidine-4,6-diamine, Enantiomer (A)

The first eluting compound had a retention time of 1.60 minutes, >98% ee.

¹H NMR (300 MHz, MeOD) δ ppm 8.47 (s., 1H) 8.34 (d., 1H) 7.52-7.63 (m, 2H) 7.46 (s, 1H) 5.63 (q, 1H) 3.79 (s, 6H) 1.57 (d, 3H).

LCMS: 386 [M+H]⁺.

### Example 14(b)

### Second Eluting Compound

### N⁶-[1-(3,5-Difluoropyridin-2-yl)ethyl]-1-methyl-N⁴-(1-methyl-1H-imidazol-4-yl)-1H-pyrazolo[3,4-d]pyrimidine-4,6-diamine, Enantiomer (B)

The second eluting compound had a retention time of 2.29 minutes, >98% ee.

¹H NMR (300 MHz, MeOD) δ ppm 8.47 (s., 1H) 8.21 (s., 1H) 7.41-7.51 (m, 2H) 7.34 (s, 1H) 5.53 (q, 1H) 3.67 (s, 6H) 1.47 (d, 3H).

LCMS: 386 [M+H]⁺.

### Example 15

### 2-(6-{[(1S)-1-(5-Fluoropyrimidin-2-yl)ethyl]amino}-4-[(1-methyl-1H-imidazol-4-yl)amino]-1H-pyrazolo[3,4-d]pyrimidin-1-yl)ethanol, Trifluoroacetic Acid Salt

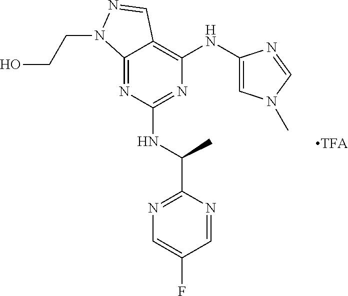

To a solution of 2-{4-[(1-methyl-1H-imidazol-4-yl)amino]-6-(methylsulfonyl)-1H-pyrazolo[3,4-d]pyrimidin-1-yl}ethanol (Intermediate 25, 337 mg, 1 mmol) and (1S)-1-(5-fluoropyrimidin-2-yl)ethanamine hydrochloride (Intermediate 6, 178 mg, 1.00 mmol) in NMP (5 mL) was added TEA (0.139 mL, 1.00 mmol). The reaction mixture was heated at 160° C. overnight. Evaporation of the volatiles under reduced pressure followed by purification by reversed phase HPLC (Gilson® chromatography, MeCN/H₂O (0.1% TFA) 0→55%) provided the title product as part of a mixture of enantiomers (25.1 mg), the title enantiomer being present in the mixture in an amount greater than or equal to the amount of the corresponding R enantiomer.

¹H NMR (300 MHz, MeOD) δ ppm 8.68-8.75 (m, 2H) 8.46 (s, 1H) 7.92 (s, 1H) 7.37 (s., 1H) 5.39 (q, 1H) 3.26 (t, 2H) 3.96 (s, 3H) 3.86 (t, 2H) 1.65 (d, 3H).

LCMS: 399 [M+H]⁺.

### Example 16

### N²-[(1S)-1-(5-Fluoropyrimidin-2-yl)ethyl]-N⁴-(1-methyl-1H-imidazol-4-yl)pyrido[2,3-d]pyrimidine-2,4-diamine, Trifluoroacetic Acid Salt

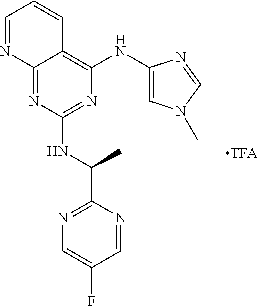

2-Chloro-N-(1-methyl-1H-imidazol-4-yl)pyrido[2,3-d]pyrimidin-4-amine (Intermediate 23, 260 mg, 1.00 mmol) and (1S)-1-(5-fluoropyrimidin-2-yl)ethanamine hydrochloride (Intermediate 6, 177 mg, 1.00 mmol) were reacted using a procedure similar to the one described for the synthesis of Example 12. After purification by reversed phase HPLC (Gilson® chromatography, 0.1% TFA in water/MeCN 5-45%), the title product was provided as part of a solid mixture of enantiomers (100 mg), the title enantiomer being present in the mixture in an amount greater than or equal to the amount of the corresponding R enantiomer.

¹H NMR (300 MHz, MeOD) δ ppm 8.72 (s., 2H) 8.60-8.68 (m, 1H) 8.51 (dd, 1H) 7.83 (bs., 1H) 7.46 (s, 1H) 7.16 (dd, 1H) 5.54 (q, 1H) 3.79 (bs, 3H) 1.66 (d, 3H).

LCMS: 366 [M+H]⁺.

### Example 17

### N²-[(1S)-1-(5-Fluoropyrimidin-2-yl)ethyl]-N⁴-(1-methyl-1H-imidazol-4-yl)-5,6,7,8-tetrahydropyrido[4,3-d]pyrimidine-2,4-diamine

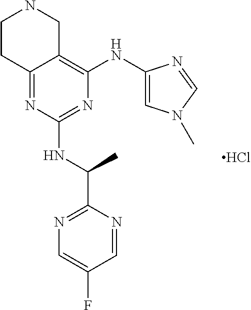

To a solution of tert-butyl 2-chloro-4-[(1-methyl-1H-imidazol-4-yl)amino]-7,8-dihydropyrido[4,3-d]pyrimidine-6(5H)-carboxylate (Intermediate 28, 428 mg, 1.17 mmol) and (1S)-1-(5-fluoropyrimidin-2-yl)ethanamine hydrochloride (Intermediate 6, 208 mg, 1.17 mmol) in n-BuOH (5 mL) was added TEA (0.409 mL, 2.93 mmol). The resulting reaction mixture was heated at 135° C. overnight. The mixture was filtered and the filtrate was concentrated under reduced pressure. The residue left was purified by reversed phase HPLC (Gilson® chromatography) and evaporation of the desired fractions gave the Boc protected intermediate. This material was diluted with methanol and 4N HCl in dioxane was added. The reaction was allowed to stir overnight whereupon evaporation of the volatiles under reduced pressure gave the title product as part of a mixture of enantiomers (260 mg), the title enantiomer being present in the mixture in an amount greater than or equal to the amount of the corresponding R enantiomer.

¹H NMR (300 MHz, MeOD) δ ppm 8.71-8.86 (m, 3H) 7.65 (s, 1H) 5.31 (q, 1H) 4.25 (t, 2H) 4.02 (s, 3H) 3.56-3.70 (m, 2H) 3.14 (t, 2H) 1.66 (d, 3H).

LCMS: 370 [M+H]⁺.

### Column and Solvent Conditions

The R and S enantiomers were separated using chiral HPLC (Chiralpak® AD column).

Column dimensions: 5×50 cm, 20μ
Mobile phase: 50:50:0.1 methanol:ethanol:diethylamine
Flow rate (ml/min): 120 mL/min

### Detection (nm): 254 nm

### Post Purification Purity Check

Sample purity was checked with Chiralpak® AD-H column.

Column dimensions: 4.6×250 mm, 10μ
Mobile phase: 50:50:0.1 methanol:ethanol:diethylamine
Flow: 1.0 mL/min

### Detection: 254 nm

### Example 17 (a)

### First Eluting Compound

### N²-[1-(5-Fluoropyrimidin-2-yl)ethyl]-N⁴-(1-methyl-1H-imidazol-4-yl)-5,6,7,8-tetrahydropyrido[4,3-d]pyrimidine-2,4-diamine, Enantiomer (A)

The first eluting compound had a retention time of 5.83 minutes, >98% ee.

¹H NMR (300 MHz, MeOD) δ ppm 8.71 (s, 2H) 7.41 (bs, 2H) 5.31 (q, 1H) 3.99 (m, 2H) 3.80 (s, 3H) 3.33-3.45 (m, 2H) 2.79 (t, 2H) 1.59 (d, 3H).

LCMS: 370 [M+H]⁺

### Example 17 (b)

### Second Eluting Compound

### N²-[1-(5-Fluoropyrimidin-2-yl)ethyl]-N⁴-(1-methyl-1H-imidazol-4-yl)-5,6,7,8-tetrahydropyrido[4,3-d]pyrimidine-2,4-diamine, Enantiomer (B)

The second eluting compound had a retention time of 17.48 minutes, >98% ee.

¹H NMR (300 MHz, MeOD) δ ppm 8.70 (s, 2H) 7.38 (m, 2H) 5.31 (q, 1H) 3.79 (s, 3H) 3.72 (m, 2H) 3.07-3.13 (m, 2H) 2.62 (t, 2H) 1.58 (d, 3H).

LCMS: 370 [M+H]⁺.

### Example 18

### N²-[(1S)-1-(5-Fluoropyrimidin-2-yl)ethyl]-N⁴-(1-methyl-1H-imidazol-4-yl)-6,7-dihydro-5H-pyrrole[3,4-d]pyrimidine-2,4-diamine, HCl Salt

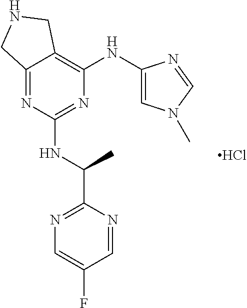

To a solution of tert-butyl 2-chloro-4-[(1-methyl-1H-imidazol-4-yl)amino]-5,7-dihydro-6H-pyrrolo[3,4-d]pyrimidine-6-carboxylate (Intermediate 29, 467 mg, 1.33 mmol) and (1S)-1-(5-fluoropyrimidin-2-yl)ethanamine hydrochloride (Intermediate 6, 236 mg, 1.33 mmol) in n-BuOH (5 mL) was added TEA (0.464 mL, 3.33 mmol). The resulting reaction mixture was heated at 135° C. overnight. The mixture was filtered and the filtrate was concentrated under reduced pressure. The residue left was purified by reversed phase HPLC (Gilson® chromatography) and evaporation of the desired fractions gave the Boc protected intermediate. This material was diluted with methanol and 4N HCl in dioxane was added. The reaction was allowed to stir overnight whereupon evaporation of the volatiles under reduced pressure gave the title product as part of a mixture of enantiomers (168 mg), the title enantiomer being present in the mixture in an amount greater than or equal to the amount of the corresponding R enantiomer.

¹H NMR (300 MHz, MeOD) δ ppm 8.85 (br. s, 1H) 8.75 (s, 2H) 7.62 (s, 1H) 5.34 (q, 1H) 4.56 (s, 4H) 4.02 (s, 3H) 1.66 (d, 3H).

LCMS: 356 [M+H]⁺.

### Column and Solvent Conditions

The R and S enantiomers were separated using chiral HPLC (Chiralpak® AD column).

Column dimensions: 5×50 cm, 20μ
Mobile phase: 50:50:0.1 methanol:ethanol:diethylamine
Flow rate (ml/min): 120 mL/min

### Detection (nm): 254 nm

### Post Purification Purity Check

Sample purity was checked with Chiralpak® AD-H column.

Column dimensions: 4.6×250 mm, 10μ
Mobile phase: 50:50:0.1 methanol:ethanol:diethylamine
Flow: 1.0 mL/min

### Detection: 254 nm

### Example 18(a)

### First Eluting Compound

### N²-[1-(5-Fluoropyrimidin-2-yl)ethyl]-N⁴-(1-methyl-1H-imidazol-4-yl)-6,7-dihydro-5H-pyrrolo[3,4-d]pyrimidine-2,4-diamine, Enantiomer (A)

The first eluting compound had a retention time of 5.83 minutes, >98% ee.

¹H NMR (300 MHz, MeOD) δ ppm 8.85 (br. s, 1H) 8.71 (s, 2H) 7.43 (s, 1H) 5.34 (q, 1H) 4.32 (s, 2H) 4.21 (s, 2H) 3.79 (s, 3H) 1.60 (d, 3H).

LCMS: 356 [M+H]⁺.

### Example 18(b)

### Second Eluting Compound

### N²-[1-(5-Fluoropyrimidin-2-yl)ethyl]-N⁴-(1-methyl-1H-imidazol-4-yl)-6,7-dihydro-5H-pyrrolo[3,4-d]pyrimidine-2,4-diamine, Enantiomer (B)

The second eluting compound had a retention time of 17.48 minutes, >98% ee.

¹H NMR (300 MHz, MeOD) δ ppm 8.85 (br. s, 1H) 8.71 (s, 2H) 7.42 (s, 1H) 5.34 (q, 1H) 4.26 (s, 4H) 4.16 (s, 2H) 3.79 (s, 3H) 1.60 (d, 3H).

LCMS: 356 [M+H]⁺.

### Example 19

### N²-[(1S)-1-(5-Fluoropyrimidin-2-yl)ethyl]-N⁴-(1-methyl-1H-imidazol-4-yl)-6-(trifluoromethyl)-7H-pyrrolo[2,3-d]pyrimidine-2,4-diamine, Trifluoroacetic Acid Salt

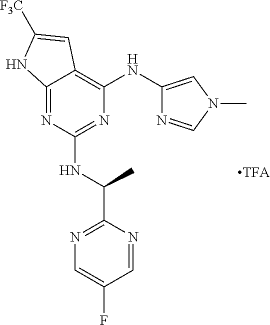

3-Bromo-1,1,1-trifluoropropan-2-one oxime (75 mg, 0.36 mmol) and N²-[(1S)-1-(5-fluoropyrimidin-2-yl)ethyl]-N⁴-(1-methyl-1H-imidazol-4-yl)pyrimidine-2,4,6-triamine (Intermediate 37, 100 mg, 0.30 mmol) in DMF (1518 μl) was heated to 110° C. The reaction mixture was diluted with DCM/MeOH and washed with water. After concentration under reduced pressure, the residue was purified by reversed phase HPLC (Gilson® chromatography, 5%→55% MeCN/water 0.1% TFA). Concentration of fractions under reduced pressure provided the title product as a part of a mixture of enantiomers (6.49 mg), the title enantiomer being present in the mixture in an amount greater than or equal to the amount of the corresponding R enantiomer.

¹H NMR (300 MHz, MeOD) δ ppm 8.72 (s, 2H), 8.25 (s, 1H), 7.26 (s, 1H), 6.96 (d, 1H), 5.36 (q, 1H), 3.92 (s, 3H), 1.64 (d, 3H).

LCMS: 422 [M+H]⁺.

### Example 20

### N²-[(1S)-1-(5-Fluoropyrimidin-2-yl)ethyl]-6-methyl-N⁴-(1-methyl-1H-imidazol-4-yl)-7H-pyrrolo[2,3-d]pyrimidine-2,4-diamine, Trifluoroacetic Acid Salt

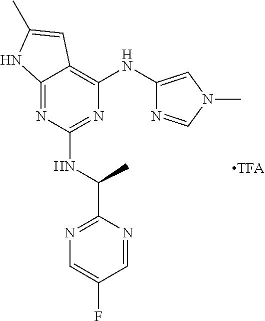

A solution of N²-[(1S)-1-(5-Fluoropyrimidin-2-yl)ethyl]-6-methyl-N⁴-(1-methyl-1H-imidazol-4-yl)-7-[(4-methylphenyl)sulfonyl]-7H-pyrrolo[2,3-d]pyrimidine-2,4-diamine (Example 21, 170 mg, 0.33 mmol) and aq. sodium hydroxide (978 μl, 1.96 mmol) in 1,4-dioxane (652 μl) was heated at 120° C. for 80 min. The reaction mixture was diluted with water and extracted with DCM/MeOH. Concentration of organic layers was followed by purification by reversed phase HPLC (Gilson® chromatography, 5%→50% MeCN/water with 0.1% TFA) to yield the title product as part of a mixture of enantiomers (9 mg), the title enantiomer being present in the mixture in an amount greater than or equal to the amount of the corresponding R enantiomer.

¹H NMR (300 MHz, MeOD) δ ppm 8.67 (s, 2H), 7.43 (d, 1H), 7.37 (s, 1H), 6.00 (s, 1H), 5.37 (q, 1H), 3.78 (s, 3H), 2.27 (s, 3H), 1.58 (d, 3H).

LCMS: 368 [M+H]⁺.

### Example 21

### N²-[(1S)-1-(5-Fluoropyrimidin-2-yl)ethyl]-6-methyl-N⁴-(1-methyl-1H-imidazol-4-yl)-7-[(4-methylphenyl)sulfonyl]-7H-pyrrolo[2,3-d]pyrimidine-2,4-diamine

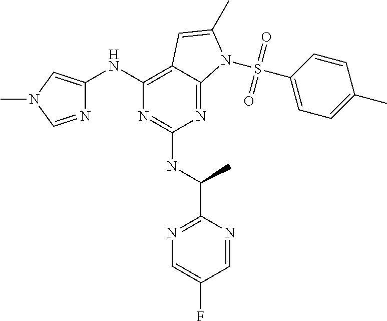

(1S)-1-(5-Fluoropyrimidin-2-yl)ethanamine hydrochloride (Intermediate 6, 1022 mg, 5.76 mmol) and 2-chloro-6-methyl-N-(1-methyl-1H-imidazol-4-yl)-7-[(4-methylphenyl)sulfonyl]-7H-pyrrolo[2,3-d]pyrimidin-4-amine (Intermediate 41, 600 mg, 1.44 mmol) and DIPEA (2514 μl, 14.39 mmol) in n-BuOH (2284 μl) was heated under microwave irradiation for 5 hours at 160° C. The reaction mixture was diluted with DCM/MeOH and washed with water. The organic extracts were concentrated under reduced pressure to give a residue, which was purified by reversed phase HPLC (Gilson® chromatography, 5%→80% H₂O/MeCN 0.1% ammonia acetate) to give the title product as part of a mixture of enantiomers (170 mg), the title enantiomer being present in the mixture in an amount greater than or equal to the amount of the corresponding R enantiomer.

LCMS: 522 [M+H]⁺.

### Example 22

### 7-(2-Fluoroethyl)-N²-[(1S)-1-(5-fluoropyrimidin-2-yl)ethyl]-N⁴-(1-methyl-1H-imidazol-4-yl)-7H-pyrrolo[2,3-d]pyrimidine-2,4-diamine, Trifluoroacetic Acid Salt

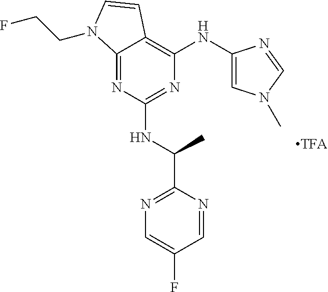

A mixture of (1S)-1-(5-fluoropyrimidin-2-yl)ethanamine hydrochloride (Intermediate 6, 546 mg, 3.07 mmol), 2-chloro-7-(2-fluoroethyl)-N-(1-methyl-1H-imidazol-4-yl)-7H-pyrrolo[2,3-d]pyrimidin-4-amine (Intermediate 43, 453 mg, 1.54 mmol), palladium(II) acetate (34.5 mg, 0.15 mmol), (R)-(−)-1-[(S)-2-(dicyclohexylphosphino) ferrocenyl]ethyldi-t-butylphosphine] (134 mg, 0.25 mmol) and CS₂CO₃ (3506 mg, 10.76 mmol) in 1,4-dioxane (7685 μl) was heated at 150° C. for 25 min with well-stirring in microwave reactor. The reaction mixture was diluted with DCM/MeOH (10%) and the organic layer was washed with water. Concentration of the organic layer under reduced pressure provided residue, which was purified by reversed phase HPLC (Gilson® chromatography, 5%→45% MeCN/0.1 TFA H₂O) to provide the title product as part of a mixture of enantiomers (211 mg), the title enantiomer being present in the mixture in an amount greater than or equal to the amount of the corresponding R enantiomer.

¹H NMR (300 MHz, MeOD) δ ppm 8.69 (s, 2H), 7.50 (d, 1H), 7.40 (s, 1H), 6.76 (d, 1H), 6.38 (d, J=3.58 Hz, 1H), 5.37 (q, 1H), 4.61-4.75 (m, 1H), 4.43-4.59 (m, 1H), 4.28-4.38 (m, 1H), 4.14-4.27 (m, 1H), 3.79 (s, 3H), 1.60 (d, 3H).

LCMS: 400 [M+H]⁺.

### Column and Solvent Conditions

The R and S enantiomers were separated using Chiral SFC (Chiralpak® AD column).

Column dimensions: 21×250 mm, 5μ
Mobile phase: 65%:35%:0.4% CarbonDioxide:Methanol:dimethylethylamine
Flow rate (ml/min): 60

### Detection (nm): 254

### Post Purification Purity Check

Sample purity was checked with Chiralpak® AD.

Column dimensions: 4.6×100 mm, 5μ
Mobile phase: 70%:30%:0.4% CarbonDioxide:Methanol:dimethylethylamine
Flow: 1.0 mL/min

### Detection: 254 nm

### Example 22(a)

### First Eluting Compound 7-(2-Fluoroethyl)-N²-[1-(5-fluoropyrimidin-2-yl)ethyl]-N⁴-(1-methyl-1H-imidazol-4-yl)-7H-pyrrolo[2,3-d]pyrimidine-2,4-diamine, Enantiomer (A)

The first eluting compound had a retention time of 2.05 minutes, >98% ee.

¹H NMR (300 MHz, MeOD) δ ppm 8.69 (s, 2H), 7.50 (d, 1H), 7.40 (s, 1H), 6.76 (d, J=3.58 Hz, 1H), 6.38 (d, 1H), 5.37 (q, 1H), 4.61-4.75 (m, 1H), 4.43-4.59 (m, 1H), 4.28-4.38 (m, 1H), 4.14-4.27 (m, 1H), 3.79 (s, 3H), 1.60 (d, 3H).

LCMS: 400 [M+H]⁺.

### Example 22(b)

### Second Eluting Compound

### 7-(2-Fluoroethyl)-N²-[1-(5-fluoropyrimidin-2-yl)ethyl]-N⁴-(1-methyl-1H-imidazol-4-yl)-7H-pyrrolo[2,3-d]pyrimidine-2,4-diamine, Enantiomer (B)

The second eluting compound had a retention time of 2.62 minutes, >98% ee.

¹H NMR (300 MHz, MeOD) δ ppm 8.69 (s, 2H), 7.50 (d, 1H), 7.40 (s, 1H), 6.76 (d, 1H), 6.38 (d, 1H), 5.37 (q, 1H), 4.61-4.75 (m, 1H), 4.43-4.59 (m, 1H), 4.28-4.38 (m, 1H), 4.14-4.27 (m, 1H), 3.79 (s, 3H), 1.60 (d, 3H).

LCMS: 400 [M+H]⁺.

### Example 23

### N²-[(1S)-1-(5-Fluoropyrimidin-2-yl)ethyl]-7-methyl-N⁴-(1-methyl-1H-imidazol-4-yl)-7H-pyrrole[2,3-d]pyrimidine-2,4-diamine, Trifluoroacetic Acid Salt

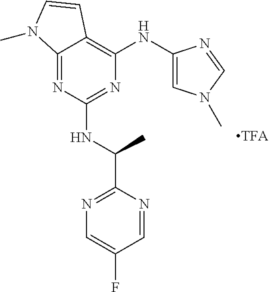

(1S)-1-(5-Fluoropyrimidin-2-yl)ethanamine hydrochloride (Intermediate 6, 303 mg, 1.71 mmol) and 2-chloro-7-methyl-N-(1-methyl-1H-imidazol-4-yl)-7H-pyrrolo[2,3-d]pyrimidin-4-amine, trifluoroacetic acid salt (Intermediate 45, 300 mg, 1.14 mmol) were reacted using a procedure similar to the one described for the synthesis of Example 22. After purification by reversed phase HPLC (Gilson® chromatography, MeCN/0.1% TFA in water 5%→45%) the title product was provided as part of a mixture of enantiomers (211 mg), the title enantiomer being present in the mixture in an amount greater than or equal to the amount of the corresponding R enantiomer.

¹H NMR (300 MHz, MeOD) δ ppm 8.58 (s, 2H), 7.36 (d, 1H), 7.28 (d, 1H), 6.58 (d, 1H), 6.26 (d, 1H), 5.30 (q, 1H), 3.68 (s, 3H), 3.47 (s, 3H), 1.50 (d, 3H).

LCMS: 368 [M+H]⁺.

### Column and Solvent Conditions

The R and S enantiomers were separated using Chiral SFC (Chiralpak® AD column):

Column dimensions: 21×250 mm, 5μ
Mobile phase: 65%:35%:0.4% CarbonDioxide:Methanol:dimethylethylamine
Flow rate (ml/min): 60

### Detection (nm): 254

### Post Purification Purity Check

Sample purity was checked with Chiralpak® AD.

Column dimensions: 4.6×250 mm, 5μ
Mobile phase: 60%:40%:0.4% CarbonDioxide:Methanol:dimethylethylamine
Flow: 2.5 mL/min

### Detection: 254 nm

### Example 23(a)

### First Eluting Compound

### N²-[1-(5-Fluoropyrimidin-2-yl)ethyl]-7-methyl-N⁴-(1-methyl-1H-imidazol-4-yl)-7H-pyrrolo[2,3-d]pyrimidine-2,4-diamine, Enantiomer (A)

The first eluting compound had a retention time of 4.33 minutes, >98% ee.

¹H NMR (300 MHz, MeOD) δ ppm 8.58 (s, 2H), 7.36 (d, 1H), 7.28 (d, 1H), 6.58 (d, 1H), 6.26 (d, 1H), 5.30 (q, 1H), 3.68 (s, 3H), 3.47 (s, 3H), 1.50 (d, 3H).

LCMS: 368 [M+H]⁺.

### Example 23(b)

### Second Eluting Compound

### N²-[1-(5-Fluoropyrimidin-2-yl)ethyl]-7-methyl-N⁴-(1-methyl-1H-imidazol-4-yl)-7H-pyrrolo[2,3-d]pyrimidine-2,4-diamine, Enantiomer (B)

The second eluting compound had a retention time of 5.77 minutes, >98% ee

¹H NMR (300 MHz, MeOD) δ ppm 8.58 (s, 2H), 7.36 (d, 1H), 7.28 (d, 1H), 6.58 (d, 1H), 6.26 (d, 1H), 5.30 (q, 1H), 3.68 (s, 3H), 3.47 (s, 3H), 1.50 (d, 3H).

LCMS: 368 [M+H]⁺.

### Example 24

### 7-Cyclopropyl-N²-[(1S)-1-(5-fluoropyrimidin-2-yl)ethyl]-N⁴-(1-methyl-1H-imidazol-4-yl)-7H-pyrrolo[2,3-d]pyrimidine-2,4-diamine, Trifluoroacetic Acid Salt

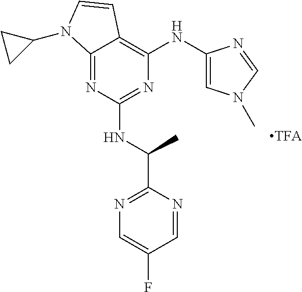

(1S)-1-(5-Fluoropyrimidin-2-yl)ethanamine hydrochloride (Intermediate 6, 252 mg, 1.42 mmol) and 2-chloro-7-cyclopropyl-N-(1-methyl-1H-imidazol-4-yl)-7H-pyrrolo[2,3-d]pyrimidin-4-amine, trifluoroacetic acid salt (Intermediate 47, 205 mg, 0.71 mmol) were reacted using a procedure similar to the one described for the synthesis of Example 22. After purification by reversed phase HPLC (Gilson® chromatography, MeCN/0.1% TFA in water 5%→45%), the title product was provided as part of a mixture of enantiomers (40 mg), the title enantiomer being present in the mixture in an amount greater than or equal to the amount of the corresponding R enantiomer.

¹H NMR (300 MHz, MeOD) δ ppm 8.70 (s, 2H), 7.47 (d, 1H), 7.39 (d, 1H), 6.70 (d, 1H), 6.33 (d, 1H), 5.27-5.52 (m, 1H), 3.80 (s, 3H), 3.17-3.29 (m, 1H), 1.62 (d, 3H), 0.74-1.07 (m, 4H).

LCMS 394 [M+H]⁺.

### Column and Solvent Conditions

The R and S enantiomers were separated using Chiral SFC (Chiralpak® AD column).

Column dimensions: 21×250 mm, 5μ
Mobile phase: 75%:25%:0.4% CarbonDioxide:Methanol:dimethylethylamine
Flow rate (ml/min): 60

### Detection (nm): 254

### Post Purification Purity Check

Sample purity was checked with Chiralpak® AD.

Column dimensions: 4.6×100 mm, 5μ
Mobile phase: 80%:20%:0.4% CarbonDioxide:Methanol:dimethylethylamine
Flow: 5.0 mL/min

### Detection: 254 nm

### Example 24(a)

### First Eluting Compound

### 7-Cyclopropyl-N²-[1-(5-fluoropyrimidin-2-yl)ethyl]-N⁴-(1-methyl-1H-imidazol-4-yl)-7H-pyrrolo[2,3-d]pyrimidine-2,4-diaminev, Enantiomer (A)

The first eluting compound had a retention time of 3.44 minutes, >98% ee.

LCMS: 394 [M+H]⁺

Not enough material was isolated for full characterization.

### Example 24(b)

### Second Eluting Compound

### 7-Cyclopropyl-N²-[1-(5-fluoropyrimidin-2-yl)ethyl]-N⁴-(1-methyl-1H-imidazol-4-yl)-7H-pyrrolo[2,3-d]pyrimidine-2,4-diamine, Enantiomer (B)

The second eluting compound had a retention time of 4.10 minutes, >98% ee

¹H NMR (300 MHz, MeOD) δ ppm 8.70 (s, 2H), 7.47 (d, 1H), 7.39 (d, 1H), 6.70 (d, 1H), 6.33 (d, 1H), 5.27-5.52 (m, 1H), 3.80 (s, 3H), 3.17-3.29 (m, 1H), 1.62 (d, 3H), 0.74-1.07 (m, 4H).

LCMS: 394 [M+H]⁺.

### Example 25

### N²-[1-(3,5-Difluoropyridin-2-yl)ethyl]-N⁴-(1-methyl-1H-imidazole-4-yl)-7H-pyrrolo[2,3-d]pyrimidine-2,4-diamine, Trifluoroacetic Acid Salt

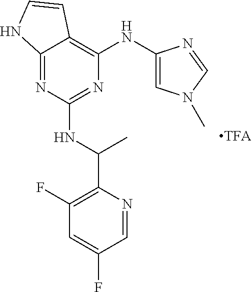

A solution of N²-[1-(3,5-difluoropyridin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine (Example 26, 600 mg, 1.14 mmol) and KOH (1925 mg, 34.32 mmol) in water (4.00 mL), methanol (0.400 mL) and THF (2 mL) was heated at 55° C. overnight. The reaction mixture was acidified with HCl and subsequently neutralized with aq. saturated NaHCO₃. Extraction of aqueous layer with DCM/MeOH (10%) was followed by concentration of the organic layer under reduced pressure to provide a residue, which was purified by reversed phase HPLC (Gilson® chromatography, 5%→65% MeCN/0.1% TFA in water) to give the title product as a racemic mixture.

¹H NMR (300 MHz, MeOD) δ ppm 8.31 (d, 1H), 7.48-7.57 (m, 1H), 7.44 (s, 1H), 7.40 (s, 1H), 6.73 (d, 1H), 6.36 (d, 1H), 5.53-5.67 (m, 1H), 3.77 (s, 3H), 1.53 (d, 3H).

LCMS: 370 [M+H]⁺.

### Column and Solvent Conditions

The R and S enantiomers were separated using Chiral SFC (Chiralpak® AD column).

Column dimensions: 21×250 mm, 5μ
Mobile phase: 55%:45%:0.4% CarbonDioxide:Methanol:dimethylethylamine
Flow rate (ml/min): 60

### Detection (nm): 220

### Post Purification Purity Check

Sample purity was checked with Chiralpak® AD.

Column dimensions: 4.6×250 mm, 5μ
Mobile phase: 55%:45%:0.4% CarbonDioxide:Methanol:dimethylethylamine Flow: 2.5 mL/min

### Detection: 220 nm

### Example 25(a)

### First Eluting Compound

### N²-[1-(3,5-Difluoropyridin-2-yl)ethyl]-N⁴-(1-methyl-1H-imidazol-4-yl)-7H-pyrrolo[2,3-d]pyrimidine-2,4-diamine, Enantiomer (A)

The first eluting compound had a retention time of 3.83 minutes, >98% ee.

¹H NMR (300 MHz, MeOD) δ ppm 8.31 (d, J=2.26 Hz, 1H), 7.48-7.57 (m, 1H), 7.44 (s, 1H), 7.40 (s, 1H), 6.73 (d, J=3.58 Hz, 1H), 6.36 (d, J=3.39 Hz, 1H), 5.53-5.67 (m, 1H), 3.77 (s, 3H), 1.53 (d, J=6.78 Hz, 3H).

### Example 25(b)

### Second Eluting Compound

### N²-[1-(3,5-Difluoropyridin-2-yl)ethyl]-N⁴-(1-methyl-1H-imidazol-4-yl)-7H-pyrrolo[2,3-d]pyrimidine-2,4-diamine, Enantiomer (B)

The second eluting compound had a retention time of 7.75 minutes, >98% ee.

¹H NMR (300 MHz, MeOD) δ ppm 8.31 (d, J=2.26 Hz, 1H), 7.48-7.57 (m, 1H), 7.44 (s, 1H), 7.40 (s, 1H), 6.73 (d, J=3.58 Hz, 1H), 6.36 (d, J=3.39 Hz, 1H), 5.53-5.67 (m, 1H), 3.77 (s, 3H), 1.53 (d, J=6.78 Hz, 3H)

### Example 26

### N²-[1-(3,5-Difluoropyridin-2-yl)ethyl]-N⁴-(1-methyl-1H-imidazol-4-yl)-7-[(4-methylphenyl)sulfonyl]-7H-pyrrolo[2,3-d]pyrimidine-2,4-diamine

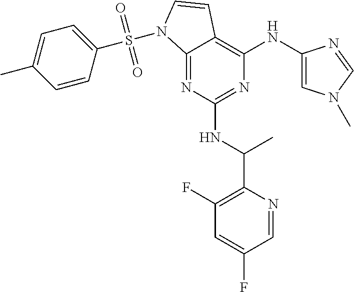

A mixture of 1-(3,5-difluoropyridin-2-yl)ethanamine hydrochloride (Intermediate 35, 998 mg, 4.32 mmol), 2-chloro-N-(1-methyl-1H-imidazol-4-yl)-7-[(4-methylphenyl)sulfonyl]-7H-pyrrolo[2,3-d]pyrimidin-4-amine (Intermediate 10, 870 mg, 2.16 mmol), and DIPEA (1509 μl, 8.64 mmol) in n-BuOH (2810 μl) was heated at 180° C. for 5 hours. The volatiles were concentrated under reduced pressure to give a residue, which was purified utilizing ISCO (0%→100% DCM/EtOAc) to yield the title product (600 mg, 53%) as a racemic mixture.

LCMS: 524 [M+H]⁺.

### Example 27

### N²-[1-(5-Methoxypyrimidin-2-yl)ethyl]-N⁴-(1-methyl-1H-imidazol-4-yl)-7H-pyrrolo[2,3-d]pyrimidine-2,4-diamine

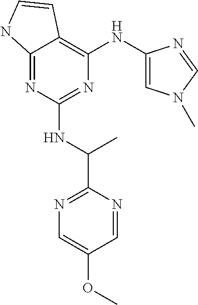

The title product was obtained as a by-product, as a mixture of enantiomers, of the reaction used for the synthesis of Example 4 (53 mg, 1%).

¹H NMR (300 MHz, MeOD) δ ppm 8.49 (s, 2H), 8.04 (br. s., 1H), 7.26 (s, 1H), 6.97 (d, 1H), 6.61 (d, 1H), 5.30 (q, 1H), 3.95 (s, 3H), 3.89 (s, 3H), 1.64 (d, 3H).

LCMS: 365 [M+H]⁺.

### Column and Solvent Conditions

The R and S enantiomers were separated using Chiralpak® AD column.

Column dimensions: 21×250 mm, 10μ
Mobile phase: 50%:50%:0.1% Ethanol:Methanol:diethylamine
Flow rate (ml/min): 20 L/min

### Detection (nm): 220

### Post Purification Purity Check

Sample purity was checked with Chiralpak® AD.

Column dimensions: 4.6×250 mm, 5μ
Mobile phase: 60%:40%:0.1% CarbonDioxide:Methanol:dimethylethylamine Flow: 5 mL/min

### Detection: 220 nm

### Example 27(a)

### First Eluting Compound

### N²-[1-(5-Methoxypyrimidin-2-yl)ethyl]-N⁴-(1-methyl-1H-imidazol-4-yl)-7H-pyrrolo[2,3-d]pyrimidine-2,4-diamine, Enantiomer (A)

The first eluting compound had a retention time of 1.57 minutes, >98% ee.

¹H NMR (300 MHz, MeOD) δ ppm 8.49 (s, 2H), 8.04 (br. s., 1H), 7.26 (s, 1H), 6.97 (d, J=3.58 Hz, 1H), 6.61 (d, J=0.75 Hz, 1H), 5.30 (q, J=7.16 Hz, 1H), 3.95 (s, 3H), 3.89 (s, 3H), 1.64 (d, J=6.97 Hz, 3H).

LCMS: 365 [M+H]⁺.

### Example 27(b)

### Second Eluting Compound

### N²-[1-(5-Methoxypyrimidin-2-yl)ethyl]-N⁴-(1-methyl-1H-imidazol-4-yl)-7H-pyrrole[2,3-d]pyrimidine-2,4-diamine, Enantiomer (B)

The second eluting compound had a retention time of 3.51 minutes, >98% ee.

¹H NMR (300 MHz, MeOD) δ ppm 8.49 (s, 2H), 8.04 (br. s., 1H), 7.26 (s, 1H), 6.97 (d, J=3.58 Hz, 1H), 6.61 (d, J=0.75 Hz, 1H), 5.30 (q, J=7.16 Hz, 1H), 3.95 (s, 3H), 3.89 (s, 3H), 1.64 (d, J=6.97 Hz, 3H).

LCMS: 365 [M+H]⁺.

### Example 28

### N²-[(1S)-1-(5-Fluoropyrimidin-2-yl)ethyl]-6-methoxy-N⁴-(1-methyl-1H-imidazol-4-yl)quinazoline-2,4-diamine, Trifluoroacetic Acid Salt

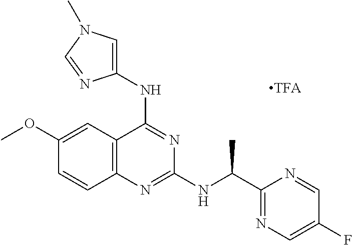

2-Chloro-6-methoxy-N-(1-methyl-1H-imidazol-4-yl)quinazolin-4-amine (Intermediate 49, 350 mg, 1.21 mmol) and (1S)-1-(5-fluoropyrimidin-2-yl)ethanamine hydrochloride (Intermediate 6, 428 mg, 2.42 mmol) were reacted using a procedure similar to the one described for the synthesis of Example 26. After purification by reversed phase HPLC (Gilson® chromatography, MeCN/0.1% TFA in water 5%→55%), the title product was provided as part of a mixture of enantiomers (390 mg), the title enantiomer being present in the mixture in an amount greater than or equal to the amount of the corresponding R enantiomer.

¹H NMR (300 MHz, MeOD) δ ppm 8.77 (s, 2H), 7.98 (d, 1H), 7.87 (s, 1H), 7.52 (d, 2H), 5.37-5.59 (m, 1H), 3.97 (s, 3H), 3.93 (s, 3H), 1.74 (d, 3H).

LCMS: 395 [M+H]⁺.

### Example 29

### N²-[1-(3,5-Difluoropyridin-2-yl)ethyl]-6-methoxy-N⁴-(1-methyl-1H-imidazol-4-yl)quinazoline-2,4-diamine, Trifluoroacetic Acid Salt

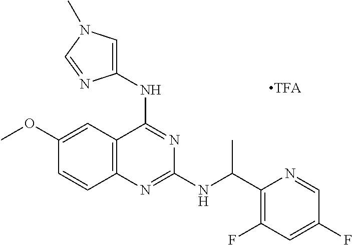

2-Chloro-6-methoxy-N-(1-methyl-1H-imidazol-4-yl)quinazolin-4-amine (Intermediate 49, 350 mg, 1.21 mmol) and 1-(3,5-difluoropyridin-2-yl)ethanamine hydrochloride (Intermediate 35, 558 mg, 2.42 mmol) were reacted using a procedure similar to the one described for the synthesis of Example 26, providing the title product (31 mg) as a racemic mixture, after purification by reversed phase HPLC (Gilson® chromatography, 0.1% TFA in water/MeCN 5%→55%).

¹H NMR (300 MHz, MeOD) δ ppm 8.35 (d, 1H), 7.50-7.72 (m, 4H), 7.30-7.47 (m, 2H), 5.57-5.76 (m, 1H), 3.93 (s, 3H), 3.84 (s, 3H), 1.61 (d, 3H).

LCMS: 411 [M+H]⁺.

### Example 30

### N²-[(1S)-1-(5-Fluoropyrimidin-2-yl)ethyl]-7-methoxy-N⁴-(1-methyl-1H-imidazol-4-yl)quinazoline-2,4-diamine, Trifluoroacetic Acid Salt

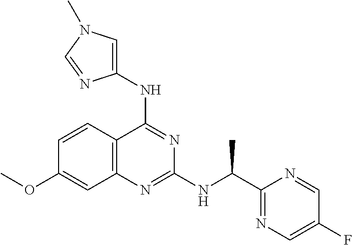

2-Chloro-7-methoxy-N-(1-methyl-1H-imidazol-4-yl)quinazolin-4-amine (Intermediate 52, 383 mg, 1.32 mmol) and (1S)-1-(5-fluoropyrimidin-2-yl)ethanamine hydrochloride (Intermediate 6, 468 mg, 2.64 mmol) were reacted using a procedure similar to the one described for the synthesis of Example 26. After purification utilizing reversed phase HPLC (Column: Waters XBridge C18 100×19 mm, particle size 5μ; Mobile phase: 0.1% NH₄OH in water/MeCN; Gradient: 10-60% Acetonitrile in 10 min (3 min wash)), the title product was provided as part of a mixture of enantiomers (160 mg), the title enantiomer being present in the mixture in an amount greater than or equal to the amount of the corresponding R enantiomer.

¹H NMR (300 MHz, MeOD) δ ppm 8.71 (s, 2H), 7.85-8.00 (m, 1H), 7.59 (br. s., 1H), 7.44 (d, 1H), 6.80 (dq, 2H), 5.48 (q, J=6.97 Hz, 1H), 3.89 (s, 3H), 3.82 (s, 3H), 1.64 (d, 3H).

LCMS: 394 [M+H]⁺.

### Column and Solvent Conditions

The R and S enantiomers were separated using Chiral SFC (Chiralpak® AD column).

Column dimensions: 21×250 mm, 5μ
Mobile phase: carbon dioxide:methanol:dimethylethylamine (65%:35%:0.4%)
Flow rate (ml/min): 60

### Detection (nm): 254

### Post Purification Purity Check

Sample purity was checked with Chiralpak® AD.

Column dimensions: 4.6×250 mm, 5μ
Mobile phase: 60%:44%:0.4% CarbonDioxide:Methanol:dimethylethylamine Flow: 2.5 mL/min

### Detection: 254 nm

### Example 30 (a)

### First Eluting Compound

### N²-[1-(5-Fluoropyrimidin-2-yl)ethyl]-7-methoxy-N⁴-(1-methyl-1H-imidazol-4-yl)quinazoline-2,4-diamine, Enantiomer (A)

The first eluting compound had a retention time of 4.46 minutes, >98% ee.

LCMS: 394 [M+H]⁺.

¹H NMR (300 MHz, MeOD) δ ppm 8.71 (s, 2H), 7.85-8.00 (m, 1H), 7.59 (br. s., 1H), 7.44 (d, J=1.32 Hz, 1H), 6.80 (dq, J=4.83, 2.47 Hz, 2H), 5.48 (q, J=6.97 Hz, 1H), 3.89 (s, 3H), 3.82 (s, 3H), 1.64 (d, J=6.97 Hz, 3H).

### Example 30(b)

### Second Eluting Compound

### N²-[1-(5-Fluoropyrimidin-2-yl)ethyl]-7-methoxy-N⁴-(1-methyl-1H-imidazol-4-yl)quinazoline-2,4-diamine, Enantiomer (B)

The second eluting compound had a retention time of 5.38 minutes, >98% ee

¹H NMR (300 MHz, MeOD) δ ppm 8.71 (s, 2H), 7.85-8.00 (m, 1H), 7.59 (br. s., 1H), 7.44 (d, J=1.32 Hz, 1H), 6.80 (dq, J=4.83, 2.47 Hz, 2H), 5.48 (q, J=6.97 Hz, 1H), 3.89 (s, 3H), 3.82 (s, 3H), 1.64 (d, J=6.97 Hz, 3H).

### Example 31

### N²-[1-(3,5-Difluoropyridin-2-yl)ethyl]-6-fluoro-N⁴-(1-methyl-1H-imidazol-4-yl)pyrido[2,3-d]pyrimidine-2,4-diamine, Trifluoroacetic Acid Salt

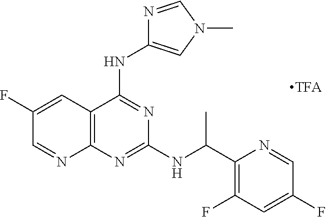

1-(3,5-Difluoropyridin-2-yl)ethanamine hydrochloride (Intermediate 35, 305 mg, 1.57 mmol) and 2-chloro-6-fluoro-N-(1-methyl-1H-imidazol-4-yl)pyrido[2,3-d]pyrimidin-4-amine (Intermediate 55, 364 mg, 1.31 mmol) were reacted using a procedure similar to the one described for the synthesis of Example 26, providing the title product (217 mg) as a racemic mixture, after purification by reversed phase HPLC (Gilson® chromatography, 0.1% TFA in water/MeCN 5%→50%).

¹H NMR (300 MHz, Chloroform-d) δ ppm 8.68 (d, J=2.83 Hz, 1H), 8.34 (m, 2H), 8.05 (br. s., 1H), 7.98 (br.s, 1H), 7.61 (br. s., 1H), 7.38 (m, 1H), 7.15-7.25 (m, 1H), 5.85 (br. s., 1H), 3.77 (br. s., 3H), 1.64 (d, J=3.96 Hz, 3H).

### Column and Solvent Conditions

The R and S enantiomers were separated using chiral HPLC.

Column dimensions: Chiralpak® IC 21×250 mm, 5μ
Mobile phase A: Hexane 70%
Mobile phase B: Hexane 1:1 Methanol:Ethanol 30%
Additive: 0.1% diethylamine
Flow rate: 20 (mL/min)

### Detection: 254 nm

### Post Purification Purity Check

Sample purity was checked with chiral HPLC using Chiralpak® IC

Column dimensions: 4.6×250 mm, 5μ
Mobile phase: Hexane/1:1 Methanol:Ethanol=1:1, Additive: 0.1% diethylamine
Flow: 1 mL/min

### Example 31(a)

### First Eluting Compound

### N²-[1-(3,5-Difluoropyridin-2-yl)ethyl]-6-fluoro-N⁴-(1-methyl-1H-imidazol-4-yl)pyrido[2,3-d]pyrimidine-2,4-diamine, Enantiomer (A)

The first eluting compound had a retention time of 14.31 min, >98% ee

¹H NMR (300 MHz, MeOD) δ ppm 1.48 (d, J=6.78 Hz, 3H) 3.55 (br. s., 3H) 5.52-5.78 (m, 1H) 7.09-7.42 (m, 1H) 7.47 (ddd, J=9.89, 8.57, 2.26 Hz, 2H) 8.10-8.40 (m, 2H) 8.50 (d, J=2.07 Hz, 1H).

LCMS: 400.9 M+H]⁺.

### Example 31(b)

### Second Eluting Compound

### N²-[1-(3,5-Difluoropyridin-2-yl)ethyl]-6-fluoro-N⁴-(1-methyl-1H-imidazol-4-yl)pyrido[2,3-d]pyrimidine-2,4-diamine, Enantiomer (B)

The second eluting compound had a retention time of 17.94 min, 98%.

¹H NMR (300 MHz, MeOD) δ ppm 1.48 (d, J=6.97 Hz, 3H) 3.54 (br. s., 3H) 5.46-5.84 (m, 1H) 7.10-7.38 (m, 1H) 7.38-7.67 (m, 2H) 8.17-8.39 (m, 2H) 8.49 (br. s., 1H).

LCMS: 400.9 M+H]⁺.

### Example 32

### N²-[(1S)-1-(5-Fluoropyrimidin-2-yl)ethyl]-N⁴-(1-methyl-1H-imidazol-4-yl)-7-(trifluoromethyl)pyrido[2,3-d]pyrimidine-2,4-diamine, Trifluoroacetic Acid Salt

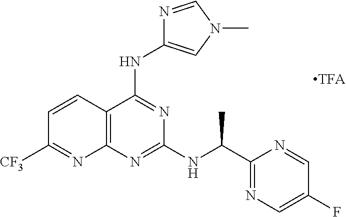

(1S)-1-(5-Fluoropyrimidin-2-yl)ethanamine hydrochloride (Intermediate 6, 454 mg, 2.56 mmol) and 2-chloro-N-(1-methyl-1H-imidazol-4-yl)-7-(trifluoromethyl)pyrido[2,3-d]pyrimidin-4-amine (Intermediate 59, 700 mg, 2.13 mmol) were reacted using a procedure similar to the one described for the synthesis of Example 26. After purification by reversed phase HPLC (Gilson® chromatography, 0.1% TFA in water/MeCN 5%→45%), the title product was provided as part of a mixture of enantiomers (101 mg), the title enantiomer being present in the mixture in an amount greater than or equal to the amount of the corresponding R enantiomer.

¹H NMR (300 MHz, MeOD) δ ppm 1.63 (d, J=6.97 Hz, 3H) 3.83 (s, 3H) 5.44 (q, J=7.03 Hz, 1H) 7.58 (s, 1H) 7.70 (d, J=8.29 Hz, 1H) 7.95 (s, 1H) 8.68 (s, 2H) 8.80 (d, J=8.10 Hz, 1H).

LCMS: 434.2 [M+H]

### Example 33

### N²-[1-(3,5-Difluoropyridin-2-yl)ethyl]-N⁴-(1-methyl-1H-imidazol-4-yl)-7-(trifluoromethyl)pyrido[2,3-d]pyrimidine-2,4-diamine, Trifluoroacetic Acid Salt

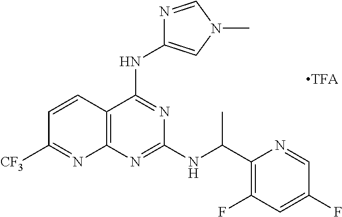

1-(3,5-Difluoropyridin-2-yl)ethanamine hydrochloride (Intermediate 35, 209 mg, 0.91 mmol) and 2-chloro-N-(1-methyl-1H-imidazol-4-yl)-7-(trifluoromethyl)pyrido[2,3-d]pyrimidin-4-amine (Intermediate 59, 250 mg, 0.76 mmol) were reacted using a procedure similar to the one described for the synthesis of Example 26, providing the title product (101 mg) as a racemic mixture, after purification by reversed phase HPLC (Gilson® chromatography, MeCN/0.1% TFA in water 5%→45%).

¹H NMR (300 MHz, MeOD) δ ppm 1.58 (d, J=6.78 Hz, 3H) 3.81 (s, 3H) 5.63 (q, J=7.03 Hz, 1H) 7.42-7.64 (m, 2H) 7.70 (d, J=8.29 Hz, 1H) 7.91 (br. s., 1H) 8.30 (d, J=2.26 Hz, 1H) 8.80 (d, J=8.10 Hz, 1H).

LCMS: 451.0[M+H]⁺.

### Example 34

### 2-{[(1S)-1-(5-Fluoropyrimidin-2-yl)ethyl]amino}-4-[(1-methyl-1H-imidazol-4-yl)amino]pyrido[2,3-d]pyrimidin-7-ol hydrochloride

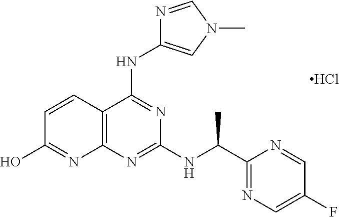

7-Chloro-N²-[(1S)-1-(5-fluoropyrimidin-2-yl)ethyl]-N⁴-(1-methyl-1H-imidazol-4-yl)pyrido[2,3-d]pyrimidine-2,4-diamine, Trifluoroacetic Acid Salt

(Example 42, 200 mg, 0.50 mmol) was added to a 5:1 (v/v) mixture of acetic acid (5 mL, 83.26 mmol) and water (1 mL, 55.51 mmol). The yellow solution was stirred for 24 hours at 100° C. Evaporation of the volatiles under reduced pressure gave a residue, which was purified by reversed phase HPLC (Gilson® chromatography, MeCN/0.1% formic acid in water 5%→20%) to give a yellow solid. The formate salt was converted into the HCl salt (the title product) by dissolving the former in 5 ml MeOH and subsequent addition of 1.25M HCl in MeOH (1 mL). Evaporation of the volatiles under reduced pressure gave the title product as part of a mixture of enantiomers in the form of a white solid (35.0 mg), the title enantiomer being present in the mixture in an amount greater than or equal to the amount of the corresponding R enantiomer.

¹H NMR (300 MHz, MeOD) δ ppm 1.56 (d, J=6.97 Hz, 3H) 3.94 (br. s., 3H) 5.29 (q, J=6.72 Hz, 1H) 6.36-6.72 (m, 1H) 7.57 (s, 1H) 8.28 (br. s., 1H) 8.54-8.71 (m, 2H) 8.83 (br. s., 1H).

LCMS: 382.1 [M+H]⁺.

### Example 35

### 2-{[1-(3,5-Difluoropyridin-2-yl)ethyl]amino}-4-[(1-methyl-1H-imidazol-4-yl)amino]pyrido[2,3-d]pyrimidin-7-ol hydrochloride

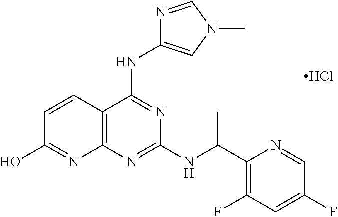

7-Chloro-N²-[1-(3,5-difluoropyridin-2-yl)ethyl]-N⁴-(1-methyl-1H-imidazol-4-yl)pyrido[2,3-d]pyrimidine-2,4-diamine, trifluoroacetic acid salt (Example 44, 55 mg, 0.13 mmol) was reacted using a procedure similar to the one described for the synthesis of Example 34. Purification by reversed phase HPLC (Gilson® chromatography, MeCN/0.1% formic acid in water 5%→25%) gave the corresponding formate salt (101 mg). Treatment of the formate salt with HCl solution (4N HCl in dioxane) provided the title product (12.50 mg) as a racemic mixture.

¹H NMR (300 MHz, MeOD) δ ppm 1.51 (d, J=6.22 Hz, 3H) 3.96 (br. s., 3H) 5.31-5.70 (m, 1H) 6.38-6.75 (m, 1H) 7.40-7.69 (m, 2H) 8.29 (d, J=1.51 Hz, 1H) 8.39 (d, J=8.85 Hz, 1H) 8.91 (br. s., 1H).

LCMS: 399.1 [M+H]⁺.

### Example 36

### N⁷-Cyclopropyl-N²-[(1S)-1-(5-fluoropyrimidin-2-yl)ethyl]-N⁴-(1-methyl-1H-imidazol-4-yl)pyrido[2,3-d]pyrimidine-2,4,7-triamine, Trifluoroacetic Acid Salt

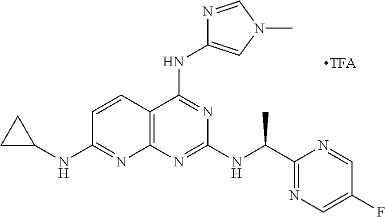

Cyclopropanamine (0.102 mL, 1.45 mmol) was added to a mixture of 7-chloro-N²-[(1S)-1-(5-fluoropyrimidin-2-yl)ethyl]-N⁴-(1-methyl-1H-imidazol-4-yl)pyrido[2,3-d]pyrimidine-2,4-diamine, Trifluoroacetic Acid Salt

(Example 42, 145 mg, 0.36 mmol) and DIPEA (0.253 mL, 1.45 mmol) in BuOH (3 mL). The mixture was heated at 140° C. overnight. Evaporation of the volatiles under reduced pressure gave a residue. Purification by reversed phase HPLC (Gilson® chromatography, MeCN/0.1% TFA in water 5%→35%) gave the title product as part of a mixture of enantiomers, the title enantiomer being present in the mixture in an amount greater than or equal to the amount of the corresponding R enantiomer.

¹H NMR (300 MHz, MeOD) δ ppm 0.35-0.61 (m, 2H) 0.78 (d, 2H) 1.19-1.38 (m, 1H) 1.58 (d, J=6.97 Hz, 3H) 3.72 (s, 3H) 5.35 (q, 1H) 6.51 (d, J=12.81 Hz, 1H) 7.29 (br. s., 1H) 7.40 (s, 1H) 8.13 (d, 1H) 8.66 (s, 2H).

LCMS: 421.3 [M+H]⁺.

### Example 37

### N²-[(1S)-1-(5-Fluoropyrimidin-2-yl)ethyl]-N⁴-(1-methyl-1H-imidazol-4-yl)-7-morpholin-4-ylpyrido[2,3-d]pyrimidine-2,4-diamine, Trifluoroacetic Acid Salt

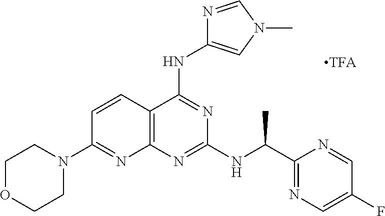

7-Chloro-N²-[(1S)-1-(5-fluoropyrimidin-2-yl)ethyl]-N⁴-(1-methyl-1H-imidazol-4-yl)pyrido[2,3-d]pyrimidine-2,4-diamine, Trifluoroacetic Acid Salt

(Example 42,120 mg, 0.3 mmol) and morpholine (0.105 mL, 1.20 mmol) were reacted using a procedure similar to the one described for the synthesis of Example 36. After purification by reversed phase HPLC (Gilson® chromatography, MeCN/0.1% TFA in water 5%→35%), the title product was provided as part of a mixture of enantiomers (65.0 mg), the title enantiomer being present in the mixture in an amount greater than or equal to the amount of the corresponding R enantiomer.

¹H NMR (300 MHz, MeOD) δ ppm 1.58 (d, J=6.59 Hz, 3H) 3.63-3.77 (m, 8H) 3.81 (s, 3H) 5.34 (q, J=7.10 Hz, 1H) 6.88 (br. s., 1H) 7.42 (br. s., 1H) 7.93 (br. s., 1H) 8.24 (br. s., 1H) 8.48-8.83 (m, 2H).

LCMS: 451.0 [M+H]

### Example 38

### 6-Fluoro-N²-[(1S)-1-(5-fluoropyrimidin-2-yl)ethyl]-N⁴-(1-methyl-1H-imidazol-4-yl)pyrido[2,3-d]pyrimidine-2,4-diamine, Trifluoroacetic Acid Salt

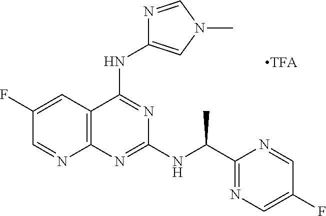

2-Chloro-6-fluoro-N-(1-methyl-1H-imidazol-4-yl)pyrido[2,3-d]pyrimidin-4-amine (Intermediate 55, 90 mg, 0.32 mmol) and (1S)-1-(5-fluoropyrimidin-2-yl)ethanamine hydrochloride (Intermediate 6) were reacted using a procedure similar to the one described for the synthesis of Example 26. After purification by reversed phase HPLC (Gilson® chromatography, MeCN/0.1% TFA in water 5%→30%), the title product was provided as part of a mixture of enantiomers (139 mg), the title enantiomer being present in the mixture in an amount greater than or equal to the amount of the corresponding R enantiomer.

### Column and Solvent Conditions

The R and S enantiomers were separated using chiral HPLC.

Column dimensions: Chiralpak® IC 20×250 mm, 5μ,
Mobile phase A: Hexane 70%
Mobile phase B: Hexane 1:1 Methanol:Ethanol 30%
Additive: 0.1% diethylamine
Flow rate (ml/min): 20 mL/min

### Detection (nm): 220

### Post Purification Purity Check

Sample purity was checked by chiral HPLC with Chiralpak® IC column

Column dimensions: 4.6×250 mm, 5μ
Mobile phase: 1:1 Methanol:Ethanol, Additive: 0.1% diethylamine
Flow: 1 mL/min

### Example 38(a)

### First Eluting Compound

### 6-Fluoro-N²-[1-(5-fluoropyrimidin-2-yl)ethyl]-N⁴-(1-methyl-1H-imidazol-4-yl)pyrido[2,3-d]pyrimidine-2,4-diamine, Enantiomer (A)

The first eluting compound had a retention time of 16.02 min, >98% ee.

¹H NMR (300 MHz, DMSO-d6) δ ppm 1.50 (d, J=6.97 Hz, 3H) 3.65 (s, 3H) 5.17-5.50 (m, 1H) 7.41 (s, 1H) 7.59 (d, J=8.10 Hz, 1H) 8.55 (br. s., 1H) 8.70 (d, J=9.04 Hz, 1H) 8.79 (s, 2H) 10.26 (br. s., 1H).

LCMS: 394.1 [M+H]⁺.

### Example 38(b)

### Second Eluting Compound

### 6-Fluoro-N²-[1-(5-fluoropyrimidin-2-yl)ethyl]-N⁴-(1-methyl-1H-imidazol-4-yl)pyrido[2,3-d]pyrimidine-2,4-diamine, Enantiomer (B)

The second eluting compound had a retention time of 19.88 min, 97.4% ee.

¹H NMR (300 MHz, DMSO-d6) δ ppm 1.50 (d, J=6.97 Hz, 3H) 3.65 (s, 3H) 5.23-5.43 (m, 1H) 7.41 (s, 1H) 7.58 (d, J=7.91 Hz, 1H) 8.55 (br. s., 1H) 8.70 (d, J=7.35 Hz, 1H) 8.78 (s, 2H) 10.25 (s, 1H).

LCMS: 394.9 [M+H]⁺.

### Example 39

### N²,N⁷-bis[(1S)-1-(5-fluoropyrimidin-2-yl)ethyl]-N⁴-(1-methyl-1H-imidazol-4-yl)pyrido[2,3-d]pyrimidine-2,4,7-triamine

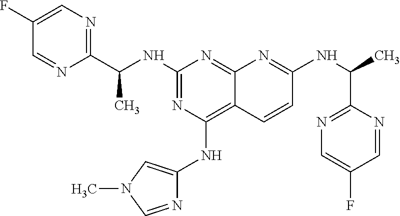

Upon the reaction conditions reported for the preparation of 7-chloro-N²-[(1S)-1-(5-fluoropyrimidin-2-yl)ethyl]-N⁴-(1-methyl-1H-imidazol-4-yl)pyrido[2,3-d]pyrimidine-2,4-diamine, Trifluoroacetic Acid Salt (Example 42), the title product was formed as a by-product as part of a mixture of enantiomers (105 mg).

¹H NMR (300 MHz, MeOD) δ ppm 1.53 (d, J=6.97 Hz, 6H) 3.82 (s, 3H) 5.15-5.50 (m, 2H) 6.66 (d, 1H) 7.40 (br. s., 1H) 7.89-8.18 (m, 2H) 8.53-8.72 (m, 4H).

LCMS: 505.1 [M+H]⁺.

### Example 40

### N²-[1-(3,5-Difluoropyridin-2-yl)ethyl]-N⁴-(1-methyl-1H-imidazol-4-yl)-7-morpholin-4-ylpyrido[2,3-d]pyrimidine-2,4-diamine, Trifluoroacetic Acid Salt

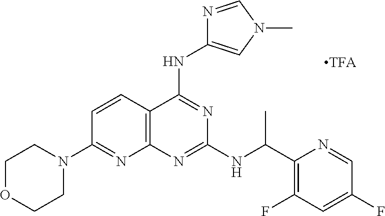

7-Chloro-N²-[1-(3,5-difluoropyridin-2-yl)ethyl]-N⁴-(1-methyl-1H-imidazol-4-yl)pyrido[2,3-d]pyrimidine-2,4-diamine, trifluoroacetic acid salt (Example 44, 237 mg, 0.57 mmol) and morpholine (0.198 mL, 2.27 mmol) were reacted using a procedure similar to the one described for the synthesis of Example 36. After purification by reversed phase HPLC (Gilson® chromatography, MeCN/0.1% TFA in water 5%→35%) the title product was provided as a racemic mixture (201 mg).

### Column and Solvent Conditions

The R and S enantiomers were separated using chiral HPLC.

Column dimensions: Chiralpak® AD 20×250 mm, 10μ
Mobile phase A: Hexane 70%
Mobile phase B: Hexane 1:1 Methanol:Ethanol 30%
Additive: 0.1% diethylamine
Flow rate: 20 mL/min

### Detection (nm): 254

### Post Purification Purity Check

Sample purity was checked by chiral HPLC

### Column: Chiralpak® AD

Column dimensions: 4.6×250 mm, 5μ
Mobile phase: 1:1 Methanol:Ethanol, Additive: 0.1% diethylamine
Flow: 1 mL/min

### Example 40(a)

### First Eluting Compound

### N²-[1-(3,5-Difluoropyridin-2-yl)ethyl]-N⁴-(1-methyl-1H-imidazol-4-yl)-7-morpholin-4-ylpyrido[2,3-d]pyrimidine-2,4-diamine, Enantiomer (A)

The first eluting compound had a retention time of 11.42 min, >95.5% e.e.

¹H NMR (300 MHz, MeOD) δ ppm 1.46 (d, J=6.97 Hz, 3H) 3.65 (d, J=6.78 Hz, 11H) 5.45-5.80 (m, 1H) 6.59 (d, 1H) 7.31 (s, 1H) 7.45 (ddd, 2H) 8.05 (d, 1H) 8.23 (d, 1H).

LCMS: 468.2 [M+H]⁺.

### Example 40(b)

### Second Eluting Compound

### N²-[1-(3,5-Difluoropyridin-2-yl)ethyl]-N⁴-(1-methyl-1H-imidazol-4-yl)-7-morpholin-4-ylpyrido[2,3-d]pyrimidine-2,4-diamine, Enantiomer (B)

The second eluting compound had a retention time of 15 min, 98% e.e.

¹H NMR (300 MHz, MeOD) δ ppm 1.45 (d, J=6.97 Hz, 3H) 3.53-3.76 (m, 11H) 5.40-5.80 (m, 1H) 6.57 (d, J=9.04 Hz, 1H) 7.30 (s, 1H) 7.44 (ddd, J=9.94, 8.62, 2.35 Hz, 2H) 8.04 (d, J=9.04 Hz, 1H) 8.23 (d, J=2.26 Hz, 1H).

LCMS: 468.2 [M+H]⁺.

### Example 41

### N²-[(1S)-1-(5-Fluoropyrimidin-2-yl)ethyl]-N⁴-(1-methyl-1H-imidazol-4-yl)pyrido[3,4-d]pyrimidine-2,4-diamine, Trifluoroacetic Acid Salt

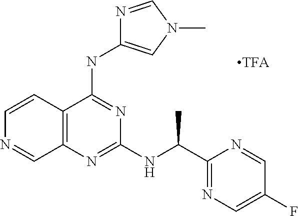

2-Chloro-N-(1-methyl-1H-imidazol-4-yl)pyrido[3,4-d]pyrimidin-4-amine (Intermediate 67, 404 mg, 1.55 mmol) and (1S)-1-(5-Fluoropyrimidin-2-yl)ethanamine hydrochloride (Intermediate 6), were reacted using a procedure similar to the one described for the synthesis of Example 26. After purification by reversed phase HPLC (Gilson® chromatography, MeCN/0.1% TFA in water 5%→40%), the title product was provided as part of a mixture of enantiomers (209 mg), the title enantiomer being present in the mixture in an amount greater than or equal to the amount of the corresponding R enantiomer.

### Column and Solvent Conditions

The R and S enantiomers were separated using chiral HPLC.

Column dimensions: Chiralpak® AD 20×250 mm, 10μ
Mobile phase: 1:1 Methanol:Ethanol, Additive: 0.1% diethylamine
Flow rate (ml/min): 20 mL/min

### Detection (nm): 220

### Post Purification Purity Check

Sample purity was checked by chiral HPLC

### Column: Chiralpak® AD

Column dimensions: 4.6×250 mm, 10μ
Mobile phase: 1:1 Methanol:Ethanol, Additive: 0.1% diethylamine
Flow: 1 mL/min

### Example 41(a)

### First Eluting Compound

### N²-[1-(5-Fluoropyrimidin-2-yl)ethyl]-N⁴-(1-methyl-1H-imidazol-4-yl)pyrido[3,4-d]pyrimidine-2,4-diamine, Enantiomer (A)

The first eluting compound had a retention time of 7.48 min, >98% ee.

¹H NMR (300 MHz, MeOD) δ ppm 1.50 (d, J=6.97 Hz, 3H) 3.58 (br. s., 3H) 5.61 (q, J=6.78 Hz, 1H) 7.06-7.41 (m, 1H) 7.41-7.62 (m, 2H) 7.82 (d, 1H) 8.07 (dd, 1H) 8.27 (s, 1H) 8.55 (d, 1H).

LCMS: 367.0 [M+H]⁺.

### Example 41(b)

### Second Eluting Compound

### N²-[1-(5-Fluoropyrimidin-2-yl)ethyl]-N⁴-(1-methyl-1H-imidazol-4-yl)pyrido[3,4-d]pyrimidine-2,4-diamine, Enantiomer (B)

The second eluting compound had a retention time of 11.36 min, >98% ee.

¹H NMR (300 MHz, MeOD) δ ppm 1.51 (d, J=6.97 Hz, 3H) 3.60 (br. s., 3H) 5.61 (q, J=6.91 Hz, 1H) 7.27 (br. s., 1H) 7.43-7.63 (m, 2H) 7.83 (d, 1H) 8.08 (d, 1H) 8.27 (d, 1H) 8.57 (s, 1H).

LCMS: 367.0 [M+H]⁺.

### Example 42

### 7-Chloro-N²-[(1S)-1-(5-fluoropyrimidin-2-yl)ethyl]-N⁴-(1-methyl-1H-imidazol-4-yl)pyrido[2,3-d]pyrimidine-2,4-diamine, Trifluoroacetic Acid Salt

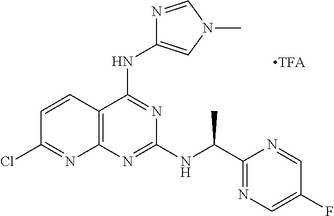

2,7-Dichloro-N-(1-methyl-1H-imidazol-4-yl)pyrido[2,3-d]pyrimidin-4-amine (Intermediate 63, 385 mg, 1.30 mmol) and (1S)-1-(5-fluoropyrimidin-2-yl)ethanamine hydrochloride (Intermediate 6, 323 mg, 1.30 mmol) were reacted using a procedure similar to the one described for the synthesis of Example 26. Purification by reversed phase HPLC (Gilson® chromatography, MeCN/0.1% TFA in water 5%→35%) provided the title product as part of a mixture of enantiomers (181 mg), the title enantiomer being present in the mixture in an amount greater than or equal to the amount of the corresponding R enantiomer.

¹H NMR (300 MHz, MeOD) δ ppm 1.62 (d, J=6.97 Hz, 3H) 3.82 (s, 3H) 5.40 (q, J=6.91 Hz, 1H) 7.39 (d, 1H) 7.45-7.59 (m, 1H) 7.90 (s, 1H) 8.57 (d, 1H) 8.67 (s, 2H).

LCMS: 399.9 [M+H]⁺.

### Example 43

### N²-[1-(3,5-Difluoropyridin-2-yl)ethyl]-N⁴-(1-methyl-1H-imidazol-4-yl)pyrido[3,4-d]pyrimidine-2,4-diamine

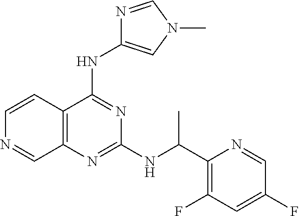

2-Chloro-N-(1-methyl-1H-imidazol-4-yl)pyrido[3,4-d]pyrimidin-4-amine (Intermediate 67, 102 mg, 0.39 mmol) and 1-(3,5-difluoropyridin-2-yl)ethanamine hydrochloride (Intermediate 35, 74.3 mg, 0.47 mmol) were reacted using a procedure similar to the one described for the synthesis of Example 26, providing the title product (105 mg) as a racemic mixture, after purification by reversed phase HPLC (Gilson® chromatography, MeCN/0.1% ammonium acetate in water 5%→55%).

### Column and Solvent Conditions

The R and S enantiomers were separated using chiral HPLC.

Column dimensions: Chiralpak® AD 20×250 mm, 10μ
Mobile phase: 1:1 Methanol:Ethanol, Additive: 0.1% diethylamine
Flow rate (ml/min): 20 mL/min

### Detection (nm): 220

### Post Purification Purity Check

Sample purity was checked by chiral HPLC

### Column: Chiralpak® AD

Column dimensions: 4.6×250 mm, 10μ
Mobile phase: 1:1 Methanol:Ethanol, Additive: 0.1% diethylamine
Flow: 1 mL/min

### Example 43(a)

### First Eluting Compound

### N²-[1-(3,5-Difluoropyridin-2-yl)ethyl]-N⁴-(1-methyl-1H-imidazol-4-yl)pyrido[3,4-d]pyrimidine-2,4-diamine, Enantiomer (A)

The first eluting compound had a retention time of 6.4 min, >98% ee.

¹H NMR (300 MHz, MeOD) δ ppm 1.50 (d, J=6.97 Hz, 3H) 3.58 (br. s., 3H) 5.61 (q, J=6.78 Hz, 1H) 7.06-7.41 (m, 1H) 7.41-7.62 (m, 2H) 7.82 (d, J=4.33 Hz, 1H) 8.07 (dd, J=5.46, 3.20 Hz, 1H) 8.27 (s, 1H) 8.55 (d, J=1.70 Hz, 1H).

LCMS: 383.1 [M+H]⁺.

### Example 43(b)

### Second Eluting Compound

### N²-[1-(3,5-Difluoropyridin-2-yl)ethyl]-N⁴-(1-methyl-1H-imidazol-4-yl)pyrido[3,4-d]pyrimidine-2,4-diamine, Enantiomer (B)

The second eluting compound had a retention time of 9.73 min, >98% ee.

¹H NMR (300 MHz, MeOD) δ ppm 1.51 (d, J=6.97 Hz, 3H) 3.60 (br. s., 3H) 5.61 (q, J=6.91 Hz, 1H) 7.27 (br. s., 1H) 7.43-7.63 (m, 2H) 7.83 (d, J=5.46 Hz, 1H) 8.08 (d, J=5.65 Hz, 1H) 8.27 (d, J=2.26 Hz, 1H) 8.57 (s, 1H).

LCMS: 383.1 [M+H]⁺.

### Example 44

### 7-Chloro-N²-[1-(3,5-difluoropyridin-2-yl)ethyl]-N⁴-(1-methyl-1H-imidazol-4-yl)pyrido[2,3-d]pyrimidine-2,4-diamine, Trifluoroacetic Acid Salt

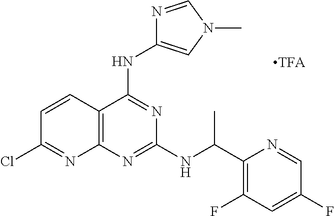

2,7-Dichloro-N-(1-methyl-1H-imidazol-4-yl)pyrido[2,3-d]pyrimidin-4-amine (Intermediate 63, 400 mg, 1.36 mmol) and 1-(3,5-difluoropyridin-2-yl)ethanamine hydrochloride (Intermediate 35) were reacted using a procedure similar to the one described for the synthesis of Example 26, providing the title product (209 mg) as a racemic mixture, after purification by reversed phase HPLC (Gilson® chromatography, MeCN/0.1% TFA in water 5%→50%).

¹H NMR (300 MHz, MeOD) δ ppm 1.56 (d, J=6.97 Hz, 3H) 3.79 (s, 3H) 5.61 (q, J=6.47 Hz, 1H) 7.25-7.65 (m, 3H) 7.75 (br. s., 1H) 8.29 (d, 1H) 8.58 (d, 1H).

LCMS: 417.0 [M+H]⁺.

### Example 45

### N²-[(1S)-1-(5-Fluoropyrimidin-2-yl)ethyl]-N⁴-(1-methyl-1H-imidazol-4-yl)quinazoline-2,4-diamine, Trifluoroacetic Acid Salt

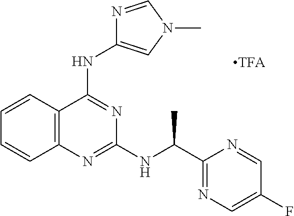

2-Chloro-N-(1-methyl-1H-imidazol-4-yl)quinazolin-4-amine (Intermediate 68, 460 mg, 1.77 mmol), and (1S)-1-(5-fluoropyrimidin-2-yl)ethanamine hydrochloride (Intermediate 6, 378 mg, 2.13 mmol) in n-BuOH (4 mL). The mixture was heated at 150° C. under microwave irradiation for 6 hour. The mixture was cooled at room temperature and the volatiles were evaporated in vacuo to give a residue. Purification by reversed phase HPLC (Gilson® chromatography, MeCN/0.1% TFA in water 5%→50%) provided the title product as part of a mixture of enantiomers, the title enantiomer being present in the mixture in an amount greater than or equal to the amount of the corresponding R enantiomer.

¹H NMR (300 MHz, MeOD) δ ppm 8.66 (s, 2H), 8.21 (d, J=8.10 Hz, 2H), 8.00 (br. s., 1H), 7.79 (td, J=7.82, 1.32 Hz, 1H), 7.47 (m, 2H), 5.38 (q, J=6.40 Hz, 1H), 3.84 (s, 3H), 1.61 (d, J=6.78 Hz, 3H).

LCMS: 383 [M+H]⁺.

### Column and Solvent Conditions

The R and S enantiomers were separated using Chiral SFC.

Column dimensions: Chiralpak® AD 21×250 mm, 5μ
Mobile phase A: Carbon Dioxide 75%
Mobile phase B: 1:1 Methanol:Ethanol, Additive: 0.4% diethylamine 25%
Flow rate (ml/min): 60 mL/min

### Detection (nm): 254

### Temperature (° C.): 40

Outlet Pressure (bar): 100

### Post Purification Purity Check

Sample purity was checked by SFC

### Column: Chiralpak® AD

Column dimensions: 4.6×100 mm, 5μ
Mobile phase A: Carbon Dioxide 80%
Mobile phase B: 1:1 Methanol:Ethanol, Additive: 0.4% diethylamine 20%
Flow rate (ml/min): 5 mL/min

### Detection (nm): 220

### Temperature (° C.): 35

Outlet Pressure (bar): 120

### Example 45(a)

### First Eluting Compound

### N²-[1-(5-Fluoropyrimidin-2-yl)ethyl]-N⁴-(1-methyl-1H-imidazol-4-yl)quinazoline-2,4-diamine, Enantiomer (A)

The first eluting compound had a retention time of 2.69 min, >98% ee.

¹H NMR (300 MHz, MeOD) δ ppm 8.60 (s, 2H), 7.92 (d, J=8.29 Hz, 1H), 7.48 (m, 2H), 7.34 (s, 1H), 7.26 (d, J=8.48 Hz, 1H), 7.07 (m, 1H), 5.38 (q, J=6.97 Hz, 1H), 3.72 (s, 3H), 1.53 (d, J=6.97 Hz, 3H).

LCMS: 383 [M+H]⁺.

### Example 45(b)

### Second Eluting Compound

### N²-[1-(5-Fluoropyrimidin-2-yl)ethyl]-N⁴-(1-methyl-1H-imidazol-4-yl)quinazoline-2,4-diamine, Enantiomer (B)

The second eluting compound had a retention time of 3.86 min, >98% ee.

¹H NMR (300 MHz, MeOD) δ ppm 8.59 (s, 2H), 7.90 (dd, 1H), 7.47 (m, 2H), 7.32 (d, J=1.13 Hz, 1H), 7.25 (d, J=7.91 Hz, 1H), 7.06 (m, 1H), 5.37 (q, J=6.97 Hz, 1H), 3.70 (s, 3H), 1.53 (d, J=6.97 Hz, 3H).

LCMS: 383 [M+H]⁺.

### Example 46

### N⁶-[(1S)-1-(5-Fluoropyrimidin-2-yl)ethyl]-N⁴-(1-methyl-1H-imidazol-4-yl)-1H-pyrazolo[3,4-d]pyrimidine-4,6-diamine

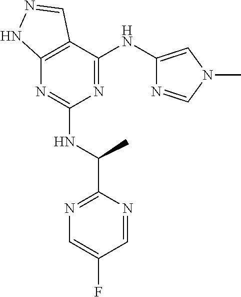

6-Chloro-N-(1-methyl-1H-imidazol-4-yl)-1-(tetrahydro-2H-pyran-2-yl)-1H-pyrazolo[3,4-d]pyrimidin-4-amine (Intermediate 69, 158 mg, 0.47 mmol) and (1S)-1-(5-fluoropyrimidin-2-yl)ethanamine hydrochloride (Intermediate 6, 84 mg, 0.47 mmol) were dissolved in butan-1-ol (2.5 mL), followed by the addition of triethylamine (0.165 mL, 1.18 mmol). The reaction mixture was heated under microwave irradiation at 160° C. for 6 hours. LCMS analysis indicated that the protecting group was cleaved under the employed conditions. The volatiles were evaporated under reduced pressure and the residue was purified to give the title product as part of a mixture of enantiomers (14.2 mg), the title enantiomer being present in the mixture in an amount greater than or equal to the amount of the corresponding R enantiomer.

¹H NMR (300 MHz, MeOD) δ ppm 8.70 (s, 2H), 7.87 (br.s, 1H), 7.55 (br. s, 1H), 7.56 (br. s, 1H), 5.40 (q., 1H), 3.81 (s, 3H), 1.62 (d, 3H).

LCMS: 355 [M+H]⁺.

What is claimed is:

1. A compound of Formula (I): 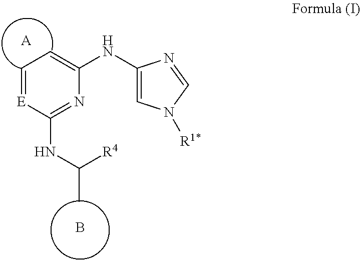
or a pharmaceutically acceptable salt thereof, wherein:
Ring A is selected from fused 5- or 6-membered heterocycle and fused 5- or 6-membered carbocycle, wherein said fused 5- or 6-membered heterocycle and fused 5- or 6-membered carbocycle are optionally substituted on carbon with one or more R², and wherein if said 5- or 6-membered fused heterocycle contains an —NH— moiety, that —NH— moiety is optionally substituted with R²*;
Ring B is 5- or 6-membered heteroaryl, wherein said 5- or 6-membered heteroaryl is optionally substituted on carbon with one or more R⁵, and wherein if said 5- or 6-membered heteroaryl contains an —NH— moiety, that —NH— moiety is optionally substituted with R⁵*;
E is selected from N and C—R³,
R¹* is selected from H, —CN, C₁₋₆alkyl, carbocyclyl, heterocyclyl, —OR^{1a}, —N(R^{1a})₂, —C(O)H, —C(O)R^{1b}, —C(O)₂R^{1a}, —C(O)N(R^{1a})₂, —S(O)R^{1b}, —S(O)₂R^{1b}, —S(O)₂N(R^{1a})₂, —C(R^{1a})═N(R^{1a}), and —C(R^{1a})═N(OR^{1a}), wherein said C₁₋₆alkyl, carbocyclyl, and heterocyclyl are optionally substituted on carbon with one or more R¹⁰, and wherein if said heterocyclyl contains an —NH— moiety, that —NH— moiety is optionally substituted with R¹⁰*;
R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein any —NH— moiety of said heterocyclyl is optionally substituted with R¹⁰*;
R² in each occurrence is independently selected from halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —N(R^{2a})OR^{2a}, —ON(R^{2a})₂, —C(O)H, —C(O)R^{2b}, —C(O)₂R^{2a}, —C(O)N(R^{2a})₂, —C(O)N(R^{2a})(OR^{2a})—OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —C(R^{2a})═N(R^{2a}), and —C(R^{2a})═N(OR^{2a}), wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein any —NH— moiety of said heterocyclyl is optionally substituted with R²⁰*;
R²* in each occurrence is independently selected from C₁₋₆alkyl, carbocyclyl, heterocyclyl, —C(O)H, —C(O)R^{2b}, —C(O)₂R^{2a}, —C(O)N(R^{2a})₂, —S(O)R^{2b}, —S(O)₂R^{2b}, —S(O)₂N(R^{2a})₂, —C(R^{2a})═N(R^{2a}), and —C(R^{2a})═N(OR^{2a}), wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein if said heterocyclyl contains an —NH— moiety, that —NH— moiety is optionally substituted with R²⁰*;
R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —N(R^{3a})(OR^{3a}), —O—N(R^{3a})₂, —C(O)H, —C(O)R^{3b}, —C(O)₂R^{3a}, —C(O)N(R^{3a})₂, —C(O)N(R^{3a})(OR^{3a}), —OC(O)N(R^{3a})₂, —N(R^{3a})C(O)₂R³, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —C(R^{3a})═N(R^{3a}), and —C(R^{3a})═N(OR^{3a}), wherein said C₁₋₆alkyl, C₂₋₆alkenyl, C₂₋₆alkynyl, carbocyclyl, and heterocyclyl are optionally substituted on carbon with one or more R³⁰, and wherein if said heterocyclyl contains an —NH— moiety, that —NH— moiety is optionally substituted with R³⁰*;
R^{3a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein if said heterocyclyl contains an —NH— moiety, that —NH— moiety is optionally substituted with R³⁰*;
R^{3b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein if said heterocyclyl contains an —NH— moiety, that —NH— moiety is optionally substituted with R³⁰*;
R⁴ is selected from H, halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —N(R^{4a})(OR^{4a}), —O—N(R^{4a})₂, —C(O)H, —C(O)R^{4b}, —C(O)₂R^{4a}, —C(O)N(R^{4a})₂, —C(O)N(R^{4a})(OR^{4a})—OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S(O)₂R^{4b}, —C(R^{4a})═N(R^{4a}), and —C(R^{4a})═N(OR^{4a}), wherein said C₁₋₆alkyl, C₂₋₆alkenyl, C₂₋₆alkynyl, carbocyclyl, and heterocyclyl are optionally substituted on carbon with one or more R⁴⁰, and wherein if said heterocyclyl contains an —NH— moiety, that —NH— moiety is optionally substituted with R⁴⁰*;
R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein if said heterocyclyl contains an —NH— moiety, that —NH— moiety is optionally substituted with R⁴⁰*;
R^{4b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein if said heterocyclyl contains an —NH— moiety, that —NH— moiety is optionally substituted with R⁴⁰*;
R⁵ in each occurrence is independently selected from H, halo, —CN, C₁₋₆alkyl, C₂₋₆alkenyl, C₂₋₆alkynyl, carbocyclyl, heterocyclyl, —OR^{5a}, —SR^{5a}, —N(R^{5a})₂, —N(R^{5a})C(O)R^{5b}, —N(R^{5a})N(R^{5a})₂, —NO₂, —N(R^{5a})(OR^{5a}), —O—N(R^{5a})₂, —C(O)H, —C(O)R^{5b}, —C(O)₂R^{5a}, —C(O)N(R^{5a})₂, —C(O)N(R^{5a})(OR^{5a})—OC(O)N(R^{5a})₂, —N(R^{5a})C(O)₂R^{5a}, —N(R^{5a})C(O)N(R^{5a})₂, —OC(O)R^{5b}, —S(O)R^{5b}, —S(O)₂R^{5b}, —S(O)₂N(R^{5a})₂, —N(R^{5a})S(O)₂R^{5b}, —C(R^{5a})═N(R^{5a}), and —C(R^{5a})═N(OR^{5a}), wherein said C₁₋₆alkyl, C₂₋₆alkenyl, C₂₋₆alkynyl, carbocyclyl, and heterocyclyl in each occurrence are optionally and independently substituted on carbon with one or more R⁵⁰, and wherein if said heterocyclyl contains an —NH— moiety, that —NH— moiety is optionally substituted with R⁵⁰*;
R⁵* in each occurrence is independently selected from H, —CN, C₁₋₆alkyl, carbocyclyl, heterocyclyl, —OR^{5a}, —N(R^{5a})₂, —C(O)H, —C(O)R^{5b}, —C(O)₂R^{5a}, —C(O)N(R^{5a})₂, —S(O)R^{5b}, —S(O)₂R^{5b}, —S(O)₂N(R^{5a})₂, —C(R^{5a})═N(R^{5a}), and —C(R^{5a})═N(OR^{5a}), wherein said C₁₋₆alkyl, carbocyclyl, and heterocyclyl in each occurrence are optionally and independently substituted on carbon with one or more R⁵⁰, and wherein if said heterocyclyl contains an —NH— moiety, that —NH— moiety is optionally substituted with R⁵⁰*;
R^{5a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁵⁰, and wherein if said heterocyclyl contains an —NH— moiety, that —NH— moiety is optionally substituted with R⁵⁰*;
R^{5b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁵⁰, and wherein if said heterocyclyl contains an —NH— moiety, that —NH— moiety is optionally substituted with R⁵⁰*;
R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})C(O)R^{10b}, —N(R^{10a})N(R^{10a})₂, —NO₂, —N(R^{10a})(OR^{10a}), —O—N(R^{10a})₂, —C(O)H, —C(O)R^{10b}, —C(O)₂R^{10a}, —C(O)N(R^{10a})₂, —C(O)N(R^{10a})(OR^{10a}), —OC(O)N(R^{10a})₂, —N(R^{10a})C(O)₂R^{10a}, —N(R^{10a})C(O)N(R^{10a})₂, —OC(O)R^{10b}, —S(O)R^{10b}, —S(O)₂R^{10b}, —S(O)₂N(R^{10a})₂, —N(R^{10a})S(O)₂R^{10b}, —C(R^{10a})═N(R^{10a}), and —C(R^{10a})═N(OR^{10a});
R¹⁰* in each occurrence is independently selected from C₁₋₆alkyl, carbocyclyl, heterocyclyl, —C(O)H, —C(O)R^{10b}, —C(O)₂R^{10a}, —C(O)N(R^{10a})₂, —S(O)R^{10b}, —S(O)₂R^{10b}, —S(O)₂N(R^{10a})₂, —C(R^{10a})═N(R^{10a}), and —C(R^{10a})═N(OR^{10a});
R^{10a} in each occurrence is independently selected from H, C₁₋₆alkyl, carbocyclyl, and heterocyclyl;
R^{10b} in each occurrence is independently selected from C₁₋₆alkyl, C₂₋₆alkenyl, C₂₋₆alkynyl, carbocyclyl, and heterocyclyl;
R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})C(O)R^{20b}, —N(R^{20a})N(R^{20a})₂, NO₂, —N(R^{20a})—OR^{20a}, —O—N(R^{20a})₂, —C(O)H, —C(O)R^{20b}, —C(O)₂R^{20a}, —C(O)N(R^{20a})₂, —C(O)N(R^{20a})(OR^{20a}), —OC(O)N(R^{20a})₂, —N(R^{20a})C(O)₂R^{20a}, —N(R^{20a})C(O)N(R^{20a})₂, —OC(O)R^{20b}, —S(O)R^{20b}, —S(O)₂R^{20b}, —S(O)₂N(R^{20a})₂, —N(R^{20a})S(O)₂R^{20b}, —C(R^{20a})N(R^{20a}), and —C(R^{20a})═N(OR^{20a}), wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R²⁰* in each occurrence is independently selected from —CN, C₁₋₆alkyl, carbocyclyl, heterocyclyl, —OR^{20a}, —N(R^{20a})₂, —C(O)H, —C(O)R^{20b}, —C(O)₂R^{20a}, —C(O)N(R^{20a})₂, —S(O)R^{20b}, —S(O)₂R^{20b}, —S(O)₂N(R^{20a})₂, —C(R^{20a})═N(R^{20a}), and —C(R^{20a})═N(OR^{20a}), wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —N(R^{30a})(OR^{30a}), —O—N(R^{30a})₂, —C(O)H, —C(O)R^{30b}, —C(O)₂R^{30a}, —C(O)N(R^{30a})₂, —C(O)N(R^{30a})(OR^{30a}), —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —C(R^{30a})═N(R^{30a}), and —C(R^{30a})═N(OR^{30a});
R³⁰* in each occurrence is independently selected from —CN, C₁₋₆alkyl, carbocyclyl, heterocyclyl, —OR^{30a}, —N(R^{30a})₂, —C(O)H, —C(O)R^{30b}, —C(O)₂R^{30a}, —C(O)N(R^{30a})₂, —S(O)R^{30b}, —S(O)₂R^{30b}, —S(O)₂N(R^{30a})₂, —C(R^{30a})═N(R^{30a}), and —C(R^{30a})═N(OR^{30a});
R^{30a} in each occurrence is independently selected from H, C₁₋₆alkyl, carbocyclyl, and heterocyclyl;
R^{30b} in each occurrence is independently selected from C₁₋₆alkyl, C₂₋₆alkenyl, C₂₋₆alkynyl, carbocyclyl, and heterocyclyl;
R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})C(O)R^{40b}, —N(R^{40a})N(R^{40a})₂—NO₂, —N(R^{40a})(OR^{40a}), —O—N(R^{40a})₂, —C(O)H, —C(O)R^{40b}, —C(O)₂R^{40a}, —C(O)N(R^{40a})₂, —C(O)N(R^{40a})(OR^{40a}), —OC(O)N(R^{40a})₂, —N(R^{40a})C(O)₂R^{40a}, —N(R^{40a})C(O)N(R^{40a})₂, —OC(O)R^{40b}, —S(O)R^{40b}, —S(O)₂R^{40b}, —S(O)₂N(R^{40a})₂, —N(R^{40a})S(O)₂R^{40b}, —C(R^{40a})═N(R^{40a}), and —C(R^{40a})═N(OR^{40a});
R⁴⁰* in each occurrence is independently selected from —CN, C₁₋₆alkyl, carbocyclyl, heterocyclyl, —OR^{40a}, —N(R^{40a})₂, —C(O)H, —C(O)R^{40b}, —C(O)₂R^{40a}, —C(O)N(R^{40a})₂, —S(O)R^{40b}, —S(O)₂R^{40b}, —S(O)₂N(R^{40a})₂, —C(R^{40a})═N(R^{40a}), and —C(R^{40a})═N(OR^{40a});
R^{40a} in each occurrence is independently selected from H, C₁₋₆alkyl, carbocyclyl, and heterocyclyl;
R^{40b} in each occurrence is independently selected from C₁₋₆alkyl, C₂₋₆alkenyl, C₂₋₆alkynyl, carbocyclyl, and heterocyclyl;
R⁵⁰ in each occurrence is independently selected from halo, —CN, C₁₋₆alkyl, C₂₋₆alkenyl, C₂₋₆alkynyl, carbocyclyl, heterocyclyl, —OR^{50a}, —SR^{50a}, —N(R^{50a})₂, —N(R^{50a})C(O)R^{50b}, —N(R^{50a})N(R^{50a})₂, —NO₂, —N(R^{50a})(OR^{50a}), —O—N(R^{50a})₂, —C(O)H, —C(O)R^{50b}, —C(O)₂R^{50a}, —C(O)N(R^{50a})₂, —C(O)N(R^{50a})(OR^{50a}), —OC(O)N(R^{50a})₂, —N(R^{50a})C(O)₂R^{50a}, —N(R^{50a})C(O)N(R^{50a})₂, —OC(O)R^{50b}, —S(O)R^{50b}, —S(O)₂R^{50b}, —S(O)₂N(R^{50a})₂, —N(R^{50a})S(O)₂R^{50b}, —C(R^{50a})═N(R^{50a}), and —C(R^{50a})═N(OR^{50a});
R⁵⁰* in each occurrence is independently selected from —CN, C₁₋₆alkyl, carbocyclyl, heterocyclyl, —OR^{50a}, —N(R^{50a})₂, —C(O)H, —C(O)R^{50b}, —C(O)₂R^{50a}, —C(O)N(R^{50a})₂, —S(O)R^{50b}, —S(O)₂R^{50b}, —S(O)₂N(R^{50a})₂, —C(R^{50a})═N(R^{50a}), and —C(R^{50a})═N(OR^{50a});
R^{50a} in each occurrence is independently selected from H, C₁₋₆alkyl, carbocyclyl, and heterocyclyl;
R^{50b} in each occurrence is independently selected from C₁₋₆alkyl, C₂₋₆alkenyl, C₂₋₆alkynyl, carbocyclyl, and heterocyclyl;
R^{b} in each occurrence is independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂, —N(R^{m})C(O)Rⁿ, —N(R^{m})N(R^{m})₂, —NO₂, —N(R^{m})—OR^{m}, —O—N(R^{m})₂, —C(O)H, —C(O)Rⁿ, —C(O)₂R^{m}, —C(O)N(R^{m})₂, —C(O)N(R^{m})(OR^{m}), —OC(O)N(R^{m})₂, —N(R^{m})C(O)₂R^{m}, —N(R^{m})C(O)N(R^{m})₂, —OC(O)Rⁿ, —S(O)Rⁿ, —S(O)₂Rⁿ, —S(O)₂N(R^{m})₂, —N(R^{m})S(O)₂Rⁿ, —C(R^{m})═N(R^{m}), and —C(R^{m})═N(OR^{m});
R^{b}* in each occurrence is independently selected from —CN, C₁₋₆alkyl, carbocyclyl, heterocyclyl, —OR^{m}, —N(R^{m})₂, —C(O)H, —C(O)Rⁿ, —C(O)₂R^{m}, —C(O)N(R^{m})₂, —S(O)Rⁿ, —S(O)₂Rⁿ, —S(O)₂N(R^{m})₂, —C(R^{m})═N(R^{m}), and —C(R^{m})═N(OR^{m});
R^{m} in each occurrence is independently selected from H, C₁₋₆alkyl, carbocyclyl, and heterocyclyl; and
Rⁿ in each occurrence is independently selected from C₁₋₆alkyl, C₂₋₆alkenyl, C₂₋₆alkynyl, carbocyclyl, and heterocyclyl.

2. A compound of Formula (I), or a pharmaceutically acceptable salt thereof, as claimed in claim 1, wherein E is N.

3. A compound of Formula (I), or a pharmaceutically acceptable salt thereof, as claimed in claim 1, wherein:
Ring A is selected from fused 5- or 6-membered heterocycle and fused 5- or 6-membered carbocycle, wherein said fused 5- or 6-membered heterocycle and fused 5- or 6-membered carbocycle are optionally substituted on carbon with one or more R², and wherein any —NH— moiety of said fused 5- or 6-membered heterocycle is optionally substituted with R²*;
R² in each occurrence is independently selected from halo, C₁₋₆alkyl, 5- or 6-membered heterocyclyl, —OR^{2a}, and —N(R^{2a})₂, wherein said C₁₋₆alkyl is optionally substituted with one or more R²⁰;
R²* in each occurrence is independently selected from C₁₋₆alkyl and 3- to 5-membered carbocyclyl, wherein said C₁₋₆alkyl is optionally substituted with one or more R²⁰;
R^{2a} in each occurrence is independently selected from H, C₁₋₆alkyl, and 3- to 5-membered carbocyclyl; and
R²⁰ in each occurrence is independently selected from halo and —OH.

4. A compound of Formula (I), or a pharmaceutically acceptable salt thereof, as claimed in claim 1, wherein:
Ring B is 6-membered heteroaryl, wherein said 6-membered heteroaryl is optionally substituted with one or more R⁵; and
R⁵ is halo.

5. A compound of Formula (I), or a pharmaceutically acceptable salt thereof, as claimed in claim 1, wherein R¹* is C₁₋₆alkyl.

6. A compound of Formula (I), or a pharmaceutically acceptable salt thereof, as claimed in claim 1, wherein:
R⁴ is C₁₋₆alkyl, wherein said C₁₋₆alkyl is optionally substituted with one or more R⁴⁰;
R⁴⁰ is —OR^{40a}; and
R^{40a} is C₁₋₆alkyl.

7. A compound of Formula (Ia): 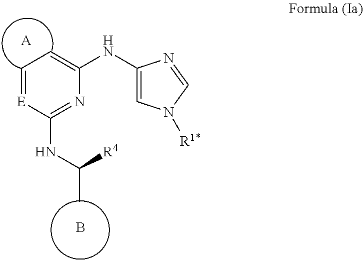
or a pharmaceutically acceptable salt thereof, wherein:
Ring A, together with the pyrimidine to which it is fused, forms a member selected from 7-cyclopropyl-7H-pyrrolo[2,3-d]pyrimidine, 5,7-dihydro-6H-pyrrolo[3,4-d]pyrimidine, 6,7-dihydro-5H-cyclopenta[d]pyrimidine, 1-ethyl-1H-pyrazolo[3,4-d]pyrimidine, 7-(2-fluoroethyl)-7H-pyrrolo[2,3-d]pyrimidine, 7-methoxyquinazoline, 9-methyl-9H-purine, 1-methyl-1H-pyrazolo[3,4-d]pyrimidine, 6-methyl-7H-pyrrolo[2,3-d]pyrimidine, 7-methyl-7H-pyrrolo[2,3-d]pyrimidine, 7-methylthieno[3,2-d]pyrimidine, pteridine, 1H-pyrazolo[3,4-d]pyrimidine, 2-(1H-pyrazolo[3,4-d]pyrimidin-1-yl)ethanol, pyrido[2,3-d]pyrimidine, pyrido[3,4-d]pyrimidine, 5H-pyrrolo[3,2-d]pyrimidine, 7H-pyrrolo[2,3-d]pyrimidine, 5,6,7,8-tetrahydropyrido[4,3-d]pyrimidine, thieno[2,3-d]pyrimidine, and 6-(trifluoromethyl)-7H-pyrrolo[2,3-d]pyrimidine;
Ring B is selected from 3,5-difluoropyridin-2-yl and 5-fluoropyrimidin-2-yl;
E is N;
R¹* is methyl; and
R⁴ is selected from methyl and methoxymethyl.

8. A compound selected from:
N²-[(1S)-1-(5-fluoropyrimidin-2-yl)ethyl]-N⁴-(1-methyl-1H-imidazol-4-yl)thieno[2,3-d]pyrimidine-2,4-diamine;
N²-[(1S)-1-(5-fluoropyrimidin-2-yl)ethyl]-7-methyl-N⁴-(1-methyl-1H-imidazol-4-yl)thieno[3,2-d]pyrimidine-2,4-diamine;
N²-[(1S)-1-(5-fluoropyrimidin-2-yl)ethyl]-N⁴-(1-methyl-1H-imidazol-4-yl)-7H-pyrrolo[2,3-d]pyrimidine-2,4-diamine;
N²-[(1S)-1-(5-fluoropyrimidin-2-yl)ethyl]-N⁴-(1-methyl-1H-imidazol-4-yl)-5H-pyrrolo[3,2-d]pyrimidine-2,4-diamine;
N²-[(1S)-1-(5-fluoropyrimidin-2-yl)ethyl]-N⁴-(1-methyl-1H-imidazol-4-yl)-6,7-dihydro-5H-cyclopenta[c]pyrimidine-2,4-diamine;
N⁶-[(1S)-1-(5-fluoropyrimidin-2-yl)ethyl]-1-methyl-N⁴-(1-methyl-1H-imidazol-4-yl)-1H-pyrazolo[3,4-d]pyrimidine-4,6-diamine;
N²-[(1S)-1-(3,5-difluoropyridin-2-yl)ethyl]-N⁴-(1-methyl-1H-imidazol-4-yl)pyrido[2,3-d]pyrimidine-2,4-diamine;
N⁶-[(1R)-1-(3,5-difluoropyridin-2-yl)-2-methoxyethyl]-1-methyl-N⁴-(1-methyl-1H-imidazol-4-yl)-1H-pyrazolo[3,4-d]pyrimidine-4,6-diamine;
N⁶-[(1S)-1-(3,5-difluoropyridin-2-yl)ethyl]-1-methyl-N⁴-(1-methyl-1H-imidazol-4-yl)-1H-pyrazolo[3,4-d]pyrimidine-4,6-diamine;
2-(6-{[(1S)-1-(5-fluoropyrimidin-2-yl)ethyl]amino}-4-[(1-methyl-1H-imidazol-4-yl)amino]-1H-pyrazolo[3,4-d]pyrimidin-1-yl)ethanol;
N²-[(1S)-1-(5-fluoropyrimidin-2-yl)ethyl]-N⁴-(1-methyl-1H-imidazol-4-yl)pyrido[2,3-d]pyrimidine-2,4-diamine;
N²-[(1S)-1-(5-fluoropyrimidin-2-yl)ethyl]-7-methyl-N⁴-(1-methyl-1H-imidazol-4-yl)-7H-pyrrolo[2,3-d]pyrimidine-2,4-diamine;
N²-[(1S)-1-(5-fluoropyrimidin-2-yl)ethyl]-7-methoxy-N⁴-(1-methyl-1H-imidazol-4-yl)quinazoline-2,4-diamine;
2-{[(1S)-1-(3,5-difluoropyridin-2-yl)ethyl]amino}-4-[(1-methyl-1H-imidazol-4-yl)amino]pyrido[2,3-d]pyrimidin-7-ol;
N⁷-cyclopropyl-N²-[(1S)-1-(5-fluoropyrimidin-2-yl)ethyl]-N⁴-(1-methyl-1H-imidazol-4-yl)pyrido[2,3-d]pyrimidine-2,4,7-triamine;
N²,N⁷-bis[(1S)-1-(5-fluoropyrimidin-2-yl)ethyl]-N⁴-(1-methyl-1H-imidazol-4-yl)pyrido[2,3-d]pyrimidine-2,4,7-triamine;
N²-[(1S)-1-(3,5-difluoropyridin-2-yl)ethyl]-N⁴-(1-methyl-1H-imidazol-4-yl)-7-morpholin-4-ylpyrido[2,3-d]pyrimidine-2,4-diamine;
N²-[(1S)-1-(5-fluoropyrimidin-2-yl)ethyl]-N⁴-(1-methyl-1H-imidazol-4-yl)pyrido[3,4-d]pyrimidine-2,4-diamine;
7-chloro-N²-[(1S)-1-(5-fluoropyrimidin-2-yl)ethyl]-N⁴-(1-methyl-1H-imidazol-4-yl)pyrido[2,3-d]pyrimidine-2,4-diamine;
N²-[(1S)-1-(3,5-difluoropyridin-2-yl)ethyl]-N⁴-(1-methyl-1H-imidazol-4-yl)pyrido[3,4-d]pyrimidine-2,4-diamine;
7-chloro-N²-[(1S)-1-(3,5-difluoropyridin-2-yl)ethyl]-N⁴-(1-methyl-1H-imidazol-4-yl)pyrido[2,3-d]pyrimidine-2,4-diamine; or
N²-[(1S)-1-(5-fluoropyrimidin-2-yl)ethyl]-N⁴-(1-methyl-1H-imidazol-4-yl)quinazoline-2,4-diamine,
or a pharmaceutically acceptable salt thereof.

9. A compound of Formula (I), or a pharmaceutically acceptable salt thereof, as claimed in claim 1, for use as a medicament.

10. The use of a compound of Formula (I), or a pharmaceutically acceptable salt thereof, as claimed in claim 1, in the manufacture of a medicament for the treatment of cancer.

11. A method for treating cancer in a warm-blooded animal such as man, said method comprising administering to said animal an effective amount of a compound of Formula (I), or a pharmaceutically acceptable salt thereof, as claimed in claim 1.

12. A compound of Formula (I), or a pharmaceutically acceptable salt thereof, as claimed in claim 1, for use in the treatment of cancer in a warm-blooded animal such as man.

13. A pharmaceutical composition comprising a compound of Formula (I), or a pharmaceutically acceptable salt thereof, as claimed in claim 1, and at least one pharmaceutically acceptable carrier, diluent, or excipient.

14. A process for preparing a compound of Formula (I), or a pharmaceutically acceptable salt thereof, as claimed in claim 1:
reacting a compound of Formula (A): 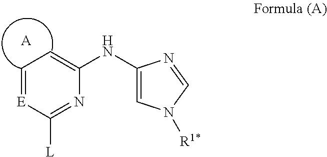
with a compound of Formula (B): 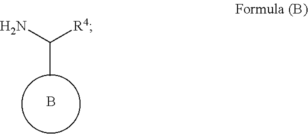
and thereafter if necessary:
i) converting a compound of Formula (I) into another compound of Formula (I);
ii) removing any protecting groups; and/or
iii) forming a pharmaceutically acceptable salt,
wherein L is a leaving group.
